SEQUENCE LISTING (5 sequences)

SEQ ID NO: 1 - DNA, 366 bp, Artificial Sequence
  Description of Artificial Sequence Synthetic polynucleotide
atgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctac

SEQ ID NO: 2 - DNA, 19 bp, Artificial Sequence
  Description of Artificial Sequence Synthetic oligonucleotide
gcacgagcatcagccgtgc

SEQ ID NO: 3 - DNA, 65 bp, Artificial Sequence
  Description of Artificial Sequence Synthetic oligonucleotide
atcctacggtacttattgccaagaaagcacgagcatcagccgtgcctccaggtcgaatcttcaaa

SEQ ID NO: 4 - DNA, 366 bp, Artificial Sequence
  Description of Artificial Sequence Synthetic polynucleotide
atgttggcggtaaagatggcttctaactttacgcagttcgttctcgtagacaatggcgggactggggacgtaacagtcgccccatctaattttgctaatggaatagcggagtggataagcagtaatagccgaagccaggcctataaggtgacatgctccgtgcgacaatccagtgctcaaaatcgaaaatacaccattaaagtagaagtccctaagggcgcctggcgatcctaccttaacatggagctcactattccaatctttgctaccaattctgactgcgagctgatagtaaaagcaatgcagggtcttttgaaggacggcaacccgattccgtccgctattgctgcaaatagcgggatttac

SEQ ID NO: 5 - protein, 6 aa, Artificial Sequence
  Description of Artificial Sequence Synthetic 6xHis tag
HHHHHH

(19) United States
(12) Patent Application Publication
(10) Pub. No.: US 2023/0125614 A1
(43) Pub. Date: Apr. 27, 2023

(54) IN VIVO mRNA DISPLAY: LARGE-SCALE PROTEOMICS BY NEXT GENERATION SEQUENCING

(71) Applicant: The Trustees of Columbia University in the City of New York, New York, NY (US)

(72) Inventors: Saeed TAVAZOIE, New York, NY (US); Panagiotis OIKONOMOU, New York, NY (US)

(21) Appl. No.: 17/905,583

(22) PCT Filed: Mar. 5, 2021
(86) PCT No.: PCT/US2021/021249

§ 371 Date: Sep. 2, 2022

Related U.S. Application Data
(60) Provisional application No. 62/985,538, filed on Mar. 5, 2020.

(51) Int. Cl. C12N 15/10

(52) U.S. Cl. CPC C12N 15/1062

(57) ABSTRACT

Described herein is a method for producing in vivo mRNA displayed proteins by linking of in vivo expressed proteins to in vivo expressed RNA sequences that enable specific downstream identification of the individual proteins through nucleic acid-based sequencing. RNA-protein linkage is enabled by fusing an RNA-binding domain (e.g., MS2 coat protein) to a protein of interest that is expressed in a cell or compartment in which is also expressed the RNA sequence harboring both the recognition element for the RNA-binding domain (e.g., MS2 stem-loop) and an identifying sequence that uniquely maps to the protein of interest. The identifying sequence can be the coding sequence corresponding to the protein of interest or any RNA barcode that by design uniquely corresponds to the protein of interest. As such, libraries of such in vivo mRNA displayed proteins can be assayed in parallel for a variety of protein behaviors and functions.

This application claims the benefit of and priority to U.S. Provisional Patent Application No. 62/985,538, filed on Mar. 5, 2020, the content of which is hereby incorporated by reference in its entirety.

### GOVERNMENT SUPPORT

This invention was made with government support under Grant No. NHGRI (NIH) R01-HG009065-05 awarded by the National Institute of Health and the National Human Genome Research Institute. The Government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### BACKGROUND

Molecular interactions are the fundamental units of information processing in living cells. Almost every attempt to study a biological process eventually culminates in the characterization of physical interactions between the molecular components that make up the system. The most fundamental of such molecular interactions occur between: (1) proteins and proteins: beyond their physical architectural role, these interactions form the backbone of signaling and decision-making circuits that lie at the core of cellular behavior; (2) proteins and DNA: these interactions are the key to both DNA replication and transcription, two of the most fundamentally important processes in living cells; (3) proteins and RNA: these interactions regulate critical fates of messenger-RNA molecules, including stability, splicing, localization, and translation.

### SUMMARY OF THE INVENTION

In certain aspects, the invention provides a nucleic acid comprising a mRNA display cassette, the mRNA display cassette comprising a cloning site for insertion of a nucleotide sequence encoding a protein of interest operably linked to (i) a nucleotide sequence encoding a MS2 bacteriophage coat protein (MCP) and (ii) to a nucleotide sequence encoding an RNA stem-loop, wherein the MCP binds to the RNA stem-loop with high-affinity.

In certain aspects, the invention provides a nucleic acid comprising (i) first cassette comprising a cloning site for insertion of a nucleotide sequence encoding a protein of interest operably linked to a nucleotide sequence encoding a MS2 bacteriophage coat protein (MCP) and (ii) a second cassette comprising a nucleotide sequence encoding a unique molecular identifier (UMI) sequence operably linked to a nucleotide sequence encoding an RNA stem-loop, wherein the MCP binds to the RNA stem-loop with high-affinity.

In some embodiments, the nucleotide sequence encoding the MCP is located 5′ to the cloning site for insertion of the nucleotide sequence encoding the protein of interest. In some embodiments, the nucleotide sequence encoding the RNA stem-loop is located 3′ to the cloning site for insertion of the nucleotide sequence encoding the protein of interest. In some embodiments, the nucleotide sequence encoding the RNA stem-loop is located in a 3′ UTR.

In some embodiments, the mRNA display cassette is configured so that upon insertion of a nucleotide sequence encoding a protein of interest, the nucleotide sequence encoding the protein of interest and the MCP are operably linked so that they encode a fusion protein of the protein of interest and the MCP. In some embodiments, the fusion protein comprises the MCP fused to the N-terminus of the protein of interest.

In some embodiments, the mRNA display cassette further comprises a nucleotide sequence encoding a purification tag operably linked to the cloning site for insertion of the nucleic acid sequence encoding the protein of interest. In some embodiments, the mRNA display cassette is configured so that upon insertion of a nucleotide sequence encoding a protein of interest, the nucleotide sequence encoding the protein of interest and the purification tag are operably linked so that they encode a fusion protein of the protein of interest and the purification tag. In some embodiments, the fusion protein comprises the purification tag fused to the C-terminus of the protein of interest. In some embodiments, the nucleic acid further comprises a promoter operably linked to the mRNA display cassette. In some embodiments, the promoter is an inducible promoter.

In some embodiments, the mRNA display cassette further comprises a nucleotide sequence encoding a protein of interest. In some embodiments, the nucleotide sequence encoding the protein of interest comprises one or more deletions, insertions, or substitutions as compared to its wild-type sequence. In some embodiments, the protein of interest comprises a peptide. In some embodiments, the peptide comprises an artificial or in silico designed peptide. In some embodiments, the protein of interest encoded by the nucleotide sequence comprises one or more deletions, insertions, or substitutions as compared to its wild-type sequence.

In certain aspects, the invention provides a vector comprising any one of the nucleic acids disclosed herein.

In certain aspects, the invention provides a host cell comprising any vector disclosed herein.

In certain aspects, the invention provides a population of nucleic acids, each nucleic acid of the population comprising a mRNA display cassette, the mRNA display cassette comprising a nucleotide sequence encoding a protein of interest operably linked to (i) a nucleotide sequence encoding a MS2 bacteriophage coat protein (MCP) and (ii) to a nucleotide sequence encoding an RNA stem-loop, wherein the MCP binds to the RNA stem-loop with high-affinity.

In certain aspects, the invention provides a population of nucleic acids, each nucleic acid of the population comprising (i) a first cassette comprising a cloning site for insertion of a nucleotide sequence encoding a protein of interest operably linked to a nucleotide sequence encoding a MS2 bacteriophage coat protein (MCP) and (ii) a second cassette comprising a nucleotide sequence encoding a unique molecular identifier (UMI) sequence operably linked to a nucleotide sequence encoding an RNA stem-loop, wherein the MCP binds to the RNA stem-loop with high-affinity.

In some embodiments, for each nucleic acid of the population the nucleotide sequence encoding the MCP is located 5′ to the nucleotide sequence encoding the protein of interest. In some embodiments, for each nucleic acid of the population the nucleotide sequence encoding the RNA stem-loop is located 3′ to the nucleotide sequence encoding the protein of interest. In some embodiments, for each nucleic acid of the population the nucleotide sequence encoding the RNA stem-loop is located in a 3′ UTR. In some embodiments, for each nucleic acid of the population the nucleotide sequence encoding the protein of interest and the MCP are operably linked so that they encode a fusion protein of the protein of interest and the MCP. In some embodiments, for each nucleic acid of the population the fusion protein comprises the MCP fused to the N-terminus of the protein of interest.

In some embodiments, for each nucleic acid of the population the mRNA display cassette further comprises a nucleotide sequence encoding a purification tag operably linked to the nucleic acid sequence encoding the protein of interest. In some embodiments, for each nucleic acid of the population the nucleotide sequence encoding the protein of interest and the purification tag are operably linked so that they encode a fusion protein of the protein of interest and the purification tag. In some embodiments, for each nucleic acid of the population the fusion protein comprises the purification tag fused to the C-terminus of the protein of interest. In some embodiments, each nucleic acid of the population further comprises a promoter operably linked to the mRNA display cassette. In some embodiments, the promoter is an inducible promoter.

In some embodiments, for each nucleic acid of the population the mRNA display cassette further comprises a nucleotide sequence encoding a protein of interest. In some embodiments, at least one of the nucleic acids of the population comprises a nucleotide sequence encoding a protein of interest, wherein the nucleotide sequence comprises one or more deletions, insertions, or substitutions as compared to its wild-type sequence. In some embodiments, at least one of the nucleic acids of the population comprises a nucleotide sequence encoding a protein of interest, wherein the protein of interest encoded by the nucleotide sequence comprises one or more deletions, insertions, or substitutions as compared to its wild-type sequence.

In some embodiments, each nucleic acid of the population comprises a nucleotide sequence encoding a different protein of interest. In some embodiments, the protein of interest comprises a peptide. In some embodiments, the peptide comprises an artificial or in silico designed peptide. In some embodiments, the nucleic acids of the population comprise nucleotide sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest.

In some embodiments, the nucleic acids of the population comprises nucleotide sequences encoding different proteins of interest that are representative of a proteome of interest. In some embodiments, the proteome of interest is the proteome of Saccharomyces cerevisiae. In some embodiments, each nucleic acid of the population of nucleic acids is in a vector.

In certain aspects, the invention provides a population of host cells, wherein each host cell comprises a vector from the population of vectors disclosed herein.

In certain aspects, the invention provides a method of producing a population of cells comprising an in vivo mRNA display library, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; b) allowing the expression of the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest.

In certain aspects, the invention provides a method of producing a population of cells comprising an in vivo mRNA display library, the method comprising: a) providing a population of cells, wherein each cell comprises one or more nucleic acid sequences encoding: a RNA-binding protein; a protein of interest; a unique molecular identifier (UMI); and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the unique molecular identifier comprises the cognate RNA sequence; b) allowing the expression of the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the unique molecular identifier.

In some embodiments, the RNA-binding protein is an RNA-binding capsid protein and optionally, wherein the cognate RNA-binding sequence is an RNA stem-loop sequence. In some embodiments, the RNA-binding capsid protein is MS2 bacteriophage coat protein (MCP). In some embodiments, the nucleic acid sequence encoding the RNA-binding protein is located 5′ to the nucleic acid sequence encoding the protein of interest. In some embodiments, the nucleic acid sequence encoding the cognate RNA sequence is located 3′ to the nucleic acid sequence encoding the protein of interest. In some embodiments, the nucleic acid sequence encoding the cognate RNA sequence is located in a 3′ UTR of the mRNA sequence encoding the protein of interest. In some embodiments, the fusion protein comprises the RNA-binding protein fused to the N-terminus of the protein of interest.

In some embodiments, the one or more nucleic acid sequences further encodes a purification tag wherein the nucleic acid sequence encoding the protein of interest and the purification tag are operably linked so that they encode a fusion protein of the protein of interest and the purification tag. In some embodiments, the fusion protein comprises the purification tag fused to the C-terminus of the protein of interest. In some embodiments, each cell in the population of cells comprises a nucleic acid sequence encoding the same protein of interest. In some embodiments, each cell in the population of cells comprises a nucleic acid sequence encoding a different protein of interest. In some embodiments, the population of cells comprise nucleic acid sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest. In some embodiments, the population of cells comprise nucleic acids sequences encoding different proteins of interest that are representative of a proteome of interest.

In some embodiments, the nucleic acid sequence encoding the protein of interest comprises one or more deletions, insertions, or mutations as compared to its wild-type sequence. In some embodiments, the protein of interest encoded by the nucleic acid sequence comprises one or more deletions, insertions, or mutations as compared to its wild-type sequence.

In certain aspects, the invention provides a method of performing high throughput proteomics, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the protein of interest; d) lysing the population of cells; e) performing a biochemical assay on a portion of the lysate of step d) to generate an enriched lysate; f) detecting the amount of mRNA encoding each protein of interest and comprising the RNA stem-loop that is present in the lysate of step d); g) detecting the amount of mRNA encoding each protein of interest and comprising the RNA stem-loop that is present in the enriched lysate of step e); and h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).

In certain aspects, the invention provides a method of performing high throughput proteomics, the method comprising: a) providing a population of cells, wherein each cell comprises one or more nucleic acid sequences encoding: a RNA-binding protein; a protein of interest; a unique molecular identifier (UMI); and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the UMI comprises the cognate RNA sequence;

wherein the population of cells comprise nucleic acid sequences encoding a plurality of different proteins of interest; b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the UMI; d) lysing the population of cells; e) performing a biochemical assay on a portion of the lysate of step d) to generate an enriched lysate; f) detecting the amount of mRNA encoding each UMI and comprising the RNA stem-loop that is present in the lysate of step d); g) detecting the amount of mRNA encoding each UMI and comprising the RNA stem-loop that is present in the enriched lysate of step e); and h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).

In some embodiments, the RNA-binding domain is an RNA-binding capsid protein and optionally, wherein the cognate RNA-binding sequence is an RNA stem-loop sequence. In some embodiments, the detecting of steps f) and g) is performed using next generation sequencing. In some embodiments, the detecting of steps f) and g) is performed by i) reverse transcribing the mRNAs encoding the proteins of interest and comprising the RNA stem-loop; ii) performing a second strand synthesis on the reverse transcription product; iii) fragmenting the second strand synthesis product; iv) ligating nucleic acid linkers to the fragmented nucleic acids; v) amplifying the ligated nucleic acids; and vi) sequencing the amplified nucleic acids.

In some embodiments, the biochemical assay in an immunoprecipitation assay or a subcellular fractionation. In some embodiments, the plurality of different proteins of interest are representative of a proteome of interest. In some embodiments, the proteome of interest is the proteome of the cells. In some embodiments, the cells are Saccharomyces cerevisiae.

In some embodiments, the determining further comprises normalizing the amount of mRNA detected to the amount of mRNA detected of non-specific functional controls. In some embodiments, the non-specific functional controls are proteins of interest represented in the plurality of proteins of interest but are not isolated by the biochemical assay.

In certain aspects, the invention provides a method of determining protein-protein interactions, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest; d) lysing the population of cells; e) incubating the lysate of step d); f) performing proximity ligation to generate a plurality of hybrid sequences comprising the mRNA sequence encoding a first protein of interest and a mRNA sequence encoding one or more additional proteins of interest; g) for each protein of interest, sequencing the hybrid sequence generated in step f); for each protein of interest, h) identifying the one or more additional proteins of interest encoded by each hybrid sequence; wherein the additional proteins of interest of the plurality of hybrid sequences are identified as forming a protein-protein interaction with the first protein of interest.

In certain aspects, the invention provides a method of determining protein-protein interactions, the method comprising: a) providing a population of cells, wherein each cell comprises one or more nucleic acid sequences encoding: a RNA-binding protein; a protein of interest; and a nucleotide sequence encoding a unique molecular identifier (UMI) sequence operably linked to a nucleotide sequence encoding an RNA stem-loop, wherein the nucleic acid sequence encoding the RNA-binding protein and protein of interest are operably linked so that they encode a fusion protein of the RNA-binding protein and protein of interest; and wherein the nucleic acid sequence encoding the UMI and the RNA stem-loop are operably linked so that a mRNA encoding the protein of interest comprises the UMI sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds to the RNA stem-loop present on the RNA sequence encoding the UMI sequence and the RNA stem-loop sequence; d) lysing the population of cells; e) incubating the lysate of step d); f) performing proximity ligation to generate a plurality of hybrid sequences comprising the RNA sequence encoding a first UMI and a RNA sequence encoding one or more additional UMIs; g) for each hybrid sequence generated in step f), sequencing the one or more UMIs in the hybrid sequence; h) determining the protein of interest associated with each UMI of each hybrid sequence in g); wherein the proteins of interest associated with a hybrid sequence are identified as forming a protein-protein interaction.

In certain aspects, the invention provides a method of determining protein-DNA or protein-RNA interactions, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;

wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest; d) lysing the population of cells; e) adding a population of DNA or RNA molecules to the lysate of step d); f) incubating the mixture of lysate and a population of DNA or RNA molecules of step e); g) performing proximity ligation to generate a plurality of hybrid sequences comprising the mRNA sequence encoding a protein of interest or a cDNA copy thereof and one or more of the DNA or RNA molecules; h) for each protein of interest, sequencing the hybrid sequence generated in step g); i) for each protein of interest, identifying the one or more DNA or RNA molecules of each hybrid sequence; wherein the one or more DNA or RNA molecules of the plurality of hybrid sequences are identified as forming an interaction with the protein of interest.

In certain aspects, the invention provides a method of determining protein-DNA or protein-RNA interactions, the method comprising: a) providing a population of cells, wherein each cell comprises one or more nucleic acid sequences encoding: a RNA-binding protein; a protein of interest; and a nucleotide sequence encoding a unique molecular identifier (UMI) sequence operably linked to a nucleotide sequence encoding an RNA stem-loop; wherein the nucleic acid sequence encoding the RNA-binding protein and protein of interest are operably linked so that they encode a fusion protein of the RNA-binding protein and protein of interest; and wherein the nucleotide sequence encoding the UMI sequence and the RNA stem-loop are operably linked so that a mRNA encoding the RNA stem-loop comprises the UMI sequence;

wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds with high affinity to the RNA stem-loop on the nucleotide sequence encoding the UMI sequence and the RNA stem-loop; d) lysing the population of cells; e) adding a population of DNA or RNA molecules to the lysate of step d); f) incubating the mixture of lysate and a population of DNA or RNA molecules of step e); g) performing proximity ligation to generate a plurality of hybrid sequences comprising the mRNA sequence encoding the UMI or a cDNA copy thereof and one or more of the DNA or RNA molecules; h) for each hybrid sequence generated in step g), sequencing the UMI and the one or more DNA or RNA sequences of the hybrid sequence; i) determining the protein of interest associated with each UMI of each hybrid sequence in h); wherein the one or more DNA or RNA molecules of the plurality of hybrid sequences are identified as forming an interaction with the protein of interest.

In certain aspects, the invention provides a method of determining protein-DNA or protein-RNA interactions, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the protein of interest; d) lysing the population of cells; e) performing a biochemical assay on a portion of the lysate of step d) to generate an enriched lysate; wherein the biochemical assay comprises enriching a DNA or RNA molecule of interest; f) detecting the amount of mRNA encoding each protein of interest and comprising the RNA stem-loop that is present in the lysate of step d); g) detecting the amount of mRNA encoding each protein of interest and comprising the RNA stem-loop that is present in the enriched lysate of step e); and h) for each protein of interest, comparing the amount of mRNA detected in step d) and step g), wherein a protein of interest is determined to be enriched under the biochemical assay conditions if an increased level of mRNA is detected in step g) compared to step d) or wherein a protein of interest is determined to be depleted under the biochemical assay conditions is a decreased level of mRNA is detected in step g) compared to step d).

In certain aspects, the invention provides method of determining protein-DNA or protein-RNA interactions, the method comprising: a) providing a population of cells, wherein each cell comprises one or more nucleic acid sequences encoding: a RNA-binding protein; a protein of interest; a unique molecular identifier (UMI); and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the UMI comprises the cognate RNA sequence;

wherein the population of cells comprise nucleic acid sequences encoding a plurality of different proteins of interest; b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the UMI; d) lysing the population of cells; e) performing a biochemical assay on a portion of the lysate of step d) to generate an enriched lysate; wherein the biochemical assay comprises enriching a DNA or RNA molecule of interest; f) detecting the amount of mRNA encoding each UMI and comprising the RNA stem-loop that is present in the lysate of step d); g) detecting the amount of mRNA encoding each UMI and comprising the RNA stem-loop that is present in the enriched lysate of step e); and h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).

### BRIEF DESCRIPTION OF FIGURES

The patent or application file contains at least one drawing originally executed in color. To conform to the requirements for PCT patent applications, many of the figures presented herein are black and white representations of images originally created in color.

FIGS. 1A-E shows high throughput proteomics using in vivo mRNA display and next generation sequencing. (A) N-terminal MS2 capsid protein fused to a gene of interest binds the RNA stem-loop present on the 3′UTR of its encoding mRNA. (B) In vivo mRNA display libraries of the yeast ORFeome consist of a mixed population of strains, each expressing a single displayed protein interacting with its native cellular environment independently of other library species. (C) A proteomic assay with RNA sequencing as the readout. Scheme for a co-purification assay of a given bait from in vivo mRNA display extracts, whereby RNA is processed from both purified and the input lysate. Potential interactors are detected by comparing RNA read frequencies in the two samples for each displayed mRNA. (D) Log₂ Fold Enrichment of displayed mRNA for purified proteins compared to the lysate is calculated using quantitative PCR for a given construct with ACT1 as a reference. Two in vivo display constructs (MCP-mCherry, MCP-GFP) versus defective capsid constructs (MCP*-mCherry, MCP*-GFP) show significant relative enrichment (P=0.002, P=0.009, one-way ANOVA). (E) Log₂ Fold Enrichment of displayed mRNA for purified proteins from a mixed construct population (MCP-mCherry and MCP-GFP) for anti-RFP and anti-GFP purifications. The mRNA species of each specific protein is enriched relative to the input lysate over the non-specific species (RFP-IP: P=0.016, GFP-IP: P=0.001, t-test). For all purifications, cropped western blot images against RFP, GFP and α-Tubulin are shown to the left (for full images see FIG. 5). Biological replicates are represented as gray dots; bars represent mean signal.

FIGS. 2A-G shows that in vivo mRNA display enables precise protein identification in a complex mixture. (A) Assessment of in vivo mRNA display precision by identification of a specific protein subpopulation. Anti-FLAG immunoprecipitation from a mixed population containing HIS (yellow), MYC (green) and FLAG (blue) tagged yeast in vivo mRNA display constructs. Scatter plot for log normalized reads for the lysate (x-axis) against the purified samples (y-axis). Reads for each sample were normalized by the mean of non-specific functional controls. For each population, the area between a rolling 10^{th} and 90^{th} percentile is shaded with the respective color. (B) Display Score box plots for anti-FLAG, anti-MYC, and anti-HIS purifications. Shown are box plots of Display Scores for each subpopulation and the non-specific functional controls. The box extends from the lower to the upper quartile values, while whiskers extend 1.5×(Q3−Q1) outside the box and outliers are shown as individual points. (C) Receiver Operating Characteristic curves for the purifications in (B). Members of the mixed library were classified according to their respective Display Score. (D) Yeast in vivo mRNA display library purification. Average Display Scores were calculated per gene for over 3,300 ORFs over four biological replicates. Shown is a box plot for the ORFs in the library compared to the set of non-specific functional controls. (E) Volcano plot for the display score of the yeast library purifications. P-values were calculated with respect to the non-specific functional controls (see Methods, and q-values calculated using a Benjamini-Hochberg correction). (F) Scatter plot for Display Scores between two replicates (Pearson correlation is reported). (G) Percentage of in vivo mRNA display proteins with significant Display Scores per GO term biological process category.

FIGS. 3A-I shows that in vivo mRNA display enables high-throughput protein localization and interaction assays. (A-B) A crude mitochondrial isolation. (A) Display z Scores of the enrichment for individual mRNAs in a crude mitochondrial isolation. Replicates are denoted with circles while averages are reported as horizontal lines (z Scores were calculated with respect to the non-specific functional controls). TOM70, POR1, POR2, COX7, TIM23, IDH1, and PUT1 represent categories specific to the crude mitochondrial enrichment. LEU2, MPE1, ASN2, and SAM2 represent cytoplasmic and nuclear fractions. (B) Percentage of in vivo mRNA display proteins with significant Display Scores per GO term compartment category. Categories specific to the crude mitochondrial enrichment are shown starting with the endoplasmic reticulum and ending with the vacuole bars on the graph, while cytoplasmic and nuclear fractions are reported in the nucleus, cytosol, and cytoplasm categories. Organelle and membrane proteins are enriched while cytosolic proteins are significantly depleted (hypergeometric test for p-values). (C-I) Identifying Protein-Protein interactors for SAM2 and ARC40. (C-E) Co-Purification using anti-GFP magnetic beads from SAM2-GFP (C), ARC40-GFP (D) or control GFP (E). Experiments were performed in biological quadruplicates. Scatter plots are shown for the log normalized reads for the lysate (x-axis) against the purified samples (y-axis). The area between a rolling 10^{th} and 90^{th} percentile is shaded with the respective color. GFP mRNA is a positive control for the assay and is enriched in all three purifications. Hits for SAM2 and ARC40 are noted as black crosses. Grey dots denote non-specific ARC40 and SAM2 hits that are also significantly enriched in the GFP samples (common background). (F-G) Volcano plots for the display score for SAM2 (F) and ARC40 (G). P-values were calculated with respect to the non-specific functional controls (see Methods). (H) Volcano plot for mass-spectrometry of purified SAM2 and ARC40 samples (black crosses). The common hits for both MS and in vivo mRNA display for the two purified proteins are shown in yellow and blue respectively. The remainder MS hits are denoted in grey. (I) Display Z Scores for individual ARC40 interactors in a low-throughput purification of ARC40 (top) and SAM2 (bottom panel). Replicates are denoted with circles while averages are reported as horizontal lines. z Scores were calculated with respect to the non-specific functional controls (shown in black).

FIGS. 4A-D show that in vivo mRNA Display proteins co-purify their cognate mRNA for a variety of constructs and purification tags. (A-C) Log Fold Enrichments for purified proteins were calculated with respect to a reference (ACT1) in each purified sample and normalized to the construct with no hairpin loop. (A) MCP fusion constructs (mCherry) with no hairpin, one and two stem-loops (SLs). Samples with SLs display significantly more than the no-stem-loop sample. There is no significant difference between one and two stem-loops. (B) MCP-mCherry (red) and -GFP (green) constructs with no stem-loop, as well as their in vivo mRNA display counterparts, and defective MCP fusions (MCP*). While the presence of a SL allows for in vivo mRNA display in the MCP constructs, it has no effect for MCP*. (C) In vivo mRNA display is independent of purification tags used. Constructs (mCherry and GFP) with no stem-loop and a single hairpin purified with anti-HIS, -MYC, -FLAG, -RFP, and -GFP magnetic beads. (D) Similar to C, but Log Fold Enrichments were calculated with respect to a housekeeping gene in each purified sample and normalized to the lysate. qPCR averages are shown as bars and SD of technical replicates as errors.

FIG. 5 shows Western blots for FIGS. 1 D-E. Complete images of samples presented in FIGS. 1D-E. Samples were run on an SDS-PAGE gel and probed with GFP, RFP and α-Tubulin antibodies as described in Methods.

FIG. 6 shows a pipeline for high-throughput sequencing of in vivo mRNA library.

FIGS. 7A-B shows that restriction enzyme digestion generates a tighter distribution of fragment lengths for the yeast proteome. (A) Distribution of ORF lengths for the yeast proteome (B) distribution of 3′ and 5′ fragments after cDNA synthesis and RE digestion with the two enzyme mixes (top: AciI and HinP1I; bottom: MspI and HpyCH4IV). Histograms are plotted on the left, while cumulative distributions are plotted on the right.

FIG. 8 shows restriction enzymes in universal sequences flanking in vivo mRNA display ORFs. Introduction of additional cut sites flanking each ORF to ensure representation of every yeast protein during sequencing preparation.

FIGS. 9A-B show one-on-one completion of in vivo mRNA display constructs: scheme and sequencing preparation fragment enrichment. (A) Specific and non-specific mRNA for two construct purification experiments in FIG. 1E. (B) Bioanalyzer quantification of fragments from the two color competition in FIG. 1E. RNA libraries were prepared according to the sequencing pipeline. mCherry corresponding fragments are shown in red and GFP fragments in green. The top panel corresponds to the frequency of fragments from the lysate while the bottom corresponds to the purified sample. mCherry fragments are ˜8× enriched with respect to GFP fragments in agreement with the qPCR data.

FIG. 10 shows GFP purification from a library with 25 non-specific functional controls. GFP mRNA fragments are enriched in the purified sample compared to the non-specific functional controls and 7 flow-through control mRNAs. Boxplot distributions are shown. The box extends from the lower to the upper quartile values, while whiskers extend 1.5×(Q3−Q1) outside the box and outliers are shown as individual points.

FIG. 11 shows post-sequencing data analysis pipeline. See Methods for details.

FIG. 12 shows specificity concerns: mixed populations with constructs lacking a stem-loop. Log Fold Enrichment of specific over non-specific mRNA for purifications from mixed populations. Constructs with no hairpin loop are mixed with functional in vivo mRNA display constructs. In vivo display efficiency is lower in the presence of another functional construct (compare M4 to M3 and M8 to M7). Additionally, when no HL constructs are purified in the presence of functional constructs (M2 and M6), the non-specific mRNA is enriched which is an additional concern. qPCR averages are shown as bars and SD of technical replicates as errors.

FIG. 13 shows Excess Capsid or Stem-loop for increased display enrichment. In vivo mRNA display GFP constructs were mixed with defective capsid mCherry constructs and the Log Fold Enrichment of specific over non-specific mRNA was quantified for samples purified with anti-GFP magnetic beads. qPCR averages are shown as bars and SD of technical replicates as errors.

FIG. 14 shows that increasing temperature decreases precision of in vivo mRNA display assay. In vivo mRNA display GFP and mCherry constructs were mixed together and the Log Fold Enrichment of specific over non-specific mRNA was quantified for samples purified with anti-RFP (M1, M2) and anti-GFP (M3, M4) magnetic beads. For M1 and M3, samples were kept at 4° C. throughout purification. For M2 and M4, samples were incubated at 30° C. for 30 min post lysis. qPCR averages are shown as bars and SD of technical replicates as errors.

FIGS. 15A-C shows assessment of in vivo mRNA precision by purification of specific protein subpopulations. Anti-FLAG (A), anti-MYC (B), anti-HIS (C) immunoprecipitation from a mixed population containing HIS (yellow), MYC (green) and FLAG (blue) tagged yeast in vivo display sub-populations. Scatter plot for log normalized reads for the lysate (x-axis) against the purified samples (y-axis). Reads for each sample were normalized by the mean of non-specific functional controls. For each population, the area between a rolling 10th and 90th percentile is shaded with the respective color.

FIGS. 16A-H show distribution of reads for in vivo mRNA display yeast library purification. Distribution of log 2(reads+1) for every sample of the yeast library replicates (FIGS. 2D-G; Left: Lysates; Right: Purified IP samples). Total number of reads indicated in thousands for each sample. (A) Replicate 1 of lysate sample. (B) Replicate 2 of lysate sample. (C) Replicate 3 of lysate sample. (D) Replicate 4 of lysate sample. (E) Replicate 1 of IP sample. (F) Replicate 2 of IP sample. (G) Replicate 3 of IP sample. (H) Replicate 4 of IP sample.

FIG. 17 shows Lysate vs. Purified reads for in vivo mRNA display yeast library purification. Scatter plot for average log normalized reads for the lysate (x-axis) against the purified samples (y-axis) for the purified yeast library (FIGS. 2D-G). Reads for each sample were normalized by the mean of non-specific functional controls (grey crosses). GFP is an specific functional positive control (green cross). The area between a rolling 10th and 90th percentile is shaded with the respective color. Purified ORFs are enriched in the purified sample compared to the non-specific functional controls.

FIGS. 18A-F show display scores for in vivo mRNA display yeast library purifications are reproducible. Scatter plot for display scores between all yeast library purification biological replicates (Pearson and Spearman correlations reported). (A) Scatter plot for Display Scores between replicates 1 and 2. (B) Scatter plot for Display Scores between replicates 3 and 2. (C) Scatter plot for Display Scores between replicates 3 and 1. (D) Scatter plot for Display Scores between replicates 4 and 2. (E) Scatter plot for Display Scores between replicates 4 and 1. (F) Scatter plot for Display Scores between replicates 3 and 4.

FIGS. 19A-B show that in vivo mRNA display yeast library proteins span cellular compartments. Percentage of in vivo mRNA display yeast library proteins with significant Display Scores per GO term compartment category. (A) Percentage of the total number of genes in a GO Term category (orange: percentage of genes detected in the in vivo mRNA display assay and significantly displayed; yellow: percentage of genes detected in the in vivo mRNA display assay and not significantly displayed; grey: percentage of genes not detected in the in vivo mRNA display assay). (B) Percentage of the total genes detected in the in vivo mRNA display assay (orange: percentage of genes significantly displayed; yellow: percentage of genes not significantly displayed).

FIGS. 20A-B show that in vivo mRNA display yeast library proteins span biological processes. Percentage of in vivo mRNA display proteins with significant Display Scores per GO term biological process category. (A) Percentage of the total number of genes in a GO Term category (orange: percentage of genes detected in the in vivo mRNA display assay and significantly displayed; yellow: percentage of genes detected in the in vivo mRNA display assay and not significantly displayed; grey: percentage of genes not detected in the in vivo mRNA display assay). (B) Percentage of the total genes detected in the in vivo mRNA display assay (orange: percentage of genes significantly displayed; yellow: percentage of genes not significantly displayed).

FIGS. 21A-B show in vivo mRNA display yeast library proteins span molecular functions. Percentage of in vivo mRNA display proteins with significant Display Scores per GO term molecular function category. (A) Percentage of the total number of genes in a GO Term category (orange: percentage of genes detected in the in vivo mRNA display assay and significantly displayed; yellow: percentage of genes detected in the in vivo mRNA display assay and not significantly displayed; grey: percentage of genes not detected in the in vivo mRNA display assay). (B) Percentage of the total genes detected in the in vivo mRNA display assay (orange: percentage of genes significantly displayed; yellow: percentage of genes not significantly displayed).

FIGS. 22A-B show crude mitochondrial isolation volcano plot and read distributions. (A) Volcano plot for the crude mitochondrial purification replicates. Average display score (x-axis) against q-values (p-values were calculated with respect to the non-specific functional controls and Benjamini-Hochberg corrected; see Methods). (B) Distribution of log 2(reads+1) for every sample of the crude mitochondrial subfractionation (Left: Supernatant; Right: Crude Mitochondrial Pellets). Total number of reads indicated in thousands for each sample.

FIG. 23 shows receiver operating characteristic curves for the crude mitochondrial isolation in FIGS. 3A-B. Members of the library were classified according to their respective Display Score and compared to the GO Term compartment categories (FIG. 3B). ROC curves for individual replicates are shown in the bottom panel.

FIGS. 24A-C show enrichment of organelle and membrane categories compared to cytosolic proteins. (A) from GO Term categories (B) localization DB, and (C) high throughput mitochondrial study. P-values for enrichments and depletions were calculated using the hypergeometric test between the number of ORFs with significant Display Scores in each category compared to the significant Display Scores present in the assay.

FIGS. 25A-H show distribution of reads for in vivo mRNA display yeast library SAM2 purification. Distribution of log (reads+1) for every SAM2 purification in FIG. 3C. Total number of reads noted in thousands for each sample. (A) Replicate 1 of lysate sample. (B) Replicate 2 of lysate sample. (C) Replicate 3 of lysate sample. (D) Replicate 4 of lysate sample. (E) Replicate 1 of IP sample. (F) Replicate 2 of IP sample. (G) Replicate 3 of IP sample. (H) Replicate 4 of IP sample.

FIGS. 26A-H show distribution of reads for in vivo mRNA display yeast library ARC40 purification. Distribution of log (reads+1) for every ARC40 purification in FIG. 3D. Total number of reads noted in thousands for each sample. (A) Replicate 1 of lysate sample. (B) Replicate 2 of lysate sample. (C) Replicate 3 of lysate sample. (D) Replicate 4 of lysate sample. (E) Replicate 1 of IP sample. (F) Replicate 2 of IP sample. (G) Replicate 3 of IP sample. (H) Replicate 4 of IP sample.

FIGS. 27A-J show distribution of reads for in vivo mRNA display yeast library negative control purifications. Distribution of log (reads+1) for every control purification in FIG. 3E. Total number of reads noted in thousands for each sample. (A) Replicate 1 of lysate sample. (B) Replicate 2 of lysate sample. (C) Replicate 3 of lysate sample. (D) Replicate 4 of lysate sample. (E) Replicate 5 of lysate sample. (F) Replicate 1 of IP sample. (G) Replicate 2 of IP sample. (H) Replicate 3 of IP sample. (I) Replicate 5 of IP sample. (J) Replicate 5 of IP sample.

FIG. 28 shows a proteomic assay for identification of RNA or DNA interacting proteins with NGS sequencing as the readout.

FIGS. 29A-F show that in vivo mRNA display proteins co-purify a fraction of their cognate mRNA. Percentage of protein and mRNA in the flow-through and purified fractions with respect to the levels in the input sample. A single step purification was tested for two HIS-tagged (A, B, D, and E) and one FLAG-tagged (C and F) construct using reduced salt concentration to avoid excessive loss of RNA and protein. Percentages of protein and RNA levels were calculated with respect to the total input whole cell extract for each strain (using fluorescence and qPCR respectively). Tagged protein constructs were purified in similar amounts with specificity independent of the presence of a stem loop (left-most column). To assess the percentage of cognate mRNA co-purified during the isolation process, we quantified RNA levels of specific and background RNA for constructs with and without stem loops in their 3′UTR. For all three panels, the MCP fusion constructs with no stem-loop co-purified similar levels of construct specific and ACT1 mRNA. In contrast, for the MCP constructs with a stem-loop, purification of the protein resulted in enriched construct specific mRNA with respect to both the reference and the construct with no stem loop. In addition, for the MCP constructs with a stem-loop, construct specific mRNA was depleted in the first flow-through (unbound fraction) with respect to the references. (A, D) anti-HIS purification of MCP-mCherry fusion construct with no stem-loop (HIS tag), one stem loop (HIS tag) and a no HIS tag construct. ˜30% of isolated protein co-purified ˜25% of mRNA carrying a stem loop. However, ˜10% of background RNA with no SL is also co-purified, resulting in an excess ˜15% that can be specifically attributed to stem loop binding. Therefore, it is estimated that ˜30% of isolated protein specifically co-purified ˜15% of its cognate mRNA. This percentage of RNA amounts to ˜%50 of the RNA that proportionally corresponds to the purified protein. (B, E) anti-HIS purification of MCP-GFP fusion constructs with no stem-loop (HIS tag), one stemloop (HIS tag) and no HIS tag constructs. ˜36% of isolated protein co-purified ˜25% of mRNA carrying a stem loop. However, ˜10% of background RNA with no SL is also co-purified, resulting in an excess ˜15% that can be specifically attributed to stem loop binding. Therefore, it is estimated that ˜36% of isolated protein specifically co-purified ˜15% of its cognate mRNA. This percentage of RNA amounts to ˜%40 of the RNA that proportionally corresponds to the purified protein. (C, F) anti-FLAG purification of MCP-GFP fusion constructs with no hairpin (FLAG tag), one stemloop

(FLAG tag) and no FLAG tag constructs. ˜40-45% of isolated protein co-purified ˜10% of mRNA carrying a stem loop. However, background RNA with no SL is co-purified in small amounts (<0.05%), resulting in a specific co-purification. Therefore, we estimate that ˜4045% of isolated protein specifically co-purified ˜10% of its cognate mRNA. This percentage of RNA amounts to ˜%20 of the RNA that proportionally corresponds to the purified protein.
Overall, it is estimated that isolated protein co-purifies roughly 20-50% of its corresponding mRNA with specificity. At the same time, while ˜80% of the displayed protein is missing from the flowthrough, the construct specific RNA is depleted in an excess of 20-40% compared to both a no SL control and a housekeeping reference gene. The protein and RNA that is not present in the first flow-through will either be purified or removed during the wash steps. Protein levels were assayed by means of fluorescence using a plate reader (Synergy MX, BioTek). Here, protein constructs were bound using the respective magnetic beads and washed with a reduced salt Wash Buffer (150 mM NaCl). RNA was precipitated from every sample using TRIzol in order to avoid inconsistent losses on the spin columns used otherwise in this manuscript. RNA levels were assessed using relative standard curves for each primer set (for mCherry, GFP, ACT1). Percentages of protein and RNA levels were calculated with respect to the total input whole cell extract for each strain.

FIG. 30 shows percentage of in vivo mRNA display proteins with significant Display Scores for proteins with signal and transit peptides. Shown in red are proteins that contain a N-terminal Signal peptide (UniProt annotation), or a Transit peptide (UniProt annotation), or membrane proteins (GO term: 16020). Cytoplasmic and nuclear fractions are reported in grey for reference. Membrane proteins are enriched at an overall higher percentage than proteins carrying peptides responsible for transport, which are usually cleaved from the mature protein and could interfere with the function of the MS2 N-terminal fusion (hypergeometric test for p-values).

FIGS. 31 A-C show that mammalian in vivo mRNA display proteins co-purify their cognate mRNA. (A) Log 2 Fold Enrichments of displayed mRNA for purified proteins expressed in human cells were calculated with respect to the input lysate in each purified sample and normalized to the construct with no hairpin loop. An in vivo mRNA display construct (MCP-acGFP with a cognate stem loop, 2 replicates) shows significant relative enrichment in contrast to defective coat protein construct (MCP*-acGFP with a cognate stem loop). (B) Similar to A but Log 2 Fold Enrichments of displayed mRNA for purified proteins expressed in human cells were calculated with respect to the input lysate of each purified sample and normalized to a reference gene (ACTB). An in vivo mRNA display construct (MCP-acGFP with a cognate stem loop, 2 replicates) shows significant relative enrichment in contrast to the same construct without a cognate stem loop. (C) A population of human cells expressing an in vivo mRNA display protein (MCP-acGFP with a cognate stem loop) mixed with cells expressing a construct lacking a stem-loop (MCP-mCherry). When the acGFP protein was purified, in vivo displayed acGFP mRNA was enriched with respect to the input lysate relative to non-displayed mCherry mRNA. qPCR averages are shown as bars and SD of technical replicates as error. All purifications were performed using anti-GFP beads (ChromoTek gtma).

FIGS. 32A-F show functional characterization of proteins using mutagenized in vivo mRNA display libraries. Co-purification of mutagenized ARC35 in vivo display library using anti-GFP magnetic beads for ARC40-GFP. The experiment was performed in biological replicates containing independently mutagenized libraries. (A, D) Non-synonymous mutations (yellow) are significantly more likely to have elevated depletion scores compared to synonymous mutations (grey). (B, E) Histogram of observed depleted and non-depleted nonsense mutations across all amino acid positions of ARC35 (amino acids 1-342). All detected nonsense mutations occurring before or within the functional domain of ARC35 (amino acids 1-316) were significantly depleted (purple) in the purified sample compared to the lysate. A small number of nonsense mutations that were not depleted (grey) occurred at the C-terminus of the protein that is outside the functional domain. (C, F) Histogram of all observed non-synonymous mutations across all amino acid positions of ARC35. All non-synonymous mutations are plotted in grey while depleted non-synonymous mutations are plotted in purple. The majority of non-synonymous mutations (grey) did not affect ARC35 participation in the complex, while ˜7% of such mutations show substantial depletion (purple) indicating a functional effect.

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The terms “animal,” “subject” and “patient” as used herein includes all members of the animal kingdom including, but not limited to, mammals, animals (e.g., cats, dogs, horses, swine, etc.) and humans.

As used herein, the term MS2 capsid and the term MS2 coat protein (MCP) are interchangeable.

In Vivo mRNA Display

At the global scale, mapping and understanding the astronomically complex network of molecular interactions among the millions of distinct components in the cell has become a major long-term goal for the field of systems biology. For example, there are some ˜10⁹ possible pair-wise interactions in the human proteome (even ignoring differential splice variants). This number grows to ˜10¹³ for all potential human DNA-protein interactions at 15 bp resolution. In a perfect world, a technology would allow us to efficiently, systematically, and quantitatively, measure all these potential interaction strengths, providing an unbiased architectural view. The advent of the yeast two-hybrid technology⁴ more than two decades ago, initiated an effort to move in this direction. Two-hybrid technology and its many variants, enable detection of protein-protein interactions by coupling a physical interaction between two interacting proteins to transcriptional activity of a reporter gene within the nucleus. This is achieved by fusing a DNA-binding domain to one protein, and a transcriptional activation domain to another. Physical interactions between the two proteins brings the DNA-binding domain to a specific DNA binding-site near a reporter gene. The transcriptional activation domain, in turn, activates the expression of the reporter gene. A major innovation of this technology was the generation of yeast libraries of ‘bait’ and ‘prey’ proteins, in opposite mating type haploid strains. Through robotic manipulations, one could perform automated mating of any bait-fusion yeast strain to an entire library of prey fusions. The interaction of a bait with a prey would enable the corresponding diploid yeast to grow into a visible colony, reporting the interaction in a binary fashion. Interactome maps generated by comprehensive two-hybrid assays have yielded highly valuable knowledge infrastructure in organisms ranging from yeast to human^{4,5}. In addition, slight variations in the technology allows analysis of DNA-protein interactions, called one-hybrid, and RNA-protein interactions, called three-hybrid.

The last decade has also seen the development of many alternative technologies to the Y2H. For example, protein fragment complementation assay (PCA) detects protein-protein interactions by reconstituting the function of an enzyme or fluorescent protein through the physical bridging of its two fragments by the interacting proteins⁶. In another technology, called MAPPIT⁷, the two components of a mammalian cytokine receptor are reconstituted upon an interaction, leading to the activation of downstream signaling⁷. A biochemical alternative to Y2H and its conceptually similar variants, is tandem affinity purification followed by mass spectrometry⁸. In this approach, a protein of interest is immunoprecipitated, using either an antibody to the protein or more commonly, to a recombinant tag fused to it. After purification, the co-immunoprecipitated proteins are identified by mass spectrometry⁸. If one wishes to query a particular interaction, the fusion of a luciferase enzyme to the query protein can allow detection through a simple enzymatic assay following immunoprecipitation. This approach, called LUMIER⁹, bypasses mass-spectrometry, and can be established in a medium throughput format.

The Y2H technology and its many variants have significantly improved our ability to detect bi-molecular interactions. More recently, large-scale efforts have attempted to systematically test a large number of possible protein-protein interactions in order to create a global interactome map. However, the requirement for testing individual interactions, through isolated macroscopic colony growth, requires extensive robotics infrastructure, operating on the timescale of years in order to create an interactome. More importantly, the nature of how a physical interaction is converted to reporter activity, inside the yeast nucleus, imposes significant biases that limit the sensitivity and specificity of Y2H. Because of these limitations, a Y2H operation, will take years to generate an interactome map, and end up covering only a few percent of all possible human protein-protein interactions. The biochemical alternatives to Y2H and PCA are even more labor-intensive, costly, and have lower throughput. Despite all these limitations, these partial interactome maps have been quite valuable to biomedical scientists, providing insights that have uniquely shaped our systems-level understanding of protein-protein interaction networks. However, biology is in critical need of next-generation methods that substantially increase throughput and coverage, and decrease cost, such that full-coverage protein interactome maps can be generated for the hundreds of organisms of basic and medical interest. Furthermore, there is a critical need for technologies that extend these observations to the world of protein-DNA and protein-RNA interactions. High-throughput technologies to map protein-RNA interactions has been a particularly neglected area, especially given our increasing appreciation of the role of RNA-binding proteins in a variety of post-transcriptional processes controlling gene expression.

Application of next generation sequencing technologies to proteomic analysis allows high-throughput characterization of protein expression for a variety of research, diagnostic and therapeutic applications.

In certain aspects, the invention described herein relates to a platform for high-throughput proteomic analysis in vivo based on mRNA display. The technique associates expressed proteins with their own mRNA or a mRNA encoding a unique molecular identifier (UMI), allowing in vivo quantitative analysis of protein expression by DNA sequencing, using only standard lab equipment and a Next Generation Sequencing platform. In some embodiments, the platform used to perform DNA sequencing is Illumina sequencing although the invention is not limited to the Illumina sequencing platform. A variety of next generation sequencing platforms are known in the art, any of which can be used with the present invention to perform the DNA sequencing step of the inventive method. The technology allows analysis of protein expression levels and subcellular characterization in their relevant cellular context. This technology can be used for proteomic analysis in vivo as demonstrated in S. cerevisiae yeast and mammalian cells. This technology can also be used for proteomic analysis in bacterial cells, insect cells, human cell lines, mammalian cell lines, or animal models. This approach can be used to rapidly identify proteins in functional assays in vivo using next generation sequencing.

The in vivo mRNA display described herein is a novel technology for in vivo proteomics. It converts a variety of proteomics applications into a DNA sequencing problem by linking functional proteins to self-identifying nucleic acids in vivo. In vivo expressed proteins are coupled with mRNA sequences via a high-affinity stem-loop RNA binding domain interaction, enabling high-throughput identification of proteins with high sensitivity and specificity by next-generation DNA sequencing of the bound mRNA molecules. In vivo mRNA display libraries promise to circumvent the limitations of mass spectrometry-based proteomics and leverage the exponentially improving cost and throughput of DNA-sequencing to systematically characterize native functional proteomes. The invention facilitates parallel discovery and quantification of physical interactions between proteins and proteins, proteins and DNA, and proteins and RNA.

In certain aspects, described herein is a method for performing mRNA display proteomic analysis in vivo. The technology uses a modified MS2 tagging system to associate a translated protein with its own mRNA or with a mRNA encoding a UMI, increases throughput and reduces cost compared to conventional spectrometry-based proteomics. In vivo protein tagging allows analysis of protein expression levels, subcellular characterization, and interaction with binding partners in their relevant cellular contexts, potentially reducing artifacts associated with cell lysis. The technology has demonstrated high-throughput characterization of the yeast ORFeome, capturing ˜3400 proteins in the mRNA display library. The technology is not limited to yeast cells, and has also demonstrated high-throughput characterization of proteins in mammalian cells. This technology also can be used for high-throughput characterization of proteins in bacterial cells, insect cells, human cell lines, mammalian cell lines, or animal models.

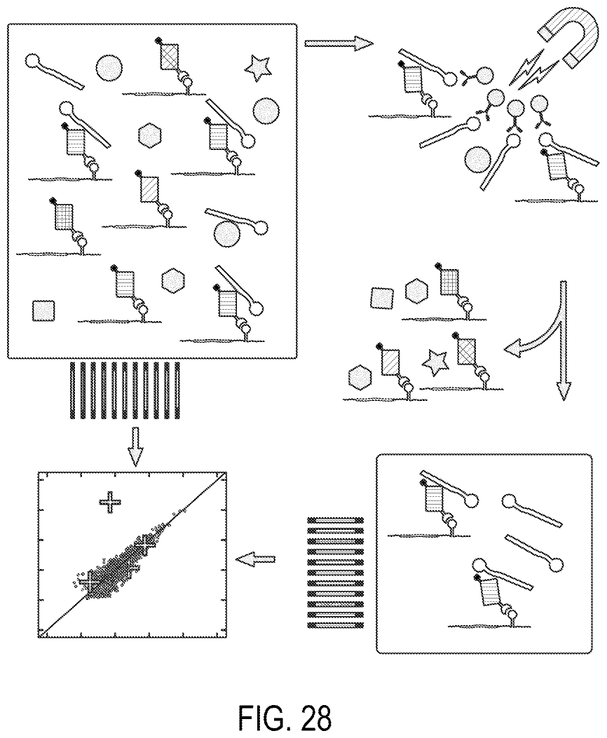

In some embodiments, the subject matter disclosed herein is utilized in in vivo proteomic sequencing. In some embodiments, the subject matter disclosed herein is utilized in measurement of all protein-protein interactions in an organism. In some embodiments, the subject matter disclosed herein is utilized in massively parallel measurements of interactions between proteins and nucleic acids, including DNA and RNA. In some embodiments, the subject matter disclosed herein is utilized in characterization of protein binding domains in vivo. In some embodiments, the subject matter disclosed herein is utilized as a research tool for protein evolution, in vivo biopanning, and protein engineering. An embodiment of a co-purification assay of a given DNA or RNA bait from in vivo mRNA display extracts, whereby RNA is processed from both purified and the input lysate, is shown in FIG. 28. RNA or DNA baits can be expressed in vivo with an aptamer or other affinity handle, or they can be incubated ex vivo with library lysate. In some embodiments, potential interactors are detected by comparing RNA read frequencies in the two samples for each displayed mRNA.

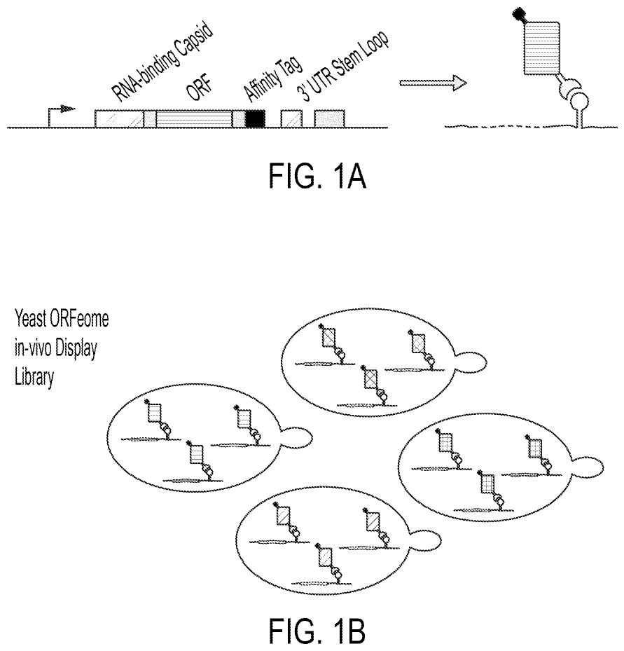

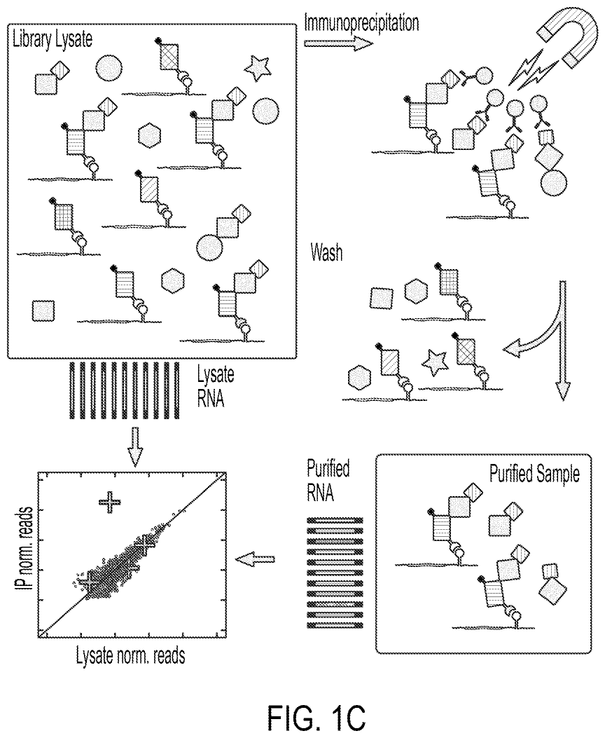

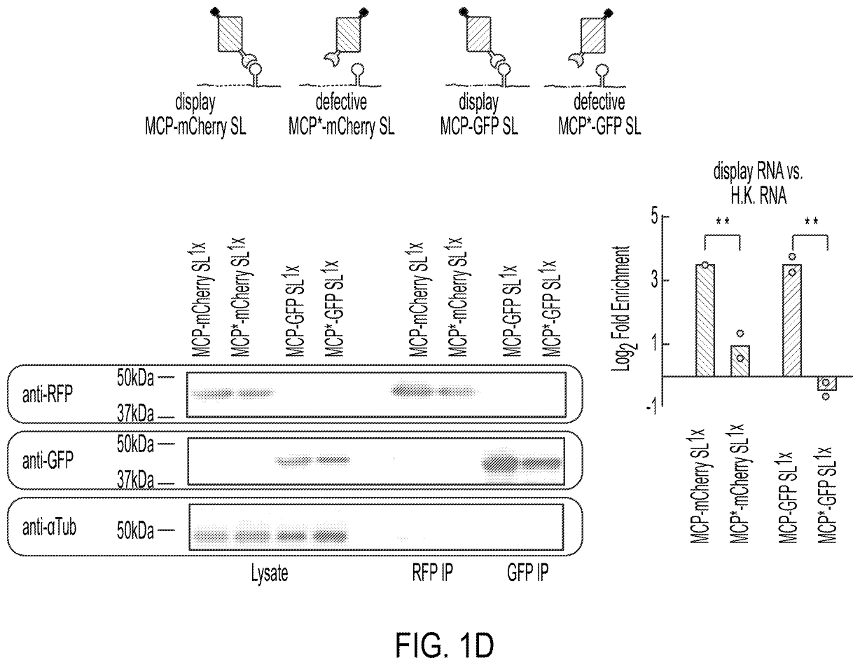

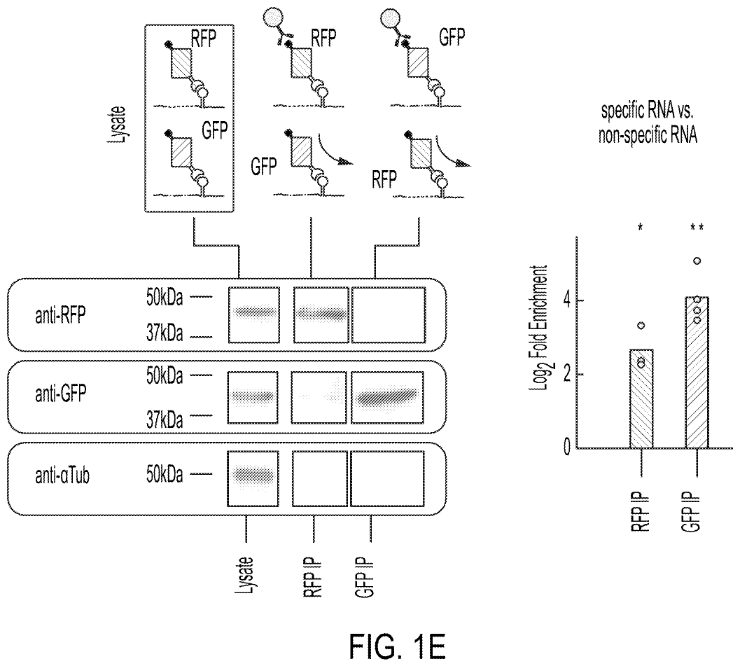

In certain aspects, the subject matter disclosed herein relates to generating in vivo mRNA display proteins. The high-affinity interaction between the MS2 bacteriophage capsid protein and its cognate RNA stem-loop was co-opted. The MS2 tagging system was modified in order to associate a translated protein with its own mRNA. The MCP was fused to the N-terminus of a target protein while the cognate RNA hairpin was introduced downstream of the gene establishing a direct link between gene and protein (FIG. 1A). In an alternative embodiment, the MS2 tagging system is modified in order to associate a translated protein with a mRNA encoding a UMI. The MCP is fused to the N-terminus of a target protein while the cognate RNA hairpin is operably linked to a UMI establishing a direct link between each UMI and protein. In some embodiments, the RNA binding domain is PP7 bacteriophage coat protein, which recognizes its cognate RNA hairpin. In some embodiments, the RNA binding domain is the 22 amino acid RNA-binding domain of the lambda bacteriophage antiterminator protein N (lambdaN-(1-22) or lambdaN peptide), which binds to its specific 19 nucleotide binding site (boxB) RNA sequence. In some embodiments, the subject matter disclosed herein utilizes any suitable bacteriophage RNA binding system. In contrast to in vitro display technologies, assayed proteins are expressed, processed, and tagged in vivo in their relevant cellular contexts. This approach, termed in vivo mRNA display, identifies proteins in a variety of in vivo functional assays using nucleic-acid sequencing as the readout.

In certain aspects, the subject matter disclosed herein relates to a population of cells, wherein each cell contains a single species of the in vivo mRNA display construct corresponding to a single displayed protein, which interacts with its cellular context independently from all the other species in the library (FIG. 1). Induced cells can be assayed according to the desired biochemical assay (e.g. immunoprecipitation of a bait, subcellular fractionation etc.) which should preserve the RNA-protein interaction (FIG. 1C). In any given sample in vivo mRNA display proteins can be quantified by measuring the abundance of their mRNA.

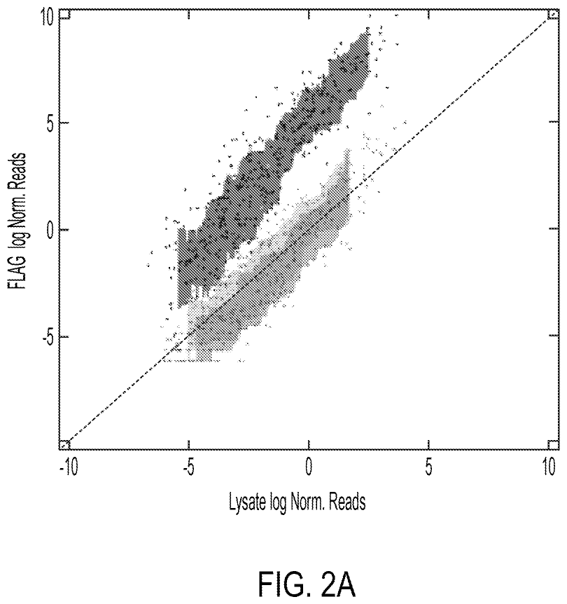

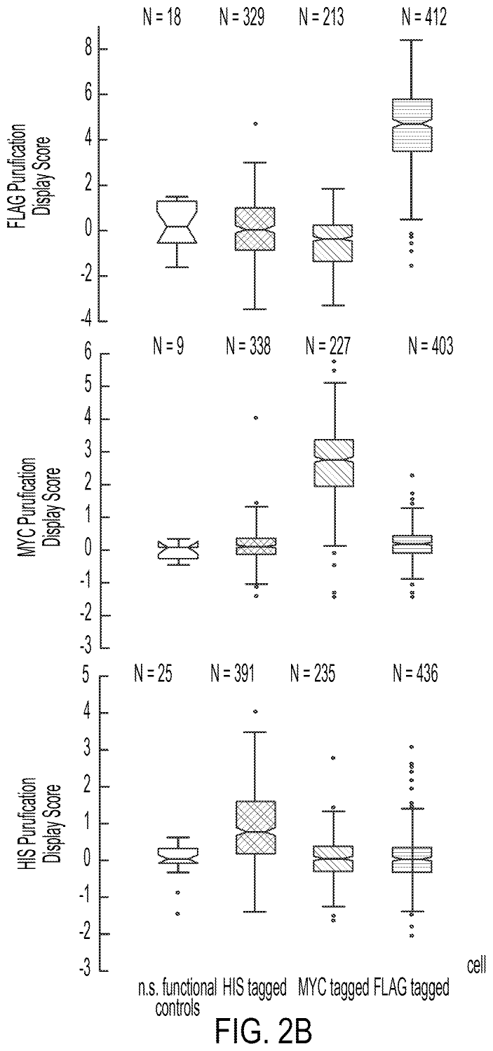

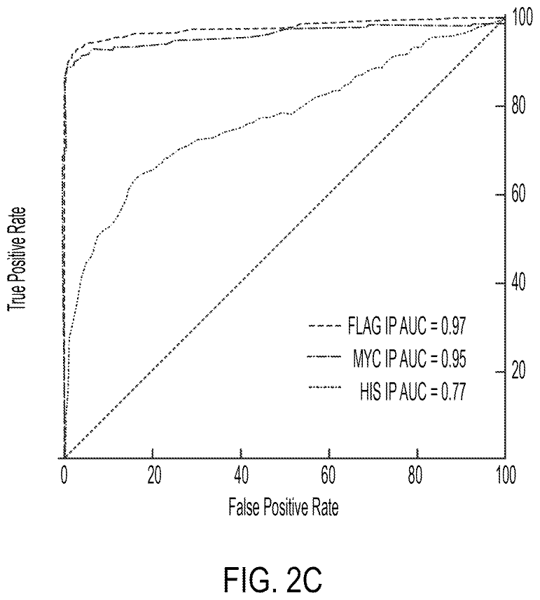

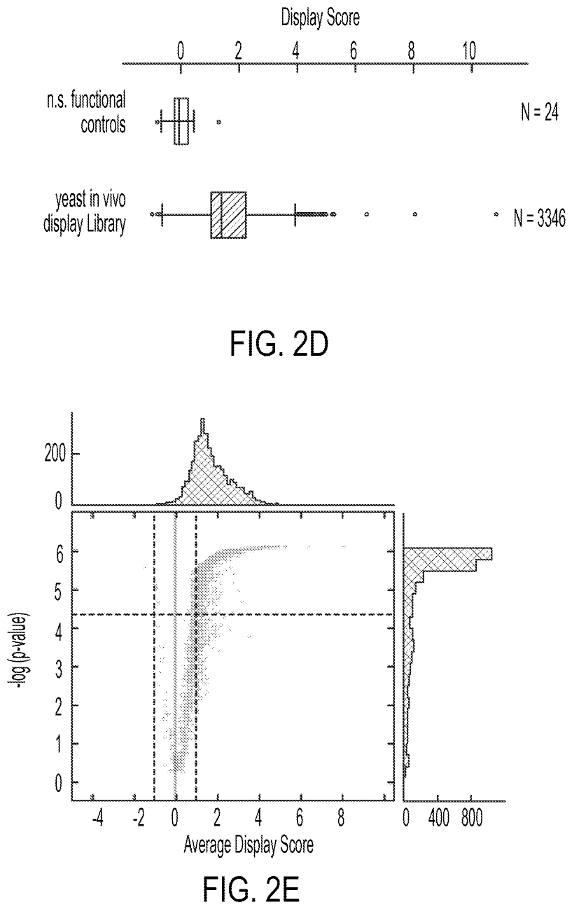

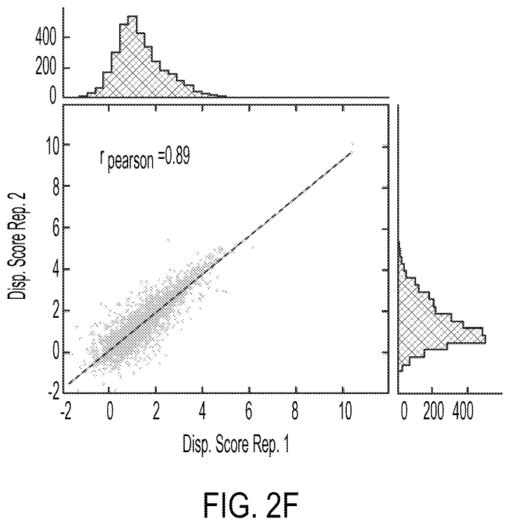

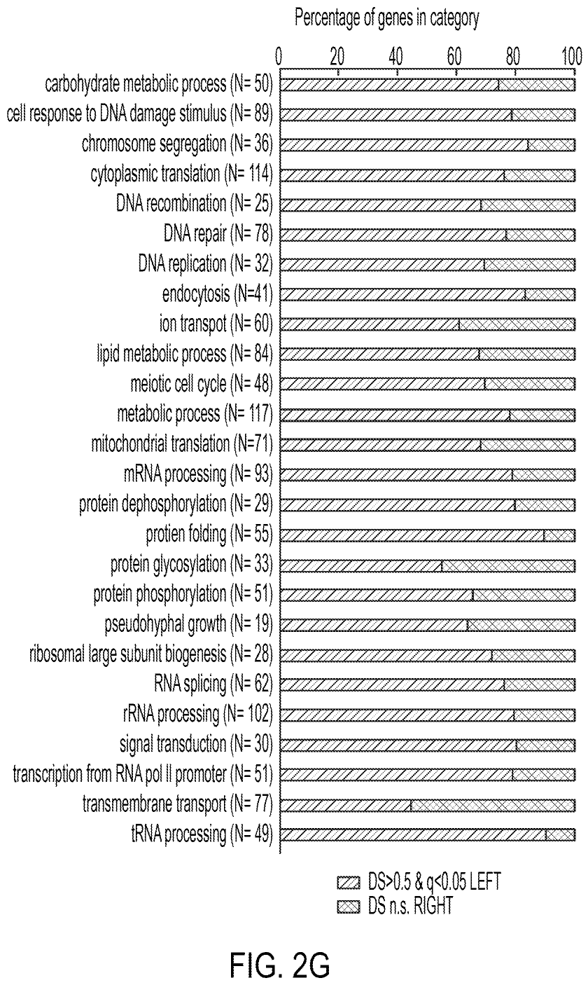

In some embodiments, there are four major advancements enabled by the invention described herein. First, the demonstration of a display technology in vivo and the use of sequence based quantification as the readout for proteomics. In vivo mRNA display proteins can be isolated and correctly identified by comparing the enrichment of their mRNA levels to reference mRNA species (FIG. 1D) and to other proteins (FIG. 2A) with high precision. Proteins can be quantified by means of Next Generation Sequencing, Quantitative qPCR, electrophoresis, etc.

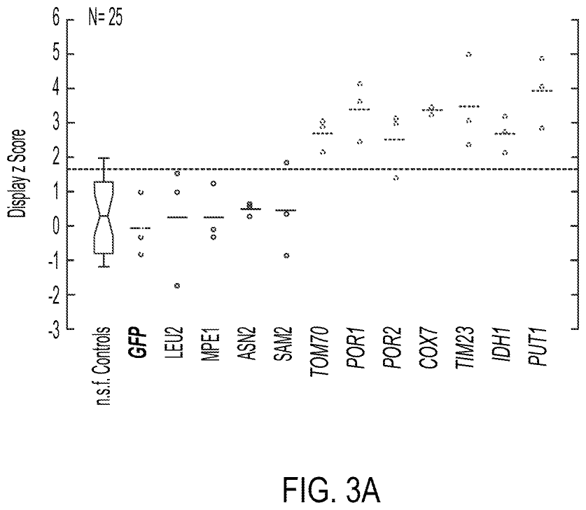

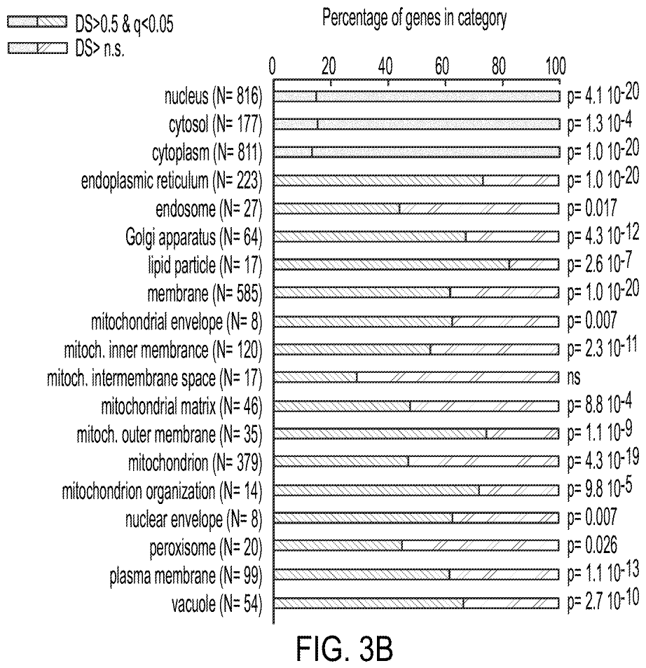

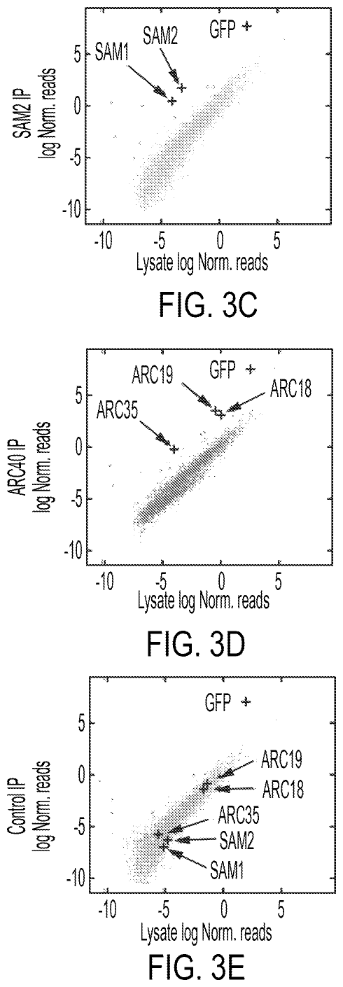

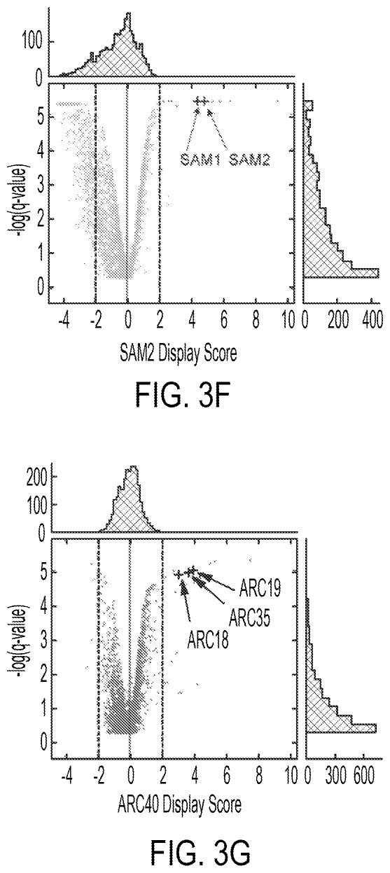

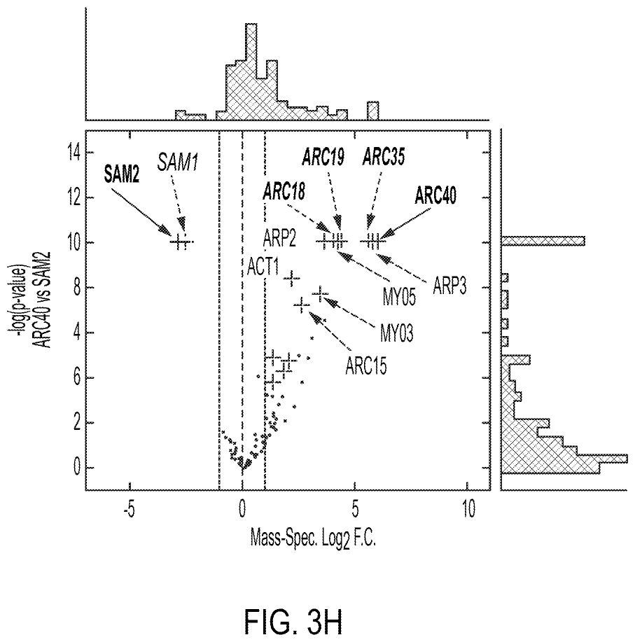

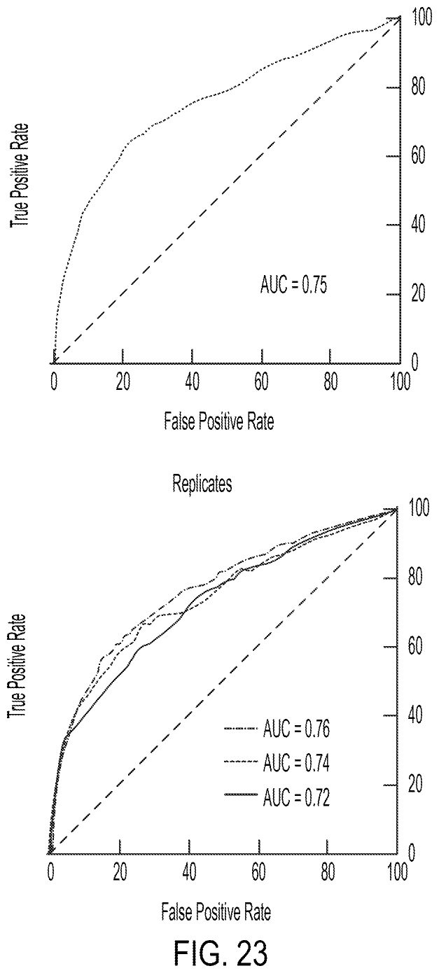

Second, the invention described herein can be used for high-throughput characterization of whole proteomes in vivo. To this end, an in vivo mRNA display library of the yeast ORFeome was constructed. Protocols for handling and processing in vivo mRNA display libraries, preparation of isolated RNA for NGS sequencing, and statistical measures for protein quantification were developed. Isolated RNA can be processed with RNA-seq preparation methods known in the art. The library captured ˜3400 proteins. The techniques can capture the native sub-cellular compartmentalization of the yeast proteome, thus enabling systematic localization assays. In a crude mitochondrial isolation, in vivo mRNA display can capture proteins known to localize in the mitochondria and other organelles, as expected, while cytosolic and nuclear proteins are depleted (FIG. 3B). FIG. 23 shows receiver operating characteristic curves for the crude mitochondrial isolation in FIGS. 3A-B. Additionally, in vivo mRNA display can correctly identify the in vivo interaction partners of specific proteins of interest (FIGS. 3C-E).

Thirdly, this techniques is easily transferable to other organisms, as MS2 tagging can be used as reporter system to track mRNA molecules in living cells in a variety of organisms, including, but not limited to mammalian cells. Additionally, an in vivo mRNA display protein can be tagged with any RNA molecule unique molecular identifier (UMI), not just its cognate mRNA.

Fourthly, the invention described herein allows for the study of proteins with single nucleotide resolution in vivo. This includes studying the functional differences of single nucleotide variants by generating libraries of in vivo mRNA display proteins for such variants, and using in vivo mRNA display for directed protein evolution and biopanning. The source of the in vivo mRNA display peptides could be any biological or artificial Open-Reading-Frame (ORF) library.

In vivo mRNA display can be used as a novel method for proteomics, but can also be used for in vivo protein engineering, the functional study of protein domains, in vivo biopanning selections, and engineering of novel peptides for industrial or therapeutic purposes.

SEQ ID NO: 1 is the nucleotide sequence encoding the MS2 coat protein (MCP):

ATGCTAGCCGTTAAAATGGCTTCTAACTTTACTCA
GTTCGTTCTCGTCGACAATGGCGGAACTGGCGACG
TGACTGTCGCCCCAAGCAACTTCGCTAACGGGATC
GCTGAATGGATCAGCTCTAACTCGCGTTCACAGGC
TTACAAAGTAACCTGTAGCGTTCGTCAGAGCTCTG
CGCAGAATCGCAAATACACCATCAAAGTCGAGGTG
CCTAAAGGCGCCTGGCGTTCGTACTTAAATATGGA
ACTAACCATTCCAATTTTCGCCACGAATTCCGACT
GCGAGCTTATTGTTAAGGCAATGCAAGGTCTCCTA
AAAGATGGAAACCCGATTCCCTCAGCAATCGCAGC
AAACTCCGGCATCTAC

SEQ ID NO: 2 is the nucleotide sequence encoding the cognate RNA stem-loop for the MS2 capsid: gcacgAgcATCAgccgtgc. The lowercase bases can pair with each other to form the stem. The uppercase ATCA sequence can form the loop. The single uppercase A nucleotide is an unpaired bulge in the RNA stem-loop. SEQ ID NO: 3 is one embodiment of the RNA stem-loop for the MS2 capsid incorporated into a portion of a vector sequence:

ATCCTACGGTACTTATTGCCAAGAAAgcacgAgcA
TCAgccgtgcCTCCAGGTCGAATCTTCAAA

SEQ ID NO: 4 is the codon optimized sequence of the MS2 coat protein (MCP), optimized for expression in human cells:

ATGTTGGCGGTAAAGATGGCTTCTAACTTTACGCA
GTTCGTTCTCGTAGACAATGGCGGGACTGGGGACG
TAACAGTCGCCCCATCTAATTTTGCTAATGGAATA
GCGGAGTGGATAAGCAGTAATAGCCGAAGCCAGGC
CTATAAGGTGACATGCTCCGTGCGACAATCCAGTG
CTCAAAATCGAAAATACACCATTAAAGTAGAAGTC
CCTAAGGGCGCCTGGCGATCCTACCTTAACATGGA
GCTCACTATTCCAATCTTTGCTACCAATTCTGACT
GCGAGCTGATAGTAAAAGCAATGCAGGGTCTTTTG
AAGGACGGCAACCCGATTCCGTCCGCTATTGCTGC
AAATAGCGGGATTTAC

### Nucleic Acids of the Invention

In certain aspects, the invention provides a nucleic acid comprising a mRNA display cassette, the mRNA display cassette comprising a cloning site for insertion of a nucleotide sequence encoding a protein of interest operably linked to (i) a nucleotide sequence encoding a MS2 bacteriophage coat protein (MCP) and (ii) to a nucleotide sequence encoding an RNA stem-loop, wherein the MCP binds to the RNA stem-loop with high-affinity.

The invention is not limited to MCP and its cognate RNA sequence. Accordingly, in certain aspects, the invention provides a nucleic acid comprising a mRNA display cassette, the mRNA display cassette comprising a cloning site for insertion of a nucleotide sequence encoding a protein of interest operably linked to (i) a nucleotide sequence encoding a RNA binding protein and (ii) to a nucleotide sequence encoding an cognate RNA sequence, wherein the RNA binding protein binds to the cognate RNA sequence with high-affinity.

In some embodiments, the nucleotide sequence encoding the MCP or RNA binding protein is located 5′ to the cloning site for insertion of the nucleotide sequence encoding the protein of interest. In some embodiments, the nucleotide sequence encoding the MCP or RNA binding protein is located 3′ to the cloning site for insertion of the nucleotide sequence encoding the protein of interest. In some embodiments, the nucleotide sequence encoding the RNA stem-loop is located 3′ to the cloning site for insertion of the nucleotide sequence encoding the protein of interest. In some embodiments, the nucleotide sequence encoding the RNA stem-loop or cognate RNA sequence is located in a 3′ UTR. In some embodiments, the nucleotide sequence encoding the RNA stem-loop or cognate RNA sequence is located 5′ to the nucleotide sequence encoding the MCP or RNA binding protein.

In some embodiments, the mRNA display cassette is configured so that upon insertion of a nucleotide sequence encoding a protein of interest, the nucleotide sequence encoding the protein of interest and the MCP or RNA binding protein are operably linked so that they encode a fusion protein of the protein of interest and the MCP or RNA binding protein. In some embodiments, the fusion protein comprises the MCP or RNA binding protein fused to the N-terminus of the protein of interest. In some embodiments, the fusion protein comprises the MCP or RNA binding protein is fused to the C-terminus of the protein of interest.

In some embodiments, the mRNA display cassette further comprises a nucleotide sequence encoding a purification tag operably linked to the cloning site for insertion of the nucleic acid sequence encoding the protein of interest. In some embodiments, the mRNA display cassette is configured so that upon insertion of a nucleotide sequence encoding a protein of interest, the nucleotide sequence encoding the protein of interest and the purification tag are operably linked so that they encode a fusion protein of the protein of interest and the purification tag. In some embodiments, the fusion protein comprises the purification tag fused to the C-terminus of the protein of interest. Purification tags are well known in the art.

In some embodiments, the nucleic acid further comprises a promoter operably linked to the mRNA display cassette. In some embodiments, the promoter is an inducible promoter. Promoter suitable for use in various expression systems are well known in the art. In some embodiments, the promoter is a hybrid human cytomegalovirus (CMV)/TetO2 promoter. In some embodiments, the promoter is P_{AOX1}. In some embodiments, the promoter is the GAL1 inducible promoter. In some embodiments, the promoter is GPD (TDH3). In some embodiments, the promoter is the MET25 promoter. In some embodiments, the MET25 promoter is an inducible promoter.

In some embodiments, the nucleotide sequence encoding the MCP comprises a nucleotide sequence 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 4. In some embodiments, the nucleotide sequence encoding the RNA stem-loop comprises a nucleotide sequence 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In some embodiments, the nucleotide sequence encoding the MCP or RNA binding protein is codon optimized for expression in a mammalian cell. In some embodiments, the nucleotide sequence encoding the MCP or RNA binding protein is codon optimized for expression in any host cell of choice, including but not limited to, yeast cells, mammalian cells, insect cells, and bacterial cells.

In some embodiments, the mRNA display cassette further comprises a nucleotide sequence encoding a protein of interest. In some embodiments, the nucleotide sequence encoding the protein of interest comprises one or more deletions, insertions, or substitutions as compared to its wild-type sequence. In some embodiments, the mutation is a synonymous substitution. In some embodiments, the mutation is a non-synonymous substitution. In some embodiments, the protein of interest encoded by the nucleotide sequence comprises one or more deletions, insertions, or substitutions as compared to its wild-type sequence. In some embodiments, the protein of interest encoded by the nucleotide sequence comprises at least one point mutation. In some embodiments, the protein of interest encoded by the nucleotide sequence comprising one or more deletions, insertions, or substitutions has an altered function as compared to the protein of interest with the one or more deletions, insertions, or substitutions. In some embodiments, the one or more one or more deletions, insertions, or substitutions is generated using random mutagenesis techniques known in the art, for example, but not limited to error-prone PCR. In some embodiments, the one or more deletions, insertions, or substitutions is generated using rational synthesis techniques known in the art. In some embodiments, the protein of interest comprises a peptide. In some embodiments, the protein of interest comprises an artificial or in silico designed peptide. In some embodiments, the peptide has been designed for or predicted to have a specific function, such as targeting a protein or functioning as a drug. In some embodiments, the invention is directed to a protein variant library encoded by a plurality of the nucleotide sequences described herein. In some embodiments, the protein variant library comprises a plurality of in silico designed ORFs. In some embodiments, the protein variant library comprises a plurality of in silico designed peptides. In some embodiments, the protein variant library comprises a plurality of rationally designed ORFs. In some embodiments, the protein variant library comprises a plurality of rationally designed peptides. In some embodiments, the invention is directed to an in vivo mRNA display library comprising a population of variants of a single protein or a peptide library. See e.g., Example 3, Example 5. In some embodiments, the protein variant library is a peptide library designed to target a specific molecule, protein, nucleic acid or interact with a drug or chemical.

In some embodiments, the nucleic acid further comprised a nucleotide sequence comprising a universal primer sequence. In some embodiments, the nucleic acid further comprises a universal primer sequence located 5′ to the nucleotide sequence encoding the protein of interest and a universal primer sequence located 3′ to the nucleotide sequence encoding the protein of interest.

In certain aspects, the invention provides an embodiment in which a specific in vivo displayed protein is attached to an identifying sequence other than the ORF encoding the protein itself. In this embodiment of the technology, individual cells concurrently express: 1) a single protein (from the library) fused to a RNA-binding domain (e.g. capsid stem-loop recognition domain) and 2) a hybrid mRNA molecule containing both a unique molecular identifier (UMI) sequence (e.g. bar-code) and the RNA stem-loop that is recognized by the RNA-binding domain.

Unique molecular identifiers (UMIs), also referred to molecular barcodes, are known in the art. UMIs can be short sequences used to uniquely tag a molecule of interest in a sample library.

In certain aspects, the invention provides a nucleic acid comprising (i) a first cassette comprising a cloning site for insertion of a nucleotide sequence encoding a protein of interest operably linked to a nucleotide sequence encoding a MS2 bacteriophage coat protein (MCP) and (ii) a second cassette comprising a nucleotide sequence encoding a unique molecular identifier (UMI) sequence operably linked to a nucleotide sequence encoding an RNA stem-loop, wherein the MCP binds to the RNA stem-loop with high-affinity.

The invention is not limited to MCP and its cognate RNA sequence. Accordingly, in certain aspects, the invention provides a nucleic acid comprising (i) a first cassette comprising a cloning site for insertion of a nucleotide sequence encoding a protein of interest operably linked to a nucleotide sequence encoding a RNA binding protein and (ii) a second cassette comprising a nucleotide sequence encoding a unique molecular identifier (UMI) sequence operably linked to a nucleotide sequence encoding a cognate RNA sequence, wherein the RNA binding protein binds to the cognate RNA sequence with high-affinity.

In some embodiments, the nucleotide sequence encoding the MCP or RNA binding protein is located 5′ to the cloning site for insertion of the nucleotide sequence encoding the protein of interest. In some embodiments, the nucleotide sequence encoding the MCP or RNA binding protein is located 3′ to the cloning site for insertion of the nucleotide sequence encoding the protein of interest. In some embodiments, the nucleotide sequence encoding the RNA stem-loop or cognate RNA sequence is located 3′ to the nucleotide sequence encoding the UMI. In some embodiments, the nucleotide sequence encoding the RNA stem-loop or cognate RNA sequence is located in a 3′ UTR. In some embodiments, the nucleotide sequence encoding the RNA stem-loop or cognate RNA sequence is located 5′ to the nucleotide sequence encoding the UMI.

In some embodiments, a first nucleic acid comprises the first cassette and a second nucleic acid comprises the second cassette. In some embodiments, the first cassette and the second cassette are on the same nucleic acid.

In some embodiments, the nucleic acid is configured so that upon insertion of a nucleotide sequence encoding a protein of interest, the nucleotide sequence encoding the protein of interest and the MCP or RNA binding protein are operably linked so that they encode a fusion protein of the protein of interest and the MCP or RNA binding protein. In some embodiments, the fusion protein comprises the MCP or RNA binding protein fused to the N-terminus of the protein of interest. In some embodiments, the fusion protein comprises the MCP or RNA binding protein is fused to the C-terminus of the protein of interest.

In some embodiments, the nucleic acid further comprises a nucleotide sequence encoding a purification tag operably linked to the cloning site for insertion of the nucleic acid sequence encoding the protein of interest. In some embodiments, the nucleic acid is configured so that upon insertion of a nucleotide sequence encoding a protein of interest, the nucleotide sequence encoding the protein of interest and the purification tag are operably linked so that they encode a fusion protein of the protein of interest and the purification tag. In some embodiments, the fusion protein comprises the purification tag fused to the C-terminus of the protein of interest. Purification tags are well known in the art.

In some embodiments, the nucleic acid further comprises a promoter operably linked to the first cassette. In some embodiments, the nucleic acid further comprises a promoter operably linked to the second cassette. In some embodiments, the promoter is an inducible promoter. Promoter suitable for use in various expression systems are well known in the art. In some embodiments, the promoter is a hybrid human cytomegalovirus (CMV)/TetO2 promoter. In some embodiments, the promoter is P_{AOX1}. In some embodiments, the promoter is the GAL1 inducible promoter. In some embodiments, the promoter is GPD (TDH3). In some embodiments, the promoter is the MET25 promoter. In some embodiments, the MET25 promoter is an inducible promoter.

In some embodiments, the first cassette and second cassette are encoded by a single nucleic acid capable of producing a first RNA molecule and a second RNA molecule. In some embodiments, the first RNA molecule is a mRNA molecule encoding the protein of interest and the MCP or RNA binding protein. In some embodiments, the second RNA molecule encodes the UMI sequence and a RNA stem-loop or cognate RNA sequence. In some embodiments, the MCP or RNA binding protein encoded by the first RNA molecule binds with high affinity to the RNA stem-loop or cognate RNA sequence encoded by the second RNA molecule.

In some embodiments, the UMI uniquely identifies the protein of interest.

In some embodiments, the nucleotide sequence encoding the MCP comprises a nucleotide sequence 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 4. In some embodiments, the nucleotide sequence encoding the RNA stem-loop comprises a nucleotide sequence 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In some embodiments, the nucleotide sequence encoding the MCP or RNA binding protein is codon optimized for expression in a mammalian cell. In some embodiments, the nucleotide sequence encoding the MCP or RNA binding protein is codon optimized for expression in any host cell of choice, including but not limited to, yeast cells, mammalian cells, human cells, insect cells, and bacterial cells. In some embodiments, the technology described herein can be used for proteomic analysis in bacterial cells, insect cells, human cell lines, mammalian cell lines, or animal models.

In some embodiments, the mRNA display cassette further comprises a nucleotide sequence encoding a protein of interest. In some embodiments, the nucleotide sequence encoding the protein of interest comprises one or more deletions, insertions, or substitutions as compared to its wild-type sequence. In some embodiments, the mutation is a synonymous substitution. In some embodiments, the mutation is a non-synonymous substitution. In some embodiments, the protein of interest encoded by the nucleotide sequence comprises one or more deletions, insertions, or substitutions as compared to its wild-type sequence. In some embodiments, the protein of interest encoded by the nucleotide sequence comprises at least one point mutation. In some embodiments, the protein of interest encoded by the nucleotide sequence comprising one or more deletions, insertions, or substitutions has an altered function as compared to the protein of interest with the one or more deletions, insertions, or substitutions. In some embodiments, the one or more one or more deletions, insertions, or substitutions is generated using random mutagenesis techniques known in the art, for example, but not limited to error-prone PCR. In some embodiments, the one or more deletions, insertions, or substitutions is generated using rational synthesis techniques known in the art. In some embodiments, the protein of interest comprises a peptide. In some embodiments, the protein of interest comprises an artificial or in silico designed peptide. In some embodiments, the peptide has been designed for or predicted to have a specific function, such as targeting a protein or functioning as a drug. In some embodiments, the invention is directed to a protein variant library encoded by a plurality of the nucleotide sequences described herein. In some embodiments, the protein variant library comprises a plurality of in silico designed ORFs. In some embodiments, the protein variant library comprises a plurality of in silico designed peptides. In some embodiments, the protein variant library comprises a plurality of rationally designed ORFs. In some embodiments, the protein variant library comprises a plurality of rationally designed peptides. In some embodiments, the invention is directed to an in vivo mRNA display library comprising a population of variants of a single protein or a peptide library. See e.g., Example 3, Example 5. In some embodiments, the protein variant library is a peptide library designed to target a specific molecule, protein, nucleic acid or interact with a drug or chemical.

In some embodiments, the first cassette and/or second cassette of the nucleic acid further comprise a nucleotide sequence comprising a universal primer sequence. In some embodiments, the nucleic acid further comprises a universal primer sequence located 5′ to the nucleotide sequence encoding the protein of interest and a universal primer sequence located 3′ to the nucleotide sequence encoding the protein of interest. In some embodiments, the nucleic acid further comprises a universal primer sequence located 5′ to the nucleotide sequence encoding the second cassette and a universal primer sequence located 3′ to the second cassette.

In some embodiments, the second cassette further comprises a terminator sequence operatively linked to the nucleotide sequence encoding the UMI and RNA stem-loop or cognate RNA sequence. In some embodiments, a UMI is a random sequence of nucleotides. In some embodiments, the UMI is a random sequence of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some embodiments, the UMI is a random sequence of 20 to 30 nucleotides. In some embodiments, the UMI is long enough to provide a unique sequence for all ORFs in an in vivo mRNA display library. In some embodiments, the second cassette is 50 to 100 nucleotides long. In some embodiments, each protein in the in vivo mRNA display library described herein is identified by a UMI.

In some embodiments, the nucleic acid of the invention is configured for expression in a yeast cell. In some embodiments, the yeast cell is a Pichia cell. In some embodiments, the host cell is a Hansenula cell. In some embodiments, the yeast cell is a Schizosaccharomyces cell. In some embodiments, the yeast cell is a Kluyveromyces cell. In some embodiments, the yeast cell is a Yarrowia cell. In some embodiments, the yeast cell is a Debaryomyces cell. In some embodiments, the yeast cell is a Candida cell. In some embodiments, the yeast cell is Saccharomyces cerevisiae. In some embodiments, the protein of interest is a yeast protein. In some embodiments, the yeast protein is a Saccharomyces cerevisiae protein. In some embodiments, the technology described herein can be used for proteomic analysis in yeast cells.

In some embodiments, the nucleic acid of the invention is configured for expression in a mammalian cell. Mammalian expression systems are known in the art. In some embodiments, the mammalian cell is a Chinese hamster ovary (CHO) cell, a baby hamster kidney (BHK) cell, a mouse myeloma (NS0 or SP2/0) cell, a rat myeloma (YB2/0) cell. In some embodiments, the mammalian cell is a human cell. In some embodiments, the human cell is a HEK cell. In some embodiments, the human cell is a HT-1080 cell. In some embodiments, the human cell is a PER.C6 cell. In some embodiments the human cell is a Huh-7 cell. In some embodiments, the human cell is a HeLa cell. In some embodiments, the protein of interest is a mammalian protein. In some embodiments, the mammalian protein is a human protein. In some embodiments, the promoter is a hybrid human cytomegalovirus (CMV)/TetO2 promoter. In some embodiments, the technology described herein can be used for proteomic analysis in human cells, mammalian cells, or animal models.

In some embodiments, the nucleic acid of the invention is configured for expression in an insect cell. Insect expression systems are known in the art. In some embodiments, the insect cell is derived from derived from Bombyx mori, Mamestra brassicae, Spodoptera frugiperda, Trichoplusia ni, and Drosophila melanogaster. In some embodiments, the insect cell is Sf9 cell line. In some embodiments, the protein of interest is a mammalian protein. In some embodiments, the mammalian protein is a human protein. In some embodiments, the technology described herein can be used for proteomic analysis in insect cells.

In some embodiments, the nucleic acid of the invention is configured for expression in a bacterial cell. Bacterial expression systems are known in the art. In some embodiments, the protein of interest is a bacterial protein. In some embodiments, the technology described herein can be used for proteomic analysis in bacterial cells.

### Population of Nucleic Acids of the Invention

In certain aspects, the invention provides a population of the nucleic acids of the invention described herein.

In certain aspects, the invention provides a population of nucleic acids, each nucleic acid of the population comprising a mRNA display cassette, the mRNA display cassette comprising a nucleotide sequence encoding a protein of interest operably linked to (i) a nucleotide sequence encoding a MS2 bacteriophage coat protein (MCP) and (ii) to a nucleotide sequence encoding an RNA stem-loop, wherein the MCP binds to the RNA stem-loop with high-affinity.

The invention is not limited to MCP and its cognate RNA sequence. Accordingly, in certain aspects, the invention provides a population of nucleic acids, each nucleic acid of the population comprising a mRNA display cassette, the mRNA display cassette comprising a nucleotide sequence encoding a protein of interest operably linked to (i) a nucleotide sequence encoding a RNA binding protein and (ii) to a nucleotide sequence encoding an cognate RNA sequence, wherein the RNA binding protein binds to the cognate RNA sequence with high-affinity.

In some embodiments, for each nucleic acid of the population the nucleotide sequence encoding the MCP or RNA binding protein is located 5′ to the nucleotide sequence encoding the protein of interest. In some embodiments, for each nucleic acid of the population the nucleotide sequence encoding the MCP or RNA binding protein is located 3′ to the cloning site for insertion of the nucleotide sequence encoding the protein of interest. In some embodiments, for each nucleic acid of the population the nucleotide sequence encoding the RNA stem-loop is located 3′ to the nucleotide sequence encoding the protein of interest. In some embodiments, for each nucleic acid of the population the nucleotide sequence encoding the RNA stem-loop or cognate RNA sequence is located in a 3′ UTR. In some embodiments, for each nucleic acid in the population the nucleotide sequence encoding the RNA stem-loop or cognate RNA sequence is located 5′ to the nucleotide sequence encoding the MCP or RNA binding protein.

In some embodiments, for each nucleic acid of the population the nucleotide sequence encoding the protein of interest and the MCP or RNA binding protein are operably linked so that they encode a fusion protein of the protein of interest and the MCP or RNA binding protein. In some embodiments, for each nucleic acid of the population the fusion protein comprises the MCP or RNA binding protein fused to the N-terminus of the protein of interest. In some embodiments, for each nucleic acid of the population the fusion protein comprises the MCP or RNA binding protein is fused to the C-terminus of the protein of interest.

In some embodiments, for each nucleic acid of the population the mRNA display cassette further comprises a nucleotide sequence encoding a purification tag operably linked to the nucleic acid sequence encoding the protein of interest. In some embodiments, for each nucleic acid of the population the nucleotide sequence encoding the protein of interest and the purification tag are operably linked so that they encode a fusion protein of the protein of interest and the purification tag. In some embodiments, for each nucleic acid of the population the fusion protein comprises the purification tag fused to the C-terminus of the protein of interest. Purification tags are well known in the art.

In some embodiments, each nucleic acid of the population further comprises a promoter operably linked to the mRNA display cassette. In some embodiments, the promoter is an inducible promoter. Promoter suitable for use in various expression systems are well known in the art. In some embodiments, the promoter is a hybrid human cytomegalovirus (CMV)/TetO2 promoter. In some embodiments, the promoter is P_{AOX1}. In some embodiments, the promoter is the GAL1 inducible promoter. In some embodiments, the promoter is GPD (TDH3). In some embodiments, the promoter is the MET25 promoter. In some embodiments, the MET25 promoter is an inducible promoter.

In some embodiments, for each nucleic acid of the population the nucleotide sequence encoding the MCP comprises a nucleotide sequence 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 4. In some embodiments, for each nucleic acid of the population the nucleotide sequence encoding the RNA stem-loop comprises a nucleotide sequence 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In some embodiments, the nucleotide sequence encoding the MCP or RNA binding protein is codon optimized for expression in a mammalian cell. In some embodiments, the nucleotide sequence encoding the MCP or RNA binding protein is codon optimized for expression in any host cell of choice, including but not limited to, yeast cells, mammalian cells, insect cells, and bacterial cells.

In some embodiments, for each nucleic acid of the population the mRNA expression cassette further comprises a nucleotide sequence encoding a protein of interest. In some embodiments, at least one of the nucleic acids of the population comprises a nucleotide sequence encoding a protein of interest, wherein the nucleotide sequence comprises one or more deletions, insertions, or substitutions as compared to its wild-type sequence. In some embodiments, the mutation is a synonymous substitution. In some embodiments, the mutation is a non-synonymous substitution. In some embodiments, at least one of the nucleic acids of the population comprises a nucleotide sequence encoding a protein of interest, wherein the protein of interest encoded by the nucleotide sequence comprises one or more deletions, insertions, or substitutions as compared to its wild-type sequence. In some embodiments, the protein of interest encoded by the nucleotide sequence comprises at least one point mutation. In some embodiments, the one or more one or more deletions, insertions, or substitutions is generated using random mutagenesis techniques known in the art, for example, but not limited to error-prone PCR. In some embodiments, the one or more deletions, insertions, or substitutions is generated using rational synthesis techniques known in the art. In some embodiments, the protein of interest comprises a peptide. In some embodiments, the protein of interest comprises an artificial or in silico designed peptide. In some embodiments, the peptide has been designed for or predicted to have a specific function, such as targeting a protein or functioning as a drug. In some embodiments, the population of nucleic acids encodes variant sequences of one or more proteins of interest (e.g., a protein variant library). In some embodiments, the population of nucleic acids encodes variant sequences of a single protein of interest. In some embodiments, the invention is directed to an in vivo mRNA display library comprising a population of variants of a single protein or a peptide library. See e.g., Example 3, Example 5. In some embodiments, the variant sequences of one or more proteins of interest comprises a plurality of in silico designed ORFs. In some embodiments, variant sequences of one or more proteins of interest comprises a plurality of in silico designed peptides. In some embodiments, the variant sequences of one or more proteins of interest comprises a plurality of rationally designed ORFs. In some embodiments, variant sequences of one or more proteins of interest comprises a plurality of rationally designed peptides. In some embodiments, the protein variant library is a peptide library designed to target a specific molecule, protein, nucleic acid or interact with a drug or chemical. In some embodiments, the population of nucleic acids is a mutagenized library comprising a population of mutagenized proteins of interest. In some embodiments, the population of mutagenized proteins have altered functions as compared to its non-mutagenized variant. In some embodiments, the protein function is related to protein expression, folding, stability, enzymatic activity, signaling, regulation, sub-cellular localization, or interactions with at least one target.

In some embodiments, each nucleic acid of the population further comprises a nucleotide sequence comprising a universal primer sequence. In some embodiments, each nucleic acid of the population further comprises a universal primer sequence located 5′ to the nucleotide sequence encoding the protein of interest and a universal primer sequence located 3′ to the nucleotide sequence encoding the protein of interest.

In some embodiments, each nucleic acid of the population comprises a nucleotide sequence encoding a different protein of interest. In some embodiments, the nucleic acids of the population comprise nucleotide sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest. In some embodiments, the nucleic acids of the population comprises nucleotide sequences encoding different proteins of interest that are representative of a proteome of interest. In some embodiments, the proteome of interest is the proteome of Saccharomyces cerevisiae. In some embodiments, the proteome of interest is the proteome of a mammalian cell. In some embodiments, the proteome of interest is the proteome of a human cell.

In certain aspects the invention provides, a population of nucleic acids, each nucleic acid of the population comprising (i) a first cassette comprising a cloning site for insertion of a nucleotide sequence encoding a protein of interest operably linked to a nucleotide sequence encoding a MS2 bacteriophage coat protein (MCP) and (ii) a second cassette comprising a nucleotide sequence encoding a unique molecular identifier (UMI) sequence operably linked to a nucleotide sequence encoding an RNA stem-loop, wherein the MCP binds to the RNA stem-loop with high-affinity.

The invention is not limited to MCP and its cognate RNA sequence. Accordingly, in certain aspects the invention provides, a population of nucleic acids, each nucleic acid of the population comprising (i) a first cassette comprising a cloning site for insertion of a nucleotide sequence encoding a protein of interest operably linked to a nucleotide sequence encoding a RNA binding protein and (ii) a second cassette comprising a nucleotide sequence encoding a unique molecular identifier (UMI) sequence operably linked to a nucleotide sequence encoding a cognate RNA sequence, wherein the RNA binding protein binds to the cognate RNA sequence with high-affinity.

In some embodiments, for each nucleic acid of the population the nucleotide sequence encoding the MCP or RNA binding protein is located 5′ to the nucleotide sequence encoding the protein of interest. In some embodiments, for each nucleic acid of the population the nucleotide sequence encoding the MCP or RNA binding protein is located 3′ to the nucleotide sequence encoding the protein of interest. In some embodiments, for each nucleic acid of the population the nucleotide sequence encoding the RNA stem-loop or cognate RNA sequence is located 3′ to the nucleotide sequence encoding the UMI. In some embodiments, for each nucleic acid of the population the nucleotide sequence encoding the RNA stem-loop is located in a 3′ UTR. In some embodiments, for each nucleic acid of the population the nucleotide sequence encoding the RNA stem-loop or cognate RNA sequence is located 5′ to the nucleotide sequence encoding the UMI.

In some embodiments, a first nucleic acid comprises the first cassette and a second nucleic acid comprises the second cassette. In some embodiments, the first cassette and the second cassette are on the same nucleic acid.

In some embodiments, for each nucleic acid of the population the nucleotide sequence encoding the protein of interest and the MCP or RNA binding protein are operably linked so that they encode a fusion protein of the protein of interest and the MCP or RNA binding protein. In some embodiments, for each nucleic acid of the population the fusion protein comprises the MCP or RNA binding protein fused to the N-terminus of the protein of interest. In some embodiments, for each nucleic acid of the population the fusion protein comprises the MCP or RNA binding protein fused to the C-terminus of the protein of interest.

In some embodiments, for each nucleic acid of the population the mRNA display cassette further comprises a nucleotide sequence encoding a purification tag operably linked to the nucleic acid sequence encoding the protein of interest. In some embodiments, for each nucleic acid of the population the nucleotide sequence encoding the protein of interest and the purification tag are operably linked so that they encode a fusion protein of the protein of interest and the purification tag. In some embodiments, for each nucleic acid of the population the fusion protein comprises the purification tag fused to the C-terminus of the protein of interest. Purification tags are well known in the art.

In some embodiments, each nucleic acid of the population further comprises a promoter operably linked to the mRNA display cassette. In some embodiments, the promoter is an inducible promoter. Promoter suitable for use in various expression systems are well known in the art. In some embodiments, the promoter is a hybrid human cytomegalovirus (CMV)/TetO2 promoter. In some embodiments, the promoter is P_{AOX1}. In some embodiments, the promoter is the GAL1 inducible promoter. In some embodiments, the promoter is GPD (TDH3).

In some embodiments, each of the first cassette and second cassette are encoded by a single nucleic acid capable of producing a first RNA molecule and a second RNA molecule. In some embodiments, the first RNA molecule is a mRNA molecule encoding the protein of interest and the MCP or RNA binding protein. In some embodiments, the second RNA molecule encodes the UMI sequence and a RNA stem-loop or cognate RNA sequence. In some embodiments, the MCP or RNA binding protein encoded by the first RNA molecule binds with high affinity to the RNA stem-loop or cognate RNA sequence encoded by the second RNA molecule.

In some embodiments, the UMI uniquely identifies the protein of interest.

In some embodiments, for each nucleic acid of the population the nucleotide sequence encoding the MCP comprises a nucleotide sequence 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 4. In some embodiments, for each nucleic acid of the population the nucleotide sequence encoding the RNA stem-loop comprises a nucleotide sequence 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In some embodiments, the nucleotide sequence encoding the MCP or RNA binding protein is codon optimized for expression in a mammalian cell. In some embodiments, the nucleotide sequence encoding the MCP or RNA binding protein is codon optimized for expression in any host cell of choice, including but not limited to, yeast cells, mammalian cells, insect cells, and bacterial cells.

In some embodiments, for each nucleic acid of the population the mRNA expression cassette further comprises a nucleotide sequence encoding a protein of interest. In some embodiments, at least one of the nucleic acids of the population comprises a nucleotide sequence encoding a protein of interest, wherein the nucleotide sequence comprises one or more deletions, insertions, or substitutions as compared to its wild-type sequence. In some embodiments, the mutation is a synonymous substitution. In some embodiments, the mutation is a non-synonymous substitution. In some embodiments, at least one of the nucleic acids of the population comprises a nucleotide sequence encoding a protein of interest, wherein the protein of interest encoded by the nucleotide sequence comprises one or more deletions, insertions, or substitutions as compared to its wild-type sequence. In some embodiments, the protein of interest encoded by the nucleotide sequence comprises at least one point mutation. In some embodiments, the one or more one or more deletions, insertions, or substitutions is generated using random mutagenesis techniques known in the art, for example, but not limited to error-prone PCR. In some embodiments, the one or more deletions, insertions, or substitutions is generated using rational synthesis techniques known in the art. In some embodiments, the protein of interest comprises a peptide. In some embodiments, the protein of interest comprises an artificial or in silico designed peptide. In some embodiments, the peptide has been designed for or predicted to have a specific function, such as targeting a protein or functioning as a drug. In some embodiments, the population of nucleic acids encodes variant sequences of one or more proteins of interest (e.g., a protein variant library). In some embodiments, the population of nucleic acids encodes variant sequences of a single protein of interest. In some embodiments, the invention is directed to an in vivo mRNA display library comprising a population of variants of a single protein or a peptide library. See e.g., Example 3, Example 5. In some embodiments, the variant sequences of one or more proteins of interest comprises a plurality of in silico designed ORFs. In some embodiments, variant sequences of one or more proteins of interest comprises a plurality of in silico designed peptides. In some embodiments, the variant sequences of one or more proteins of interest comprises a plurality of rationally designed ORFs. In some embodiments, variant sequences of one or more proteins of interest comprises a plurality of rationally designed peptides. In some embodiments, the protein variant library is a peptide library designed to target a specific molecule, protein, nucleic acid or interact with a drug or chemical. In some embodiments, the population of nucleic acids is a mutagenized library comprising a population of mutagenized proteins of interest. In some embodiments, the population of mutagenized proteins have altered functions as compared to its non-mutagenized variant. In some embodiments, the protein function is related to protein expression, folding, stability, enzymatic activity, signaling, regulation, sub-cellular localization, or interactions with at least one target.

In some embodiments, each first cassette and/or second cassette of the nucleic acid of the population further comprises a nucleotide sequence comprising a universal primer sequence. In some embodiments, each nucleic acid of the population further comprises a universal primer sequence located 5′ to the nucleotide sequence encoding the protein of interest and a universal primer sequence located 3′ to the nucleotide sequence encoding the protein of interest. In some embodiments, each nucleic acid of the population further comprises a universal primer sequence located 5′ to the nucleotide sequence encoding the second cassette and a universal primer sequence located 3′ to the second cassette.

In some embodiments, each nucleic acid of the population comprises a nucleotide sequence encoding a different protein of interest. In some embodiments, the nucleic acids of the population comprise nucleotide sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest. In some embodiments, the nucleic acids of the population comprises nucleotide sequences encoding different proteins of interest that are representative of a proteome of interest. In some embodiments, the proteome of interest is the proteome of Saccharomyces cerevisiae. In some embodiments, the proteome of interest is the proteome of a mammalian cell. In some embodiments, the proteome of interest is the proteome of a human cell. In some embodiments, the proteome of interest is the proteome of bacterial cells, insect cells, human cell lines, mammalian cell lines, or animal models.

In some embodiments, each of the second cassettes further comprises a terminator sequence operatively linked to the nucleotide sequence encoding the UMI and RNA stem-loop or cognate RNA sequence. In some embodiments, a UMI is a random sequence of nucleotides. In some embodiments, the UMI is a random sequence of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some embodiments, the UMI is a random sequence of 20 to 30 nucleotides. In some embodiments, the UMI is long enough to provide a unique sequence for all ORFs in an in vivo mRNA display library. In some embodiments, the second cassette is 50 to 100 nucleotides long. In some embodiments, each protein in the in vivo mRNA display library described herein is identified by a UMI.

In some embodiments, each nucleic acid of the population of nucleic acids is in a vector.

In some embodiments, the nucleic acids of the population of nucleic acids of the invention is configured for expression in a yeast cell. In some embodiments, the yeast cell is a Pichia cell. In some embodiments, the host cell is a Hansenula cell. In some embodiments, the yeast cell is a Schizosaccharomyces cell. In some embodiments, the yeast cell is a Kluyveromyces cell. In some embodiments, the yeast cell is a Yarrowia cell. In some embodiments, the yeast cell is a Debaryomyces cell. In some embodiments, the yeast cell is a Candida cell. In some embodiments, the yeast cell is Saccharomyces cerevisiae. In some embodiments, the protein of interest encoded by each of the nucleic acids of the population is a yeast protein. In some embodiments, the yeast protein is a Saccharomyces cerevisiae protein.

In some embodiments, the nucleic acid of the population of nucleic acids of the invention is configured for expression in a mammalian cell. Mammalian expression systems are known in the art. In some embodiments, the mammalian cell is a Chinese hamster ovary (CHO) cell, a baby hamster kidney (BHK) cell, a mouse myeloma (NS0 or SP2/0) cell, a rat myeloma (YB2/0) cell. In some embodiments, the mammalian cell is a human cell. In some embodiments, the human cell is a HEK cell. In some embodiments, the human cell is a HT-1080 cell. In some embodiments, the human cell is a PER.C6 cell. In some embodiments the human cell is a Huh-7 cell. In some embodiments, the human cell is a HeLa cell. In some embodiments, the protein of interest encoded by each of the nucleic acids of the population is a mammalian protein. In some embodiments, the mammalian protein is a human protein. In some embodiments, the promoter is a hybrid human cytomegalovirus (CMV)/TetO2 promoter.

In some embodiments, the nucleic acid of the population of nucleic acids of the invention is configured for expression in an insect cell. Insect expression systems are known in the art. In some embodiments, the insect cell is derived from derived from Bombyx mori, Mamestra brassicae, Spodoptera frugiperda, Trichoplusia ni, and Drosophila melanogaster. In some embodiments, the insect cell is Sf9 cell line. In some embodiments, the protein of interest encoded by each of the nucleic acids of the population is a mammalian protein. In some embodiments, the mammalian protein is a human protein.

In some embodiments, the nucleic acid of the population of nucleic acids of the invention is configured for expression in a bacterial cell. Bacterial expression systems are known in the art. In some embodiments, the protein of interest encoded by each of the nucleic acids of the population is a bacterial protein.

### Vectors of the Invention

In certain aspects, the invention provides a vector comprising any one of the nucleic acids described herein. Methods of cloning nucleic acids into vectors is well known in the art.

In some embodiments, the vector is a transient expression vector. In some embodiments, the vector is a stable expression vector. In some embodiments the vector is a genomically integrating vector. In some embodiments, the vector is a yeast vector. In some embodiments, the vector is a mammalian vector. In some embodiments, the vector is an insect cell vector. In some embodiments, the vector is a bacterial vector.

In some embodiments, the vector comprises a pcDNA™ FRT/TO vector. The pcDNA™ FRT/TO vector is a 5.1 kb inducible expression vector for use with the Flp-In™ T-REx™ System. A detailed description of this vector can be found on the ThermoFischer Scientific website for pcDNA™ 5/FRT/TO Vector Kit (catalog number: V652020), the contents of which is hereby incorporated by reference in its entirety.

### Host Cells of the Invention

In certain aspects, the invention provides a host cell comprising a vector as described herein. In certain aspects, the invention provides a population of host cells, wherein each host cell comprises a vector from the population of vectors as described herein. Methods for introducing vectors into host cells are known in the art.

In some embodiments, the host cell is a mammalian cell. In some embodiments, the mammalian cell is an immortalized Chinese hamster ovary (CHO) cell. In some embodiments, the mammalian cell is a baby hamster kidney (BHK) cell. In some embodiments, the mammalian cell is a mouse myeloma (NS0 or SP2/0) cell. In some embodiments, the mammalian cell is a rat myeloma (YB2/0) cell. In some embodiments, the host cell is a human cell. In some embodiments, the human cell is a HEK cell. In some embodiments, the human cell is a HT-1080 cell. In some embodiments, the human cell is a PER.C6 cell. In some embodiments the human cell is a Huh-7 cell. In some embodiments, the human cell is a HeLa cell.

In some embodiments, the host cell is yeast cell. In some embodiments, the host cell is a Saccharomyces cell. In some embodiments, the yeast cell is a Pichia cell. In some embodiments, the yeast cell is a Hansenula cell. In some embodiments, the yeast cell is a Schizosaccharomyces cell. In some embodiments, the yeast cell is a Kluyveromyces cell. In some embodiments, the host cell is a Yarrowia cell. In some embodiments, the yeast cell is a Debaryomyces cell. In some embodiments, the yeast cell is a Candida cell. In some embodiments, the host cell is a Saccharomyces cerevisiae cell.

Method of Producing a Population of Cells Comprising an In Vivo mRNA Display Library

In certain aspects, the invention provides a method of producing a population of cells comprising an in vivo mRNA display library, the method comprising: (a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; (b) allowing the expression of the said one or more nucleic acid sequences; (c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest.

In certain aspects, the invention provides a method of producing a population of cells comprising an in vivo mRNA display library, the method comprising: a) providing a population of cells, wherein each cell comprises one or more nucleic acid sequences encoding: a RNA-binding protein; a protein of interest; a unique molecular identifier (UMI); and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the unique molecular identifier comprises the cognate RNA sequence; b) allowing the expression of the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the unique molecular identifier.

In some embodiments, the RNA-binding protein is an RNA-binding capsid protein and optionally, wherein the cognate RNA-binding sequence is an RNA stem-loop sequence. In some embodiments, the RNA-binding capsid protein is MS2 bacteriophage coat protein (MCP).

In some embodiments, the nucleic acid sequence encoding the RNA-binding protein is located 5′ to the nucleic acid sequence encoding the protein of interest. In some embodiments, the nucleotide sequence encoding the RNA binding protein is located 3′ to the cloning site for insertion of the nucleotide sequence encoding the protein of interest. In some embodiments, the nucleic acid sequence encoding the cognate RNA sequence is located 3′ to the nucleic acid sequence encoding the protein of interest. In some embodiments, the nucleic acid sequence encoding the cognate RNA sequence is located 3′ to the nucleic acid sequence encoding the UMI. In some embodiments, the nucleotide sequence encoding the RNA stem-loop or cognate RNA sequence is located in a 3′ UTR. In some embodiments, the nucleic acid sequence encoding the cognate RNA sequence is located in a 3′ UTR of the mRNA sequence encoding the protein of interest. In some embodiments, the nucleic acid sequence encoding the cognate RNA sequence is located 5′ to the nucleic acid sequence encoding the protein of interest. In some embodiments, the nucleic acid sequence encoding the cognate RNA sequence is located 5′ to the nucleic acid sequence encoding the UMI. In some embodiments, the fusion protein comprises the RNA-binding protein fused to the N-terminus of the protein of interest. In some embodiments, the fusion protein comprises the MCP or RNA binding protein is fused to the C-terminus of the protein of interest.

In some embodiments, the one or more nucleic acid sequences further encodes a purification tag wherein the nucleic acid sequence encoding the protein of interest and the purification tag are operably linked so that they encode a fusion protein of the protein of interest and the purification tag. In some embodiments, the fusion protein comprises the purification tag fused to the C-terminus of the protein of interest. Purification tags are well known in the art. In some embodiments, the purification tag is a FLAG tag, a MYC tag, a HIS tag, or a green fluorescent protein (GFP).

In some embodiments, the one or more nucleic acid sequences further comprise a nucleic acid sequence comprising a universal primer sequence. In some embodiments, a universal primer sequence is located 5′ to the nucleic acid sequence encoding the protein of interest and a universal primer sequence is located 3′ to the nucleic acid sequence encoding the protein of interest. In some embodiments, a universal primer sequence is located 5′ to the nucleic acid sequence encoding the UMI and a universal primer sequence is located 3′ to the nucleic acid sequence encoding the UMI.

In some embodiments, the nucleic acid sequence encoding the RNA-binding capsid protein comprises a nucleic acid sequence 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 4. In some embodiments, the nucleic acid sequence encoding the RNA stem-loop comprises a nucleic acid sequence 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In some embodiments, the nucleotide sequence encoding the MCP or RNA binding protein is codon optimized for expression in a mammalian cell. In some embodiments, the nucleotide sequence encoding the MCP or RNA binding protein is codon optimized for expression in any host cell of choice, including but not limited to, yeast cells, mammalian cells, insect cells, and bacterial cells.

In some embodiments, each cell in the population of cells comprises a nucleic acid sequence encoding the same protein of interest. In some embodiments, each cell in the population of cells comprises a nucleic acid sequence encoding a different protein of interest.

In some embodiments, the population of cells comprise nucleic acid sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest.

In some embodiments, the population of cells comprise nucleic acids sequences encoding different proteins of interest that are representative of a proteome of interest. In some embodiments, the proteome of interest is the proteome of the cells. In some embodiments, the cells are Saccharomyces cerevisiae. In some embodiments, the proteome of interest is the proteome of the mammalian cells. In some embodiments, the cells are human cells. In some embodiments, the technology described herein can be used for proteomic analysis in bacterial cells, insect cells, human cell lines, mammalian cell lines, or animal models.

In some embodiments, the nucleic acid sequence encoding the protein of interest comprises one or more deletions, insertions, or mutations as compared to its wild-type sequence as described herein. In some embodiment, the protein of interest encoded by the nucleic acid sequence comprises one or more deletions, insertions, or mutations as compared to its wild-type sequence as described herein.

In some embodiments, the nucleic acid sequence further comprises a terminator sequence operatively linked to the nucleotide sequence encoding the UMI and RNA stem-loop or cognate RNA sequence. In some embodiments, a UMI is a random sequence of nucleotides. In some embodiments, the UMI is a random sequence of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some embodiments, the UMI is a random sequence of 20 to 30 nucleotides. In some embodiments, the UMI is long enough to provide a unique sequence for all ORFs in an in vivo mRNA display library. In some embodiments, the second cassette is 50 to 100 nucleotides long. In some embodiments, each protein in the in vivo mRNA display library described herein is identified by a UMI.

### Method of Performing High Throughput Proteomics

In certain aspects, the invention provides a method of performing high throughput proteomics, the method comprising: (a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; wherein the population of cells comprise nucleic acid sequences encoding a plurality of different proteins of interest; (b) expressing the said one or more nucleic acid sequences; (c) incubating the cells, thereby producing a population of cells comprising an in vivo mRNA display, wherein the expressed RNA-binding protein-protein of interest fusion protein binds to the cognate RNA sequence present on the mRNA sequence encoding the protein of interest; (d) lysing the population of cells; (e) performing a biochemical assay on a portion of the lysate of step d) to generate an enriched lysate; (f) detecting the amount of mRNA encoding each protein of interest and comprising the RNA stem-loop that is present in the lysate of step d); (g) detecting the amount of mRNA encoding each protein of interest and comprising the RNA stem-loop that is present in the enriched lysate of step e); and (h) for each protein of interest, comparing the amount of mRNA detected in step f) and step g), wherein a protein of interest is determined to be enriched under the biochemical assay conditions if an increased level of mRNA detected in step g) compared to step f) or wherein a protein of interest is determined to be depleted under the biochemical assay conditions is a decreased level of mRNA is detected in step g) compared to step f).

In certain aspects, the invention provides a method of performing high throughput proteomics, the method comprising: a) providing a population of cells, wherein each cell comprises one or more nucleic acid sequences encoding: a RNA-binding protein; a protein of interest; a unique molecular identifier (UMI); and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the UMI comprises the cognate RNA sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the UMI; d) lysing the population of cells; e) performing a biochemical assay on a portion of the lysate of step d) to generate an enriched lysate; f) detecting the amount of mRNA encoding each UMI and comprising the RNA stem-loop that is present in the lysate of step d); g) detecting the amount of mRNA encoding each UMI and comprising the RNA stem-loop that is present in the enriched lysate of step e); and h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).

In some embodiments, a protein of interest is determined to be depleted under the biochemical assay conditions if there is a Log₂ fold depletion of less than −2.

In some embodiments, the biochemical assay is an immunoprecipitation with a phosphor-specific antibody. In some embodiments, an environmental or genetic perturbation is performed between step d) and e).

In some embodiments, the RNA-binding domain is an RNA-binding capsid protein and optionally, wherein the cognate RNA-binding sequence is an RNA stem-loop sequence. In some embodiments, the detecting of steps f) and g) is performed using next generation sequencing. In some embodiments, the detecting of steps f) and g) is performed by i) reverse transcribing the mRNAs encoding the proteins of interest and comprising the RNA stem-loop; ii) performing a second strand synthesis on the reverse transcription product; iii) fragmenting the second strand synthesis product; iv) ligating nucleic acid linkers to the fragmented nucleic acids; v) amplifying the ligated nucleic acids; and vi) sequencing the amplified nucleic acids.

In some embodiments, the reverse transcription uses a primer specific for a universal primer sequence located 3′ to the nucleic acid sequence encoding the protein of interest. In some embodiments, the second strand synthesis uses a primer specific for a universal primer sequence located 5′ to the nucleic acid sequence encoding the protein of interest. In some embodiments, the amplification uses a primer specific to the linker and a primer specific to a universal primer sequence located 5′ to the nucleic acid sequence encoding the protein of interest and/or a universal primer sequence located 3′ to the nucleic acid sequence encoding the protein of interest.

In some embodiments, the amplification comprises a first and a second amplification. In some embodiments, the amplification adds sequencing indexes and adaptors to the nucleic acids. In some embodiments, the sequencing is Illumina sequencing.

In some embodiments, the biochemical assay in an immunoprecipitation assay or a subcellular fractionation. In some embodiments, the biochemical assay is an assay that enriches for a DNA or RNA bait in order to identify proteins that bind to the DNA or RNA bait via the mRNA library readout. For example, a RNA or DNA of interest can be expressed in the population of cells with an affinity handle, such as, but not limited to, an aptamer. Alternatively, a RNA or DNA of interest can be added to the library lysate after lysis of the cells. The biochemical assay uses the affinity handle to generate a sample enriched for proteins that bind to the DNA or RNA molecule comprising the affinity handle.

In some embodiments, the RNA-binding protein is MCP. In some embodiments, the nucleic acid sequence encoding the RNA-binding protein is located 5′ to the nucleic acid sequence encoding the protein of interest. In some embodiments, the nucleic acid sequence encoding the RNA-binding protein is located 3′ to the nucleic acid sequence encoding the protein of interest. In some embodiments, the nucleic acid sequence encoding the cognate RNA sequence is located 3′ to the nucleic acid sequence encoding the protein of interest. In some embodiments, the nucleic acid sequence encoding the cognate RNA sequence is located 3′ to the nucleic acid sequence encoding the UMI. In some embodiments, the nucleotide sequence encoding the RNA stem-loop or cognate RNA sequence is located in a 3′ UTR. In some embodiments, the nucleic acid sequence encoding the cognate RNA sequence is located in a 3′ UTR of the mRNA sequence encoding the protein of interest. In some embodiments, the nucleic acid sequence encoding the cognate RNA sequence is located 5′ to the nucleic acid sequence encoding the protein of interest. In some embodiments, the nucleic acid sequence encoding the cognate RNA sequence is located 5′ to the nucleic acid sequence encoding the UMI. In some embodiments, the fusion protein comprises the RNA binding protein is fused to the C-terminus of the protein of interest. In some embodiments, the fusion protein comprises the RNA binding protein is fused to the N-terminus of the protein of interest.

In some embodiments, the fusion protein comprises the RNA-binding protein fused to the N-terminus of the protein of interest. In some embodiments, the one or more nucleic acid sequences further encodes a purification tag wherein the nucleic acid sequence encoding the protein of interest and the purification tag are operably linked so that they encode a fusion protein of the protein of interest and the purification tag. In some embodiments, the fusion protein comprises the purification tag fused to the C-terminus of the protein of interest. Purification tags are well known in the art. In some embodiments, the purification tag is a FLAG tag, a MYC tag, aHIS tag, or a green fluorescent protein (GFP).

In some embodiments, the one or more nucleic acid sequences further comprise a nucleic acid sequence comprising a universal primer sequence. In some embodiments, a universal primer sequence is located 5′ to the nucleic acid sequence encoding the protein of interest and a universal primer sequence is located 3′ to the nucleic acid sequence encoding the protein of interest.

In some embodiments, the nucleic acid sequence encoding the RNA-binding capsid protein comprises a nucleic acid sequence 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 4. In some embodiments, the nucleic acid sequence encoding the RNA stem-loop comprises a nucleic acid sequence 85%, 86%, 87%, 88%, 89%, 90%, 91%, 92%, 93%, 94%, 95%, 96%, 97%, 98%, 99%, or 100% identical to SEQ ID NO: 2 or SEQ ID NO: 3. In some embodiments, the nucleotide sequence encoding the MCP or RNA binding protein is codon optimized for expression in a mammalian cell. In some embodiments, the nucleotide sequence encoding the MCP or RNA binding protein is codon optimized for expression in any host cell of choice, including but not limited to, yeast cells, mammalian cells, insect cells, and bacterial cells.

In some embodiments, the plurality of different proteins of interest are representative of a proteome of interest. In some embodiments, the proteome of interest is the proteome of the cells. In some embodiments, the cells are Saccharomyces cerevisiae. In some embodiments, the proteome of interest is the cells are mammalian cells. In some embodiments, the cells are human cells.

In some embodiments, the determining further comprises normalizing the amount of mRNA detected to the amount of mRNA detected of non-specific functional controls. In some embodiments, the non-specific functional controls are proteins of interest represented in the plurality of proteins of interest but are not isolated by the biochemical assay. In some embodiments, a complex of proteins is purified, wherein the protein of interest is part of the complex.

In some embodiments, the nucleic acid sequence encoding the protein of interest comprises one or more deletions, insertions, or mutations as compared to its wild-type sequence as described herein. In some embodiment, the protein of interest encoded by the nucleic acid sequence comprises one or more deletions, insertions, or mutations as compared to its wild-type sequence as described herein.

In some embodiments, the nucleic acid sequence further comprises a terminator sequence operatively linked to the nucleotide sequence encoding the UMI and RNA stem-loop or cognate RNA sequence. In some embodiments, a UMI is a random sequence of nucleotides. In some embodiments, the UMI is a random sequence of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some embodiments, the UMI is a random sequence of 20 to 30 nucleotides. In some embodiments, the UMI is long enough to provide a unique sequence for all ORFs in an in vivo mRNA display library. In some embodiments, the second cassette is 50 to 100 nucleotides long. In some embodiments, each protein in the in vivo mRNA display library described herein is identified by a UMI.

### Method of Determining Protein-Protein Interactions

In some embodiments, the subject matter disclosed herein relates to detecting protein-protein interactions using an in vivo mRNA display library of proteins of interest. In some embodiments, the detection of protein-protein interactions includes utilizing at least one proximity-based method. In some embodiments, the proximity based methods are known in the art. In some embodiments, the proximity based method is proximity ligation.

In certain aspects, the invention provides a method of determining protein-protein interactions, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest; d) lysing the population of cells; e) incubating the lysate of step d); f) performing proximity ligation to generate a plurality of hybrid sequences comprising the mRNA sequence encoding a first protein of interest and a mRNA sequence encoding one or more additional proteins of interest; g) for each protein of interest, sequencing the hybrid sequence generated in step f); h) for each protein of interest, identifying the one or more additional proteins of interest encoded by each hybrid sequence; wherein the additional proteins of interest of the plurality of hybrid sequences are identified as forming a protein-protein interaction with the first protein of interest. In some embodiments, the incubating of step e) comprising mixing the lysate.

In certain aspects, the invention provides a method of determining protein-protein interactions, the method comprising: a) providing a population of cells, wherein each cell comprises one or more nucleic acid sequences encoding: a RNA-binding protein; a protein of interest; and a nucleotide sequence encoding a unique molecular identifier (UMI) sequence operably linked to a nucleotide sequence encoding an RNA stem-loop, wherein the nucleic acid sequence encoding the RNA-binding protein and protein of interest are operably linked so that they encode a fusion protein of the RNA-binding protein and protein of interest; and wherein the nucleotide sequence encoding the UMI and the RNA stem-loop are operably linked so that a mRNA encoding the protein of interest comprises the UMI sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds to the RNA stem-loop present on the RNA sequence encoding the UMI sequence and the RNA stem-loop sequence; d) lysing the population of cells; e) incubating the lysate of step d); f) performing proximity ligation to generate a plurality of hybrid sequences comprising the RNA sequence encoding a first UMI and a RNA sequence encoding one or more additional UMIs; g) for each hybrid sequence generated in step f), sequencing the one or more UMIs in the hybrid system; h) determining the protein of interest associated with each UMI of each hybrid sequence in f); wherein the proteins of interest associated with a hybrid sequence are identified as forming a protein-protein interaction. In some embodiments, the incubating of step e) comprising mixing the lysate.

In some embodiments, a hybrid sequence is generated by incorporating two or more RNA sequences which may comprise a UMI, and which are localized in close proximity. In some embodiments, the hybrid system is generated using at least one proximity based methods. In some embodiments, the proximity based method is proximity ligation. In some embodiments, each UMI or ORF in the hybrid sequence is part of a in vivo mRNA display cassette. In some embodiments, each UMI in the hybrid sequence uniquely identifies a protein of interest from an in vivo mRNA display library. In some embodiments, the proximity of the sequences included in the hybrid sequence is due to specific interactions between proteins displayed in the mRNA display cassette. In some embodiments, at least one UMI from the hybrid sequence is read to determine the protein it identifies. In some embodiments, incorporation of UMI into a hybrid sequence is indicative of an interaction between the proteins of interest identified by the incorporated UMI. In some embodiments, the detected protein-protein interactions are quantified.

In some embodiments, the hybrid RNA sequence containing the sequences from the two ORFs or the UMI representing them, are reverse transcribed to DNA and DNA sequenced.

### Method of Determining Protein-DNA or Protein-RNA Interactions

In some embodiments, the subject matter disclosed herein relates to detecting protein-DNA interactions using an in vivo mRNA display. In some embodiments, the subject matter disclosed herein relates to detecting protein-RNA interactions using an in vivo mRNA display.

In some embodiments, the detection of protein-DNA or protein-RNA interactions includes utilizing at least one proximity-based method. In some embodiments, the proximity based method is proximity ligation.

In certain aspects the invention provides a method of determining protein-DNA or protein-RNA interactions, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;

wherein the population of cells comprise nucleic acid sequences encoding a plurality of different proteins of interest; b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the protein of interest; d) lysing the population of cells; e) performing a biochemical assay on a portion of the lysate of step d) to generate an enriched lysate; wherein the biochemical assay comprises enriching a DNA or RNA molecule of interest; f) detecting the amount of mRNA encoding each protein of interest and comprising the RNA stem-loop that is present in the lysate of step d); g) detecting the amount of mRNA encoding each protein of interest and comprising the RNA stem-loop that is present in the enriched lysate of step e); and h) for each protein of interest, comparing the amount of mRNA detected in step d) and step g), wherein a protein of interest is determined to be enriched under the biochemical assay conditions if an increased level of mRNA is detected in step g) compared to step d) or wherein a protein of interest is determined to be depleted under the biochemical assay conditions is a decreased level of mRNA is detected in step g) compared to step d). In some embodiments, a population of DNA or RNA molecules to the lysate of step d). In some embodiments, the population of DNA molecules or RNA molecules added comprise a tag. In some embodiments, the tag comprises an affinity tag, a chemical modification, or an aptamer. In some embodiments, the tag is used to perform the enriching of step e). In some embodiments, each DNA molecule of the added population of DNA molecules is the same. In some embodiments, the added population of DNA molecules comprises a population of different DNA molecules. In some embodiments, each RNA molecule of the added population of RNA molecules is the same. In some embodiments, the added population of RNA molecules comprises a population of different RNA molecules.

In certain aspects the invention comprises a method of determining protein-DNA or protein-RNA interactions, the method comprising: a) providing a population of cells, wherein each cell comprises one or more nucleic acid sequences encoding: a RNA-binding protein; a protein of interest; a unique molecular identifier (UMI); and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the UMI comprises the cognate RNA sequence; wherein the population of cells comprise nucleic acid sequences encoding a plurality of different proteins of interest;

b) expressing the said one or more nucleic acid sequences; c) incubating the cells, thereby producing a population of cells comprising an in vivo mRNA display, wherein the expressed RNA-binding protein-protein of interest fusion protein binds to the cognate RNA sequence present on the mRNA sequence encoding the UMI; d) lysing the population of cells; e) performing a biochemical assay on a portion of the lysate of step d) to generate an enriched lysate; wherein the biochemical assay comprises enriching a DNA or RNA molecule of interest;
f) detecting the amount of mRNA encoding each UMI and comprising the RNA stem-loop that is present in the lysate of step d); g) detecting the amount of mRNA encoding each UMI and comprising the RNA stem-loop that is present in the enriched lysate of step e); and h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f). In some embodiments, a population of DNA or RNA molecules to the lysate of step d). In some embodiments, the population of DNA molecules or RNA molecules added comprise a tag. In some embodiments, the tag comprises an affinity tag, a chemical modification, or an aptamer. In some embodiments, the tag is used to perform the enriching of step e). In some embodiments, each DNA molecule of the added population of DNA molecules is the same. In some embodiments, the added population of DNA molecules comprises a population of different DNA molecules. In some embodiments, each RNA molecule of the added population of RNA molecules is the same. In some embodiments, the added population of RNA molecules comprises a population of different RNA molecules.

In certain aspects, the invention provides a method of determining protein-DNA or protein-RNA interactions, the method comprising: a) providing a population of cells, wherein each cell comprises one or more nucleic acid sequences encoding: a RNA-binding protein; a protein of interest; and a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;

wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest; d) lysing the population of cells; e) adding a population of DNA or RNA molecules to the lysate of step d); f) incubating the mixture of lysate and a population of DNA or RNA molecules of step e); g) performing proximity ligation to generate a plurality of hybrid sequences comprising the mRNA sequence encoding a protein of interest or a cDNA copy thereof and one or more of the DNA or RNA molecules; h) for each protein of interest, sequencing the hybrid sequence generated in step g); i) for each protein of interest, identifying the one or more DNA or RNA molecules of each hybrid sequence; wherein the one or more DNA or RNA molecules of the plurality of hybrid sequences are identified as forming an interaction with the protein of interest. In some embodiments, the population of DNA molecules or RNA molecules added in step e) comprise a tag. In some embodiments, the tag comprises an affinity tag, a chemical modification, or an aptamer. In some embodiments, each DNA molecule of the population of DNA molecules is the same. In some embodiments, the population of DNA molecules comprises a population of different DNA molecules. In some embodiments, each RNA molecule of the population of RNA molecules is the same. In some embodiments, the population of RNA molecules comprises a population of different RNA molecules.

In certain aspects, the invention provides a method of determining protein-DNA or protein-RNA interactions, the method comprising: a) providing a population of cells, wherein each cell comprises one or more nucleic acid sequences encoding: a RNA-binding protein; a protein of interest; and a nucleotide sequence encoding a unique molecular identifier (UMI) sequence operably linked to a nucleotide sequence encoding an RNA stem-loop; wherein the nucleic acid sequence encoding the RNA-binding protein and protein of interest are operably linked so that they encode a fusion protein of the RNA-binding protein and protein of interest; and wherein the nucleotide sequence encoding the UMI sequence and the RNA stem-loop are operably linked so that a mRNA encoding the RNA stem-loop comprises the UMI sequence; wherein the population of cells comprise nucleic acid sequences encoding a plurality of different proteins of interest; b) expressing the said one or more nucleic acid sequences; c) incubating the cells, thereby producing a population of cells comprising an in vivo mRNA display library, wherein the expressed RNA-binding protein-protein of interest fusion protein binds with high affinity to the RNA stem-loop on the nucleotide sequence encoding the UMI sequence and the RNA stem-loop; d) lysing the population of cells; e) adding a population of DNA or RNA molecules to the lysate of step d); f) incubating the mixture of lysate and a population of DNA or RNA molecules of step e); g) performing proximity ligation to generate a plurality of hybrid sequences comprising the mRNA sequence encoding the UMI or a cDNA copy thereof and one or more of the DNA or RNA molecules; h) for each hybrid sequence generated in step g), sequencing the UMI and the one or more DNA or RNA sequences of the hybrid sequence; g) determining the protein of interest associated with each UMI of each hybrid sequence in h); wherein the one or more DNA or RNA molecules of the plurality of hybrid sequences are identified as forming an interaction with the protein of interest. In some embodiments, the population of DNA molecules or RNA molecules added in step e) comprise a tag. In some embodiments, the tag comprises an affinity tag, a chemical modification, or an aptamer. In some embodiments, each DNA molecule of the population of DNA molecules is the same. In some embodiments, the population of DNA molecules comprises a population of different DNA molecules. In some embodiments, each RNA molecule of the population of RNA molecules is the same. In some embodiments, the population of RNA molecules comprises a population of different RNA molecules.

In some embodiments, a hybrid sequence is generated incorporating (i) one or more RNA sequence which may comprise a UMI and (ii) one or more DNA sequences from a population of DNA sequences. In some embodiments, the hybrid sequence is generated using proximity ligation or other proximity based methods. In some embodiments, the one or more RNA sequence which may comprise a UMI and the one or more DNA sequences from a population of DNA sequences were localized in close proximity prior to ligation. In some embodiments, a hybrid sequence is generated incorporating (i) one or more RNA sequence which may comprise a UMI and (ii) one or more RNA sequences from a population of RNA sequences. In some embodiments, the hybrid sequence is generated using proximity ligation or other proximity based methods. In some embodiments, the one or more RNA sequence which may comprise a UMI and the one or more RNA sequences from a population of RNA sequences were localized in close proximity prior to ligation. In some embodiments, each UMI or ORF in a hybrid sequence is part of a in vivo mRNA display cassette. In some embodiments, each UMI in a hybrid sequence uniquely identifies a protein of interest from an in vivo mRNA display library. In some embodiments, at least one UMI from the hybrid sequence is read to determine the protein it identifies. In some embodiments, the DNA or RNA sequence incorporated in the hybrid sequence is sequenced. In some embodiments, the DNA or RNA sequence incorporated in the hybrid sequence is sequenced using methods known in the art. In some embodiments, incorporation of a UMI into a hybrid sequence is indicative of an interaction between the protein of interest identified by the incorporated UMI and the DNA or RNA molecule incorporated into the same hybrid sequence. In some embodiments, the protein-DNA or protein-mRNA interactions are quantified.

In some embodiments, the hybrid sequence containing the sequences from the ORF, or the UMI representing it, and RNA interacting with the protein encoded by the ORF is reverse transcribed to DNA and DNA sequenced.

In some embodiments, the mRNA encoding the ORF, or the UMI representing it, is reverse transcribed and proximity ligated to a DNA molecule interacting with the protein encoded by the ORF to generate the hybrid sequence.

### Method of Detecting Phosphorylation Dynamics of the Proteome

In some embodiments, the subject matter disclosed herein relates to detection and/or quantification of phosphorylation dynamics of the proteome. In some embodiments, the phosphorylation dynamics are global phosphorylation dynamics. In some embodiments, the detection includes using phosphor-specific antibodies. In some embodiments, the phosphorylation dynamics of the proteome are in response to at least one environmental stimulus. In some embodiments, the phosphorylation dynamics of the proteome are in response to at least one genetic perturbation. In some embodiments, the genetic perturbation results in altered kinase activity. In some embodiments, the genetic perturbation results in altered phosphatase activity.

### EXAMPLES

The following examples illustrate the present invention, and are set forth to aid in the understanding of the invention, and should not be construed to limit in any way the scope of the invention as defined in the statements of the invention which follow thereafter.

The Examples described below are provided to illustrate aspects of the present invention and are not included for the purpose of limiting the invention.

### Example 1—In Vivo mRNA Display: Large-Scale Proteomics by Next Generation Sequencing

Large-scale proteomic methods are essential for the functional characterization of proteins in their native cellular context. However, proteomics has lagged far behind genomic approaches in scalability, standardization and cost. The subject matter described herein relates to in vivo mRNA display, a technology that converts a variety of proteomics applications into a DNA sequencing problem. In vivo expressed proteins are coupled with their encoding mRNAs via a high-affinity stem-loop RNA binding domain interaction, enabling high-throughput identification of proteins with high sensitivity and specificity by next-generation DNA sequencing. The subject matter described herein also relates to a high-coverage in vivo mRNA display library of the S. cerevisiae proteome and its potential for characterizing subcellular localization and interactions of proteins expressed in their native cellular context. In vivo mRNA display libraries can circumvent the limitations of mass spectrometry-based proteomics and leverage the exponentially improving cost and throughput of DNA-sequencing to systematically characterize native functional proteomes.

Cellular proteins act in concert with each other to achieve a diverse set of functions through protein-protein interactions, regulatory interactions, post-translational modifications, and subcellular localization. Proteomic technologies allow for the dissection of the functional roles of proteins in the context of biological processes, cellular compartments, and metabolic/signaling pathways¹. A comprehensive view of this complex proteomic landscape depends on the ability to reliably identify and characterize proteins in their native physiological contexts with precision and specificity². Current high throughput approaches utilize mass spectrometry coupled with affinity purification³ or sub-fractionation⁴, as well as reporter assays such as the yeast two-hybrid⁵⁻⁷, FRET⁸ and protein complementation⁹⁻¹¹. Additionally, methods developed under the category of “spatial proteomics” can label and purify proteins within a certain radius of a chosen bait¹²⁻¹⁷. Although such studies have provided a snapshot of the cellular proteome in many contexts¹⁷⁻²⁴, the picture is far from comprehensive due to the vast space of possible interactions, the diverse roles played by proteins in different cellular contexts, as well as the transient nature of many interactions²⁵⁻²⁷. Meanwhile, Next-Generation Sequencing (NGS) has ushered genomics into a new age due to its low cost, precision, accuracy and capacity for massive multiplexing. Furthermore, many biochemical assays have been adapted to take advantage of NGS by mapping functional assays to a DNA sequencing readout. Such applications include Hi-C²⁸, ATAC-seq²⁹, bisulfite sequencing³⁰ and others. However, functional proteomics have not yet tapped into NGS's full potential at a similar scale and fashion^{31,32}.

The subject matter described herein introduces in vivo mRNA display, a technology that enables diverse proteomics applications using NGS as the readout. A variety of existing display technologies create a link between genotype and phenotype whereby a protein or peptide is linked to its encoding nucleic-acid. For example, in phage display, the nucleic acid encoding the capsid displayed peptide is contained within the phage³³. The resulting collection of displayed peptides can be used for the in vitro characterization of protein interactions, protein engineering and selection of human antibody fragment libraries³⁴⁻³⁶. Alternative in vitro methods linking nucleotide information to phenotype include mRNA display^{37,38}, ribosome display³⁹, and yeast display⁴⁰. In the past decade, many of these technologies have been coupled with NGS⁴¹⁻⁴⁴. More recently, a method called ACAP-seq⁴⁵ converts in vitro interactions of nascent polypeptides and their polyribosomes to RNA sequencing. Another approach adapted DNA sequencing chips to immobilize collections of DNA-RNA-protein complexes and carry out fluorescence-based functional assays on the chip⁴⁶. Despite their diverse utility, these existing display technologies are limited to analysis of proteins in vitro, significantly limiting their physiological relevance due to lack of appropriate cellular context, in vivo post-translational modifications and even proper folding states⁴¹.

In one embodiment, the subject matter described herein relates to engineering a scalable display technology that functions in vivo. The high-affinity interaction between the MS2 bacteriophage capsid protein and its cognate RNA stem-loop^{47,48} was co-opted. This interaction was previously utilized as a reporter system to track mRNA molecules in living cells in a variety of organisms⁴⁹. In these assays, tandem copies of the stem-loop sequence were inserted adjacent to the monitored gene, which enables the detection of its mRNA through the interaction of the stem-loop with a fluorescent protein fused to the MS2 coat protein (MCP)^{50,51}. In one embodiment, the subject matter disclosed herein relates to modifying the MS2 tagging system in order to associate a translated protein with its own mRNA. The MS2 coat protein (MCP) was fused to the N-terminus of a target protein while the cognate RNA stem-loop was introduced downstream of the gene, establishing a direct link between gene and protein (FIG. 1A). In contrast to in vitro display technologies, assayed proteins are expressed, processed, and tagged in vivo in their relevant cellular contexts. This approach, termed in vivo mRNA display, identifies proteins in a variety of in vivo functional assays using nucleic-acid sequencing as the readout.

In Vivo mRNA Display for Protein Identification

To demonstrate in vivo mRNA display, an episomally expressed inducible construct, expressing an MCP-ORF fusion was generated. This fusion includes a short polypeptide purification tag and is followed by a single copy of the 19 nt stem-loop (SL)⁴⁷ such that, upon translation, the fusion product binds to its encoding mRNA (FIG. 1A). Following transformation, each strain contains a single species of the in vivo mRNA display construct corresponding to a single displayed protein, which interacts with its cellular context independently from all the other species in the library (FIG. 1). Induced cells can be assayed according to the desired biochemical assay (e.g. immunoprecipitation of a bait) which should preserve the RNA-protein interaction (FIG. 1C). The enrichment/depletion of each ORF sequence can be quantified by comparing their abundance in isolated RNA before and after the assay.

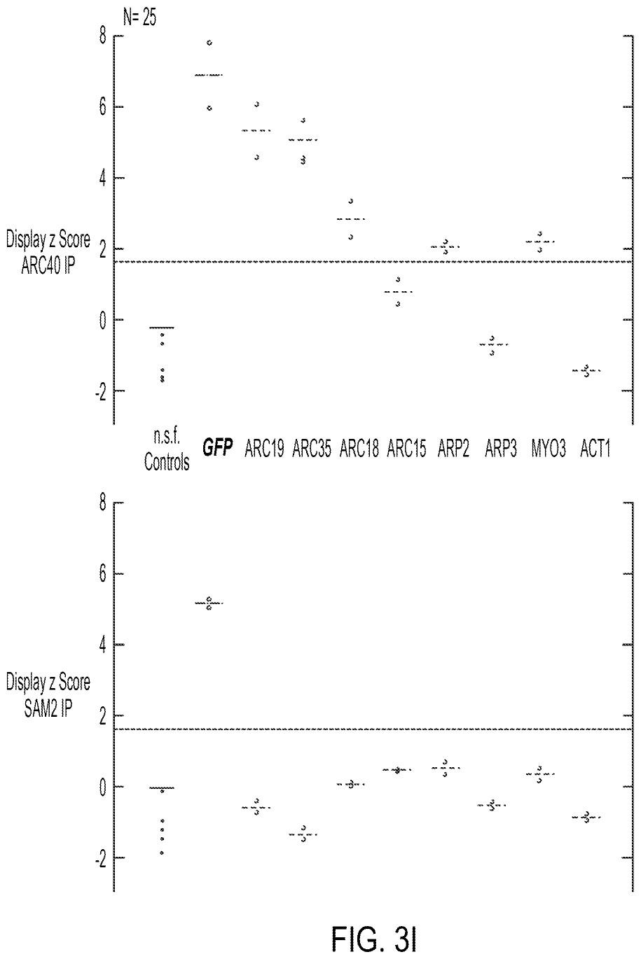

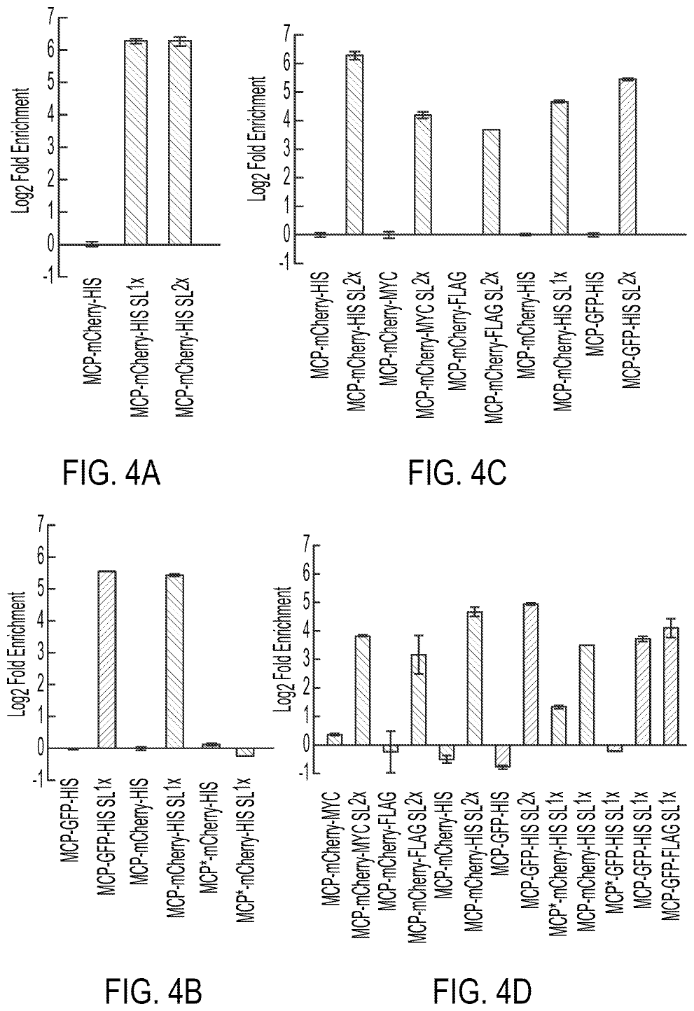

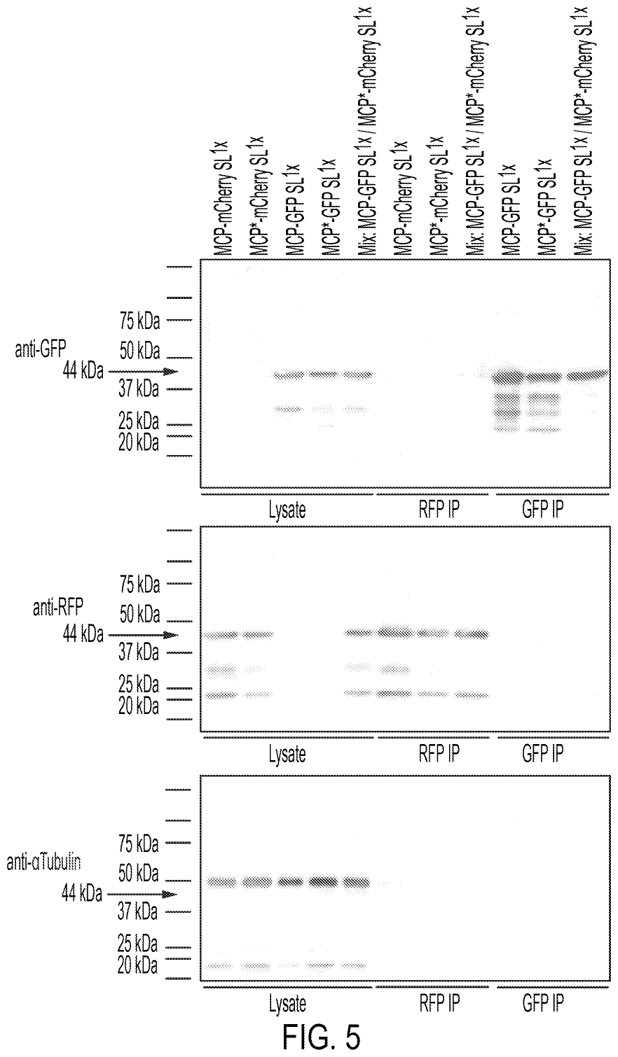

To demonstrate the propensity of an mRNA displayed protein to stably interact with its encoding mRNA, a set of strains expressing MCP fluorescent protein fusions was constructed. Each fusion protein was immunoprecipitated using magnetic beads that specifically recognize each construct (FIG. 1D, see Methods). Immunoprecipitation of the target protein co-purifies its self-identifying mRNA with an enrichment of 8-fold (P<0.01) relative to the input lysate as measured by RT-PCR over native housekeeping mRNAs. In contrast, a defective capsid construct, MCP* (N55D, K57E)⁴⁸, shows no enrichment of its respective mRNA upon purification. Similarly, deleting the downstream stem-loop also removes the enrichment (FIG. 4).

The precision with which a displayed protein-mRNA complex can be isolated in the presence of other displayed proteins was assessed. Co-transformation of two in vivo display constructs into yeast, one expressing a GFP, and the other a mCherry fusion, results in a mixed population of yeast cells each expressing one or the other. A significant enrichment of the GFP mRNA compared to mCherry mRNA when purifying GFP using anti-GFP magnetic beads from the mixed population (FIG. 1E, ˜11-fold, P=0.001), and vice versa for RFP (˜5-fold, P=0.016) was observe. Therefore, in vivo mRNA display proteins in one-on-one competitive assays were correctly identify by comparing the enrichment of mRNA levels to each other.

Discriminative Ability of In Vivo mRNA Display for High-Throughput Protein Identification

In order to systematically determine the sensitivity and specificity of in vivo mRNA display, a mix of three in vivo display libraries was constructed consisting of a few hundred distinct yeast S. cerevisiae proteins⁵². Each library carried a different C-terminal purification tag (FLAG, MYC and HIS), and was transformed into a haploid yeast strain. The purification tags were used to specifically isolate each protein subpopulation.

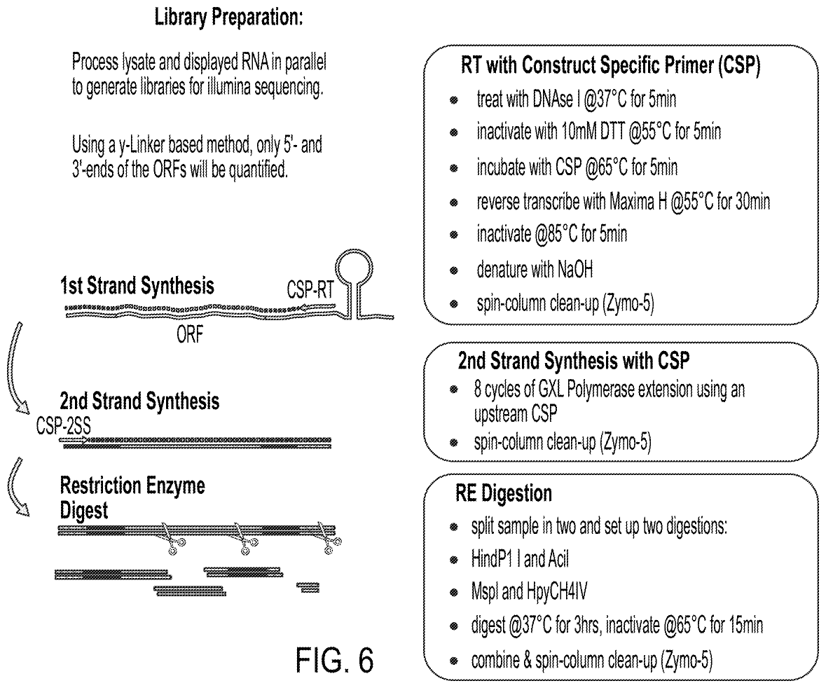

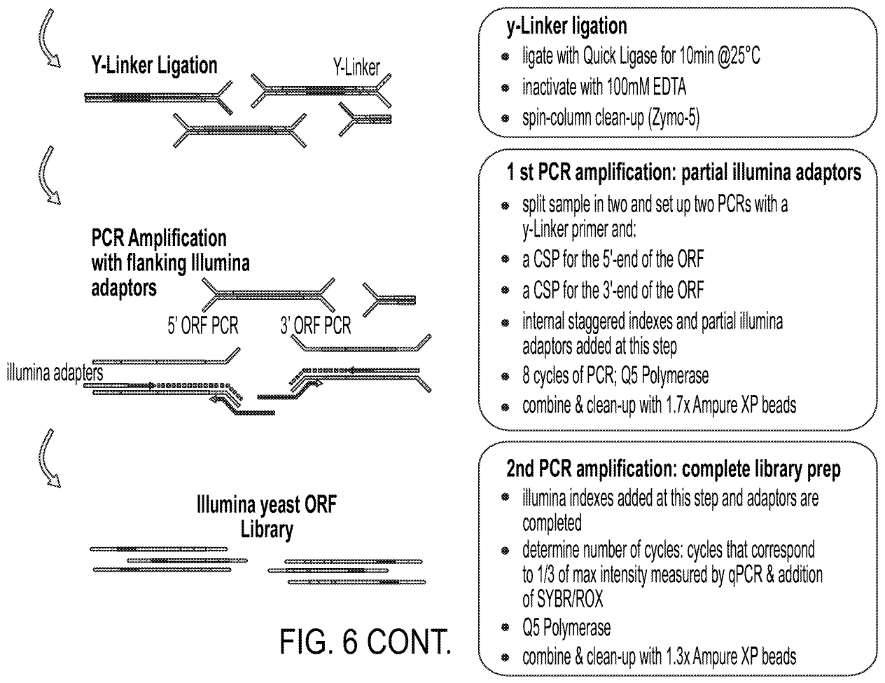

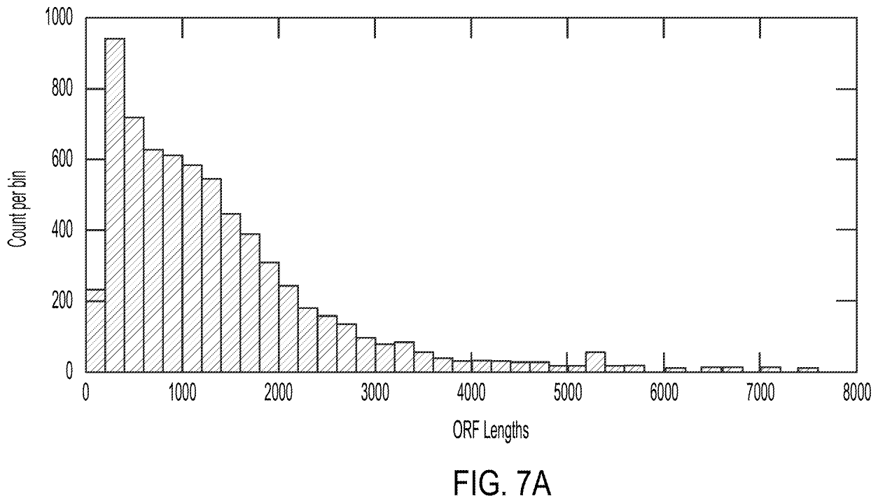

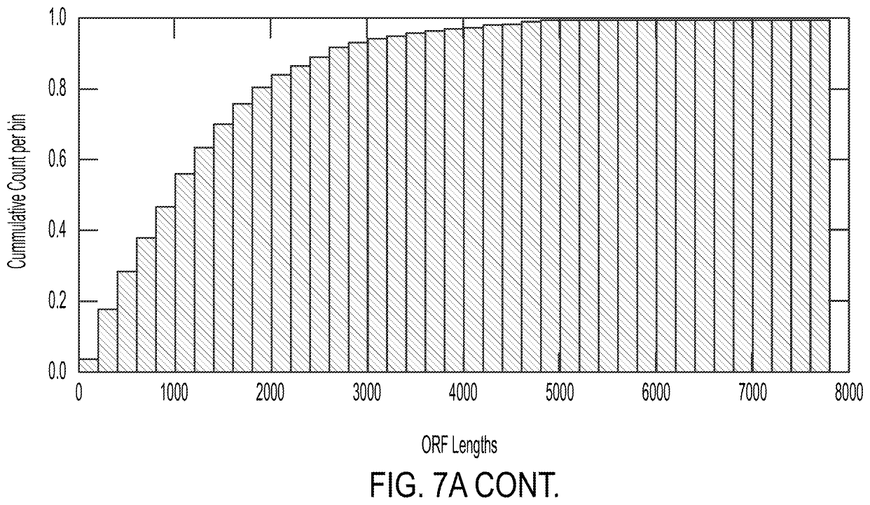

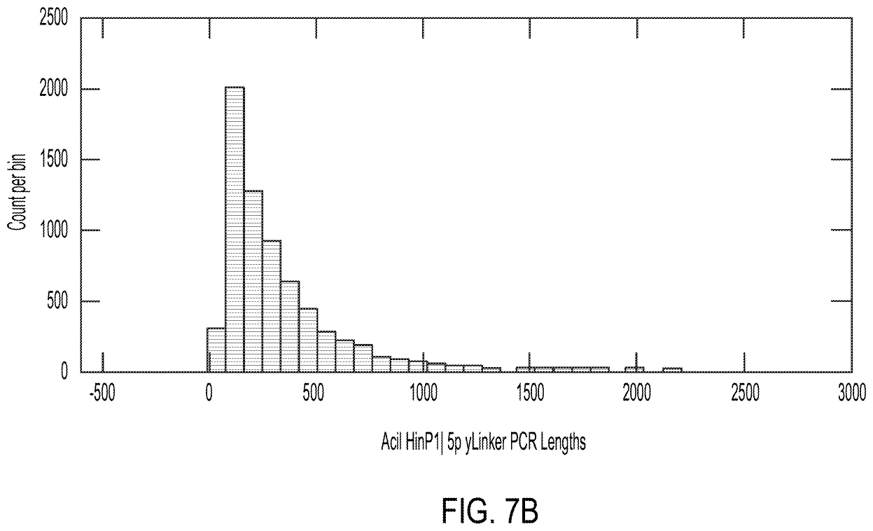

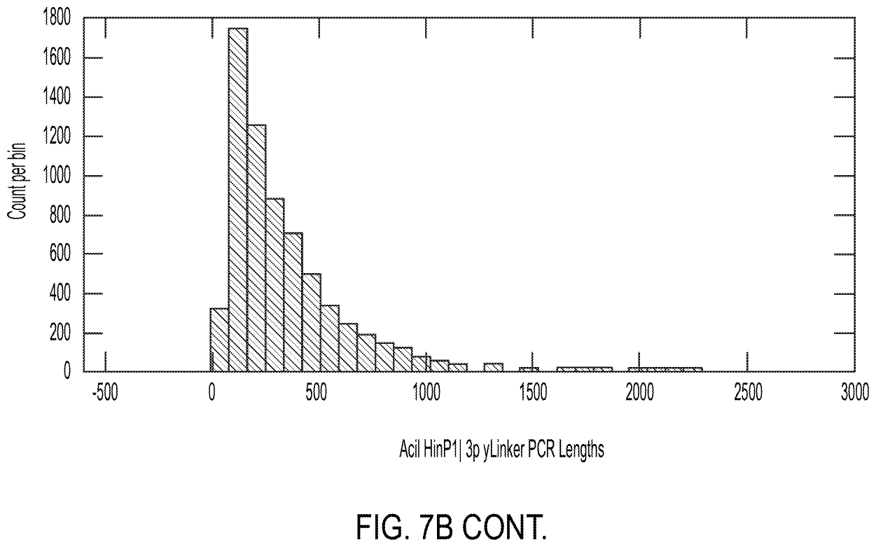

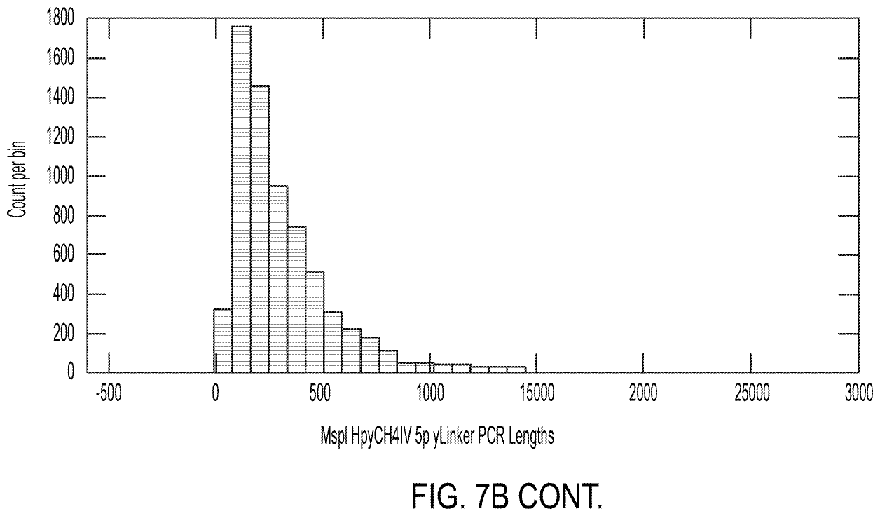

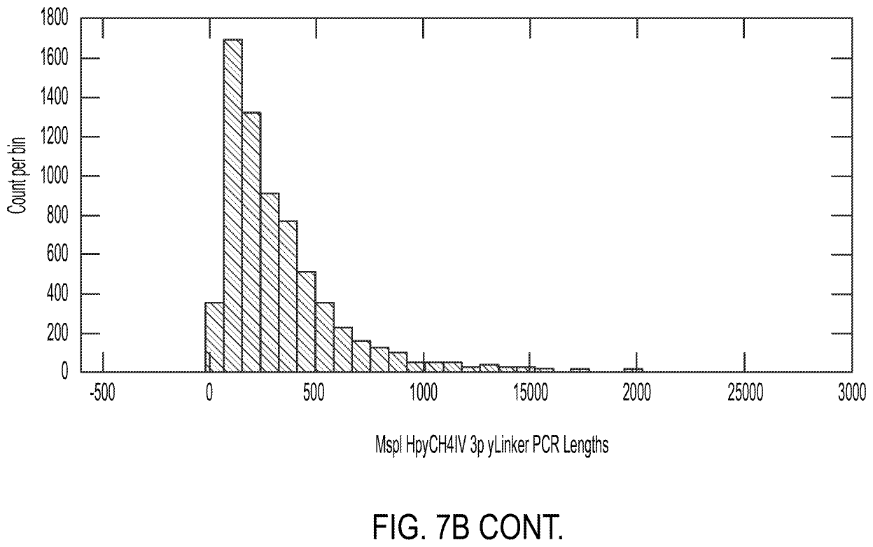

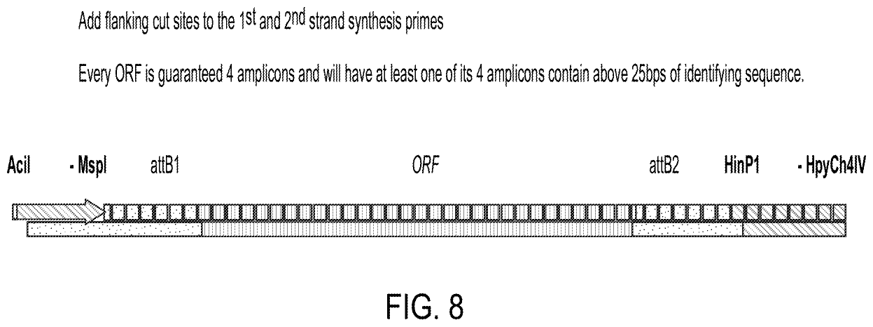

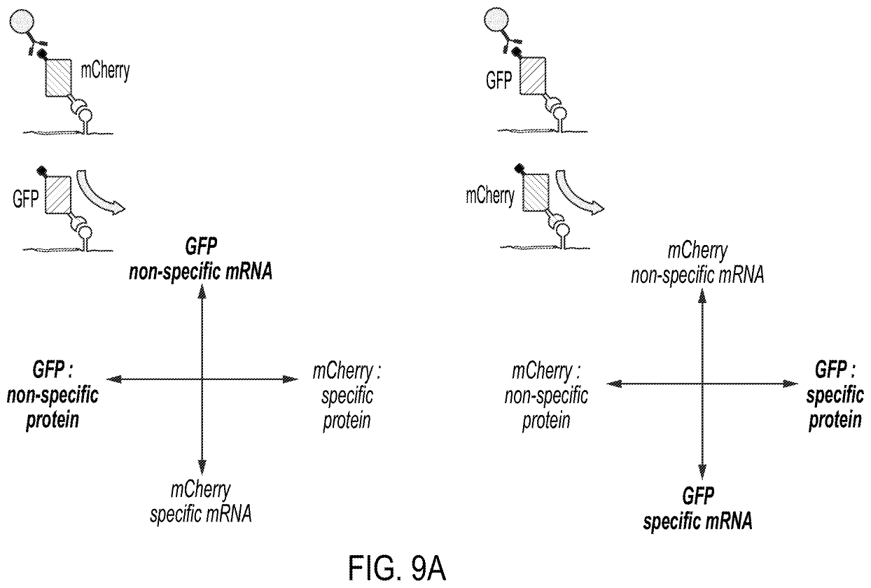

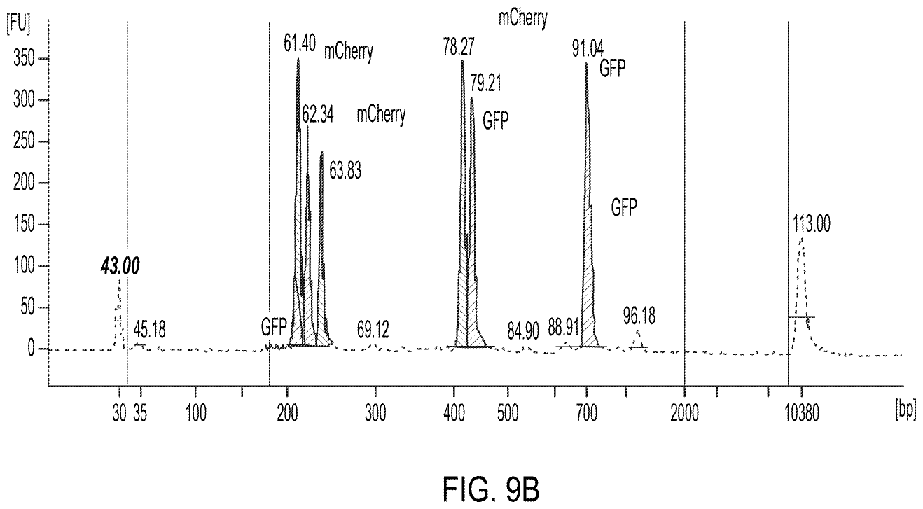

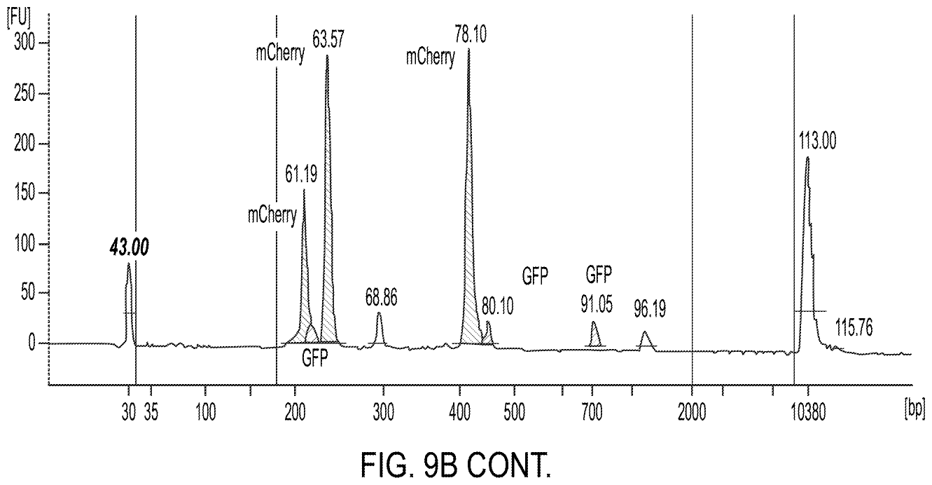

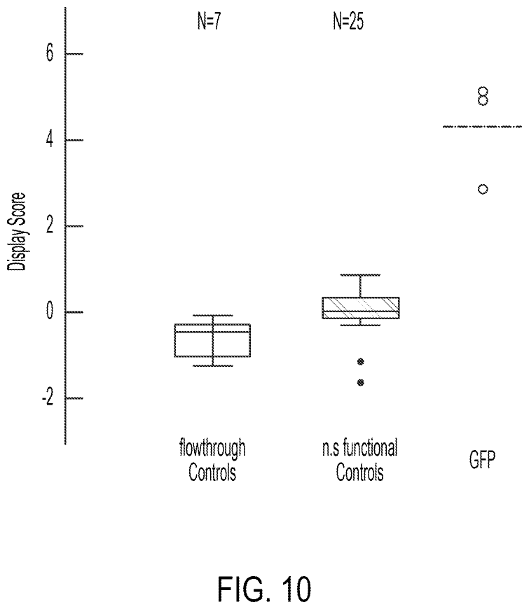

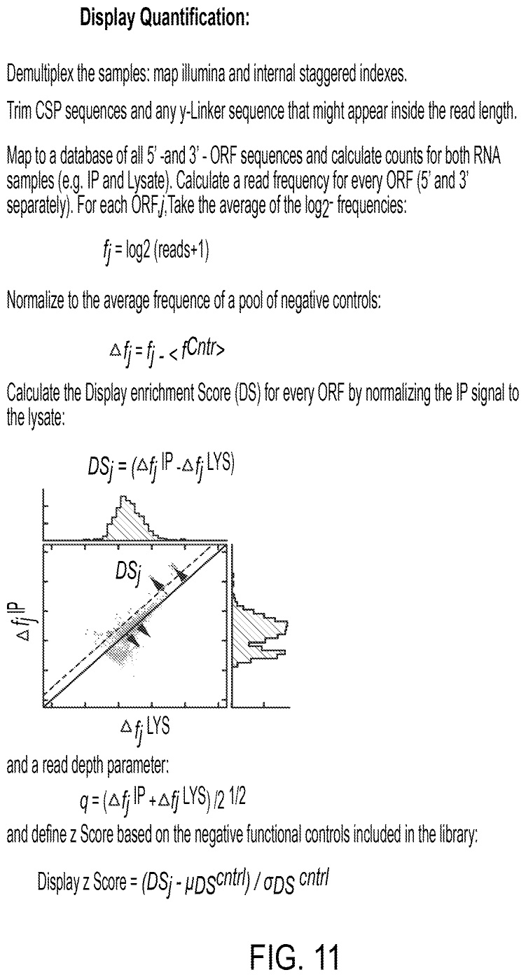

In order to quantify the frequency of each displayed ORF, a sequencing preparation protocol compatible with NGS (FIGS. 6-8, Methods) was designed. In brief, RNA was isolated from starting and purified protein samples. The library mRNAs were processed utilizing universal sequences flanking the ORF of each construct and Illumina adapters were added to the fragments corresponding to the 5′ and 3′ ends of each ORF, allowing to quantify frequencies with a minimal number of reads. Frequencies of fragments in the starting sample were compared to the frequencies from the isolated protein samples, and normalized to the frequencies of non-specific functional controls (FIGS. 9-11). The non-specific functional controls are a set of constructs that display their mRNA, but are not isolated in a given assay (Methods). For every ORF, a relative enrichment, termed Display Score (DS^{j}), was calculated. Additionally, a z Score and a significance value for the DS of each ORF were calculated from the distribution of the non-specific functional controls (FIG. 11, Methods).

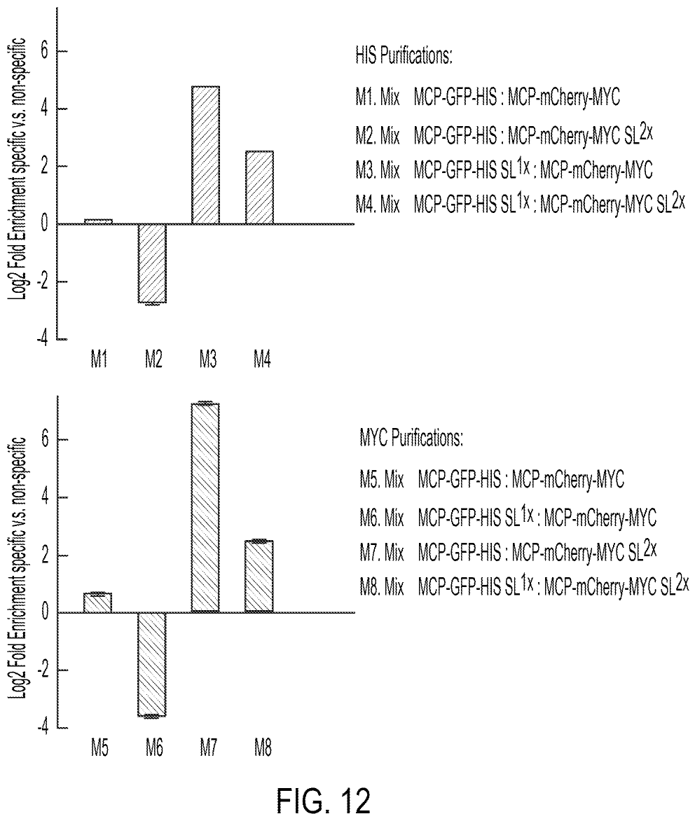

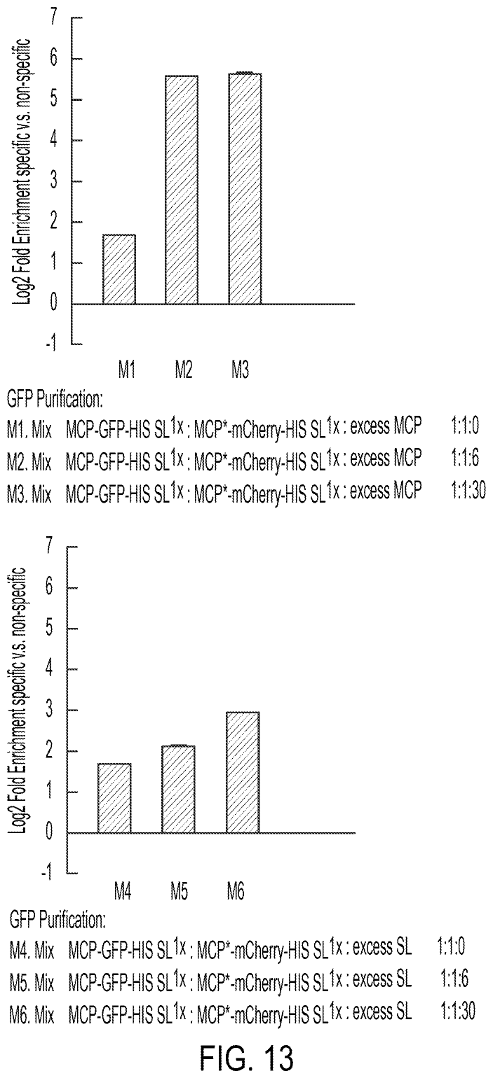

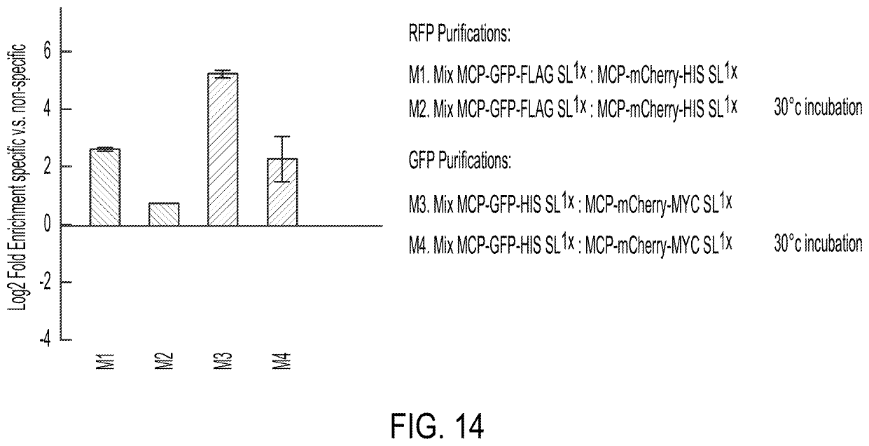

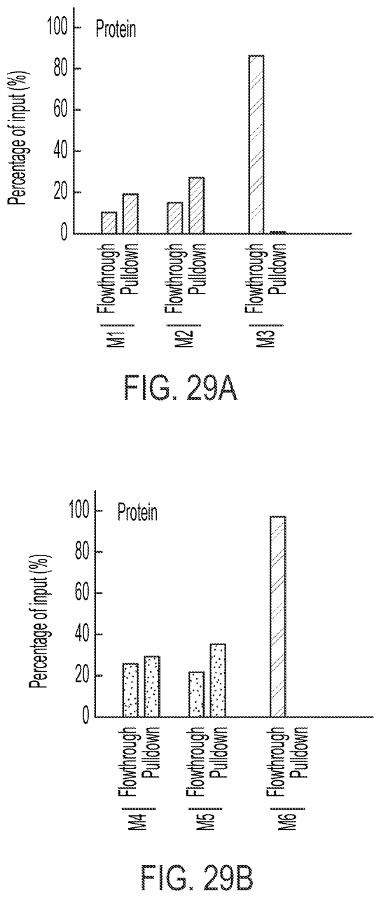

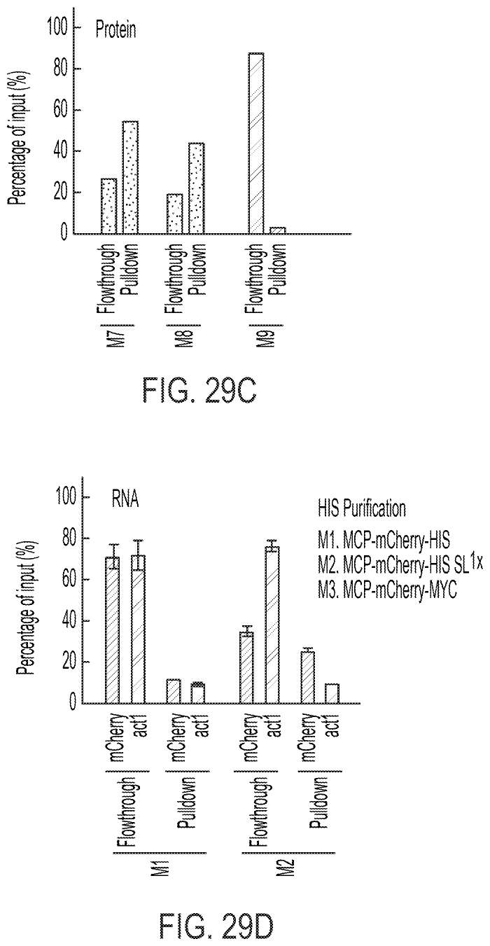

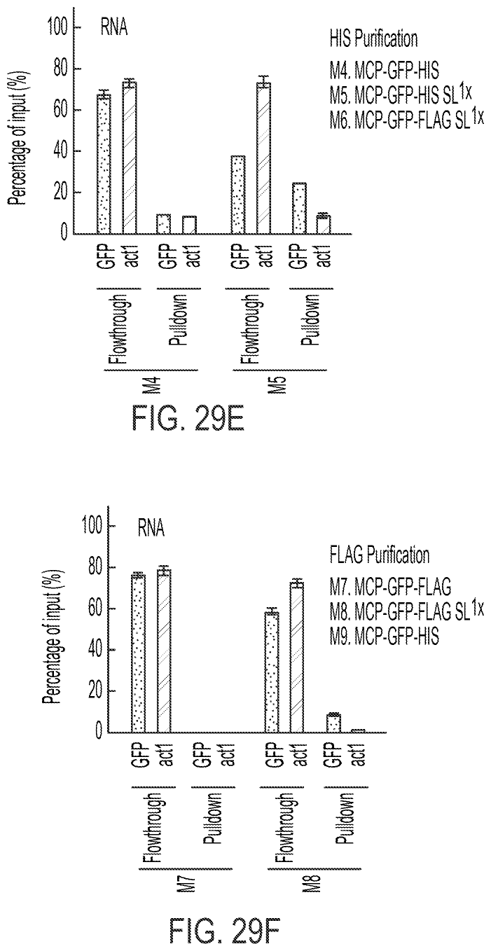

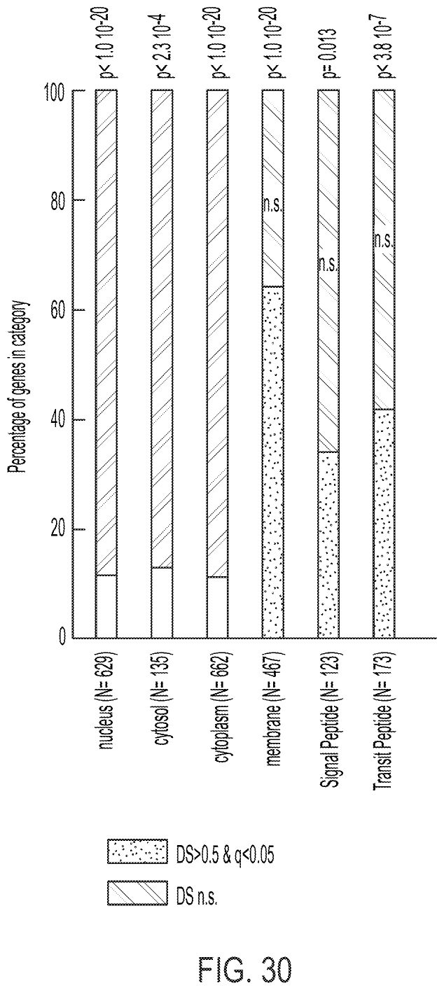

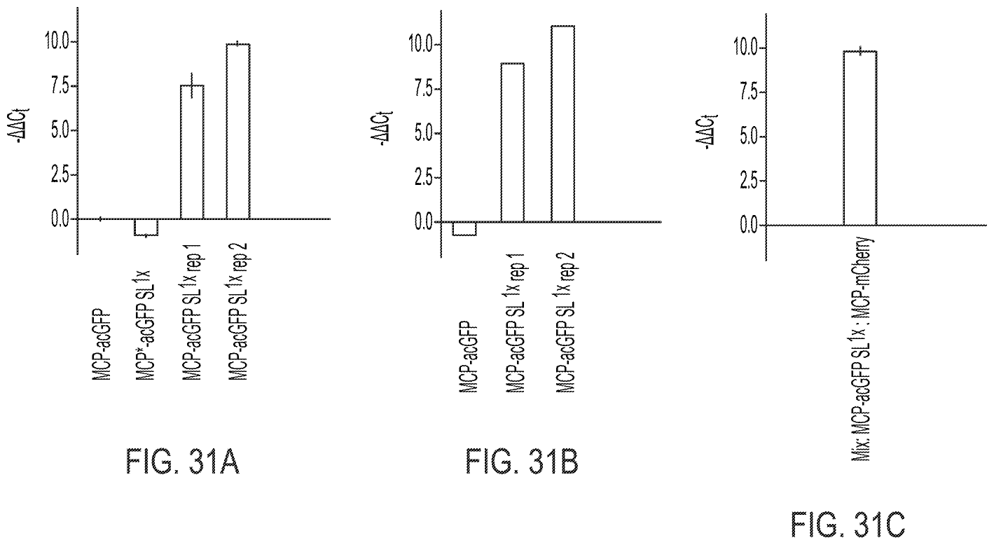

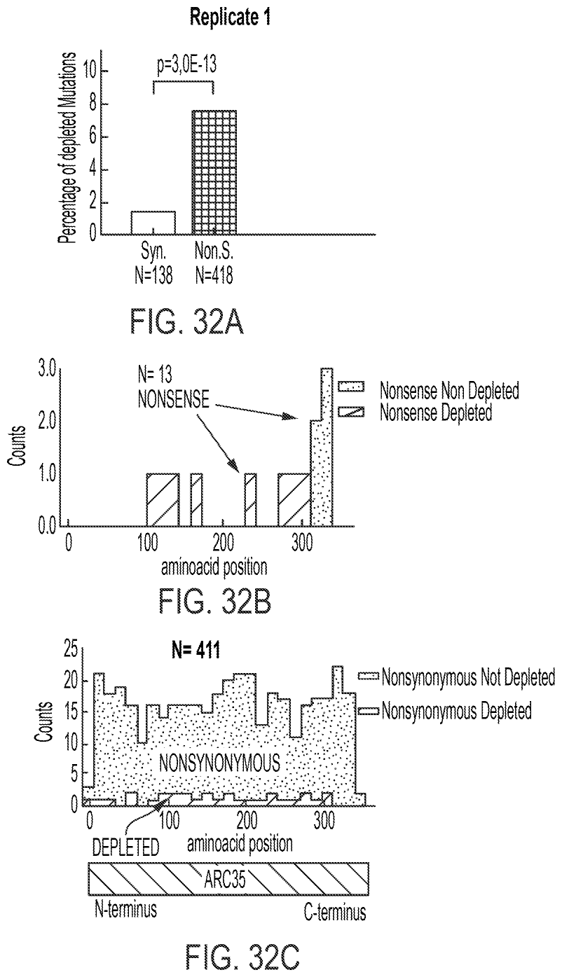

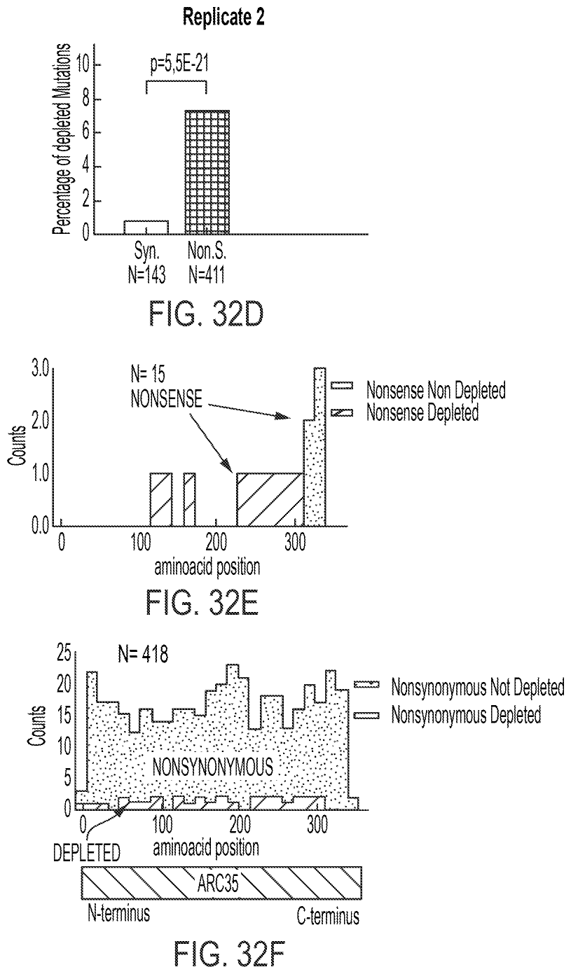

Since unbound capsids and stem-loops are free to interact with non-specific partners post lysis, they could compromise precision (FIG. 12 and FIGS. 29A-F). Therefore, an excess of capsid protein was provided in order to titrate any non-specific interactions (FIG. 13). Moreover, lysis and all purification steps were performed at 4° C. in order to minimize likelihood of partner exchanges due to possible disassociation of mRNA and MCP at higher temperatures (FIG. 14).

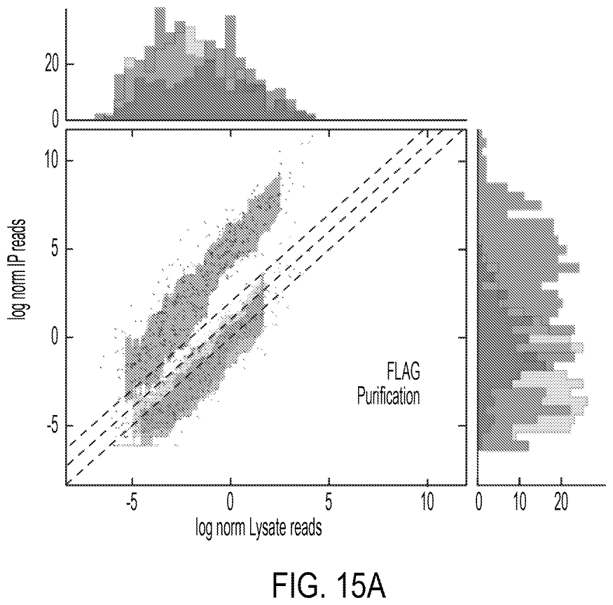

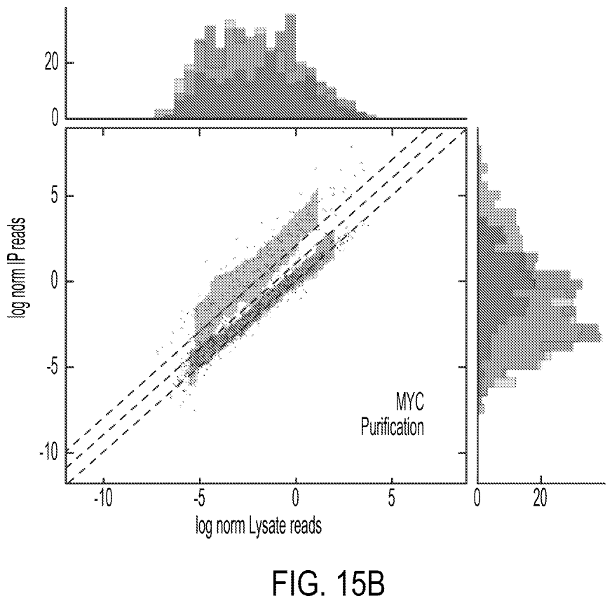

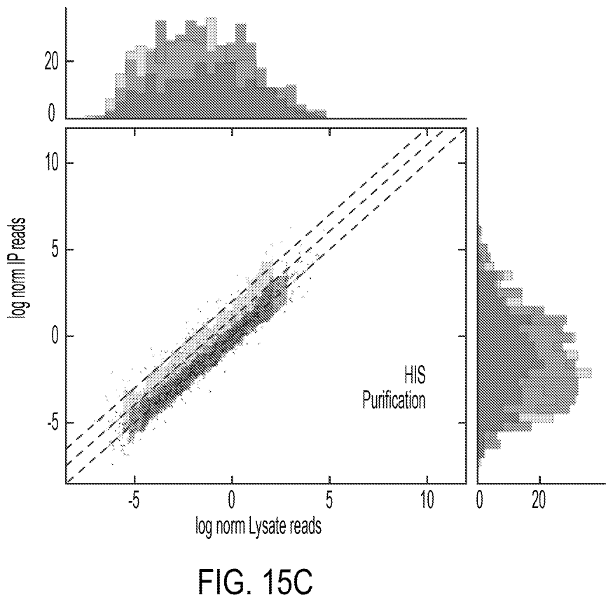

When using anti-FLAG beads to purify the FLAG tagged proteins from the mixed population, a substantial enrichment of the ORF mRNAs from the FLAG library was observed in the purified sample with respect to the lysate (FIG. 2A), while ORFs in the MYC and HIS tagged libraries were not enriched. Three separate purifications were conducted for each tag from the mixed population and the Display Scores for the ORFs in each library were quantified (FIGS. 2B-C, FIG. 15). The Display Score for each ORF was used to classify proteins as members of the immuno-precipitated population, resulting in the receiver operating curves in FIG. 2C. The high values for the area under the curve (AUC=0.98, 0.96 and 0.77 for FLAG, MYC and HIS respectively; FIG. 2C) demonstrate that in vivo mRNA display classified proteins to the correct population while maintaining low false positive rates. Although all three assays demonstrate relatively high discriminative ability, the FLAG and MYC purifications perform better than HIS suggesting a higher background during the IMAC based isolation of histidine-tagged proteins.

An In Vivo mRNA Display Library for Exploration of Yeast Proteomics

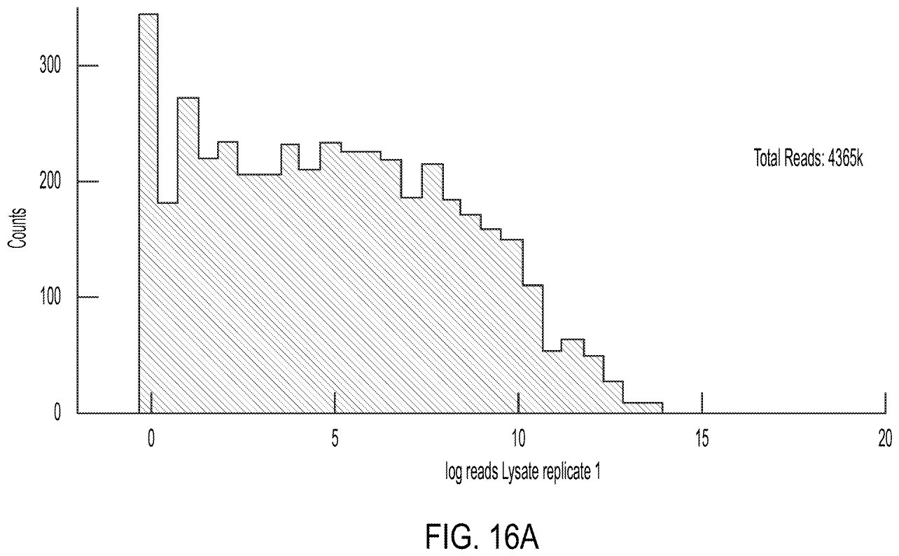

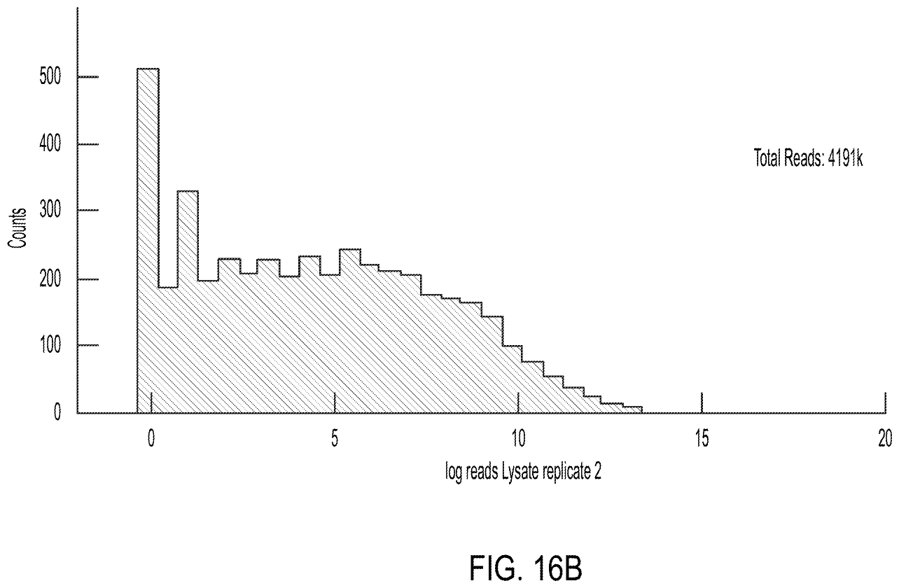

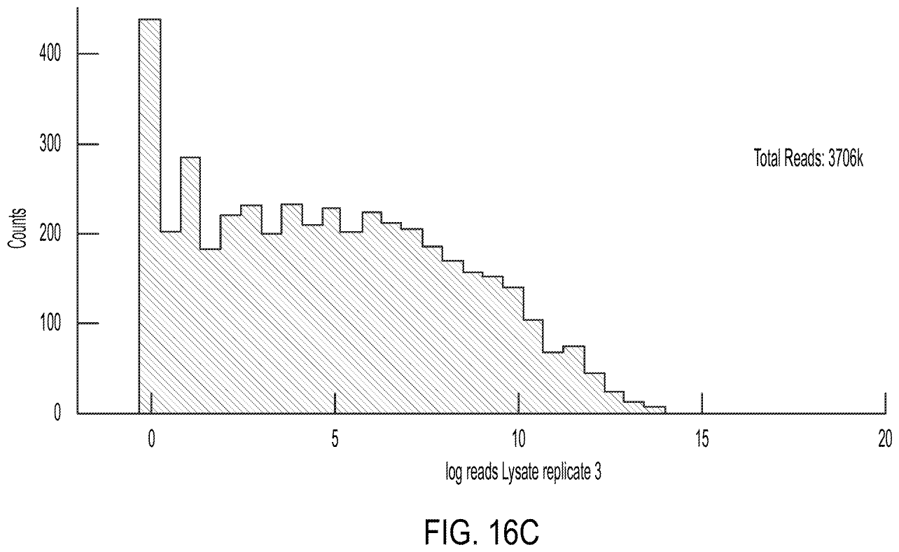

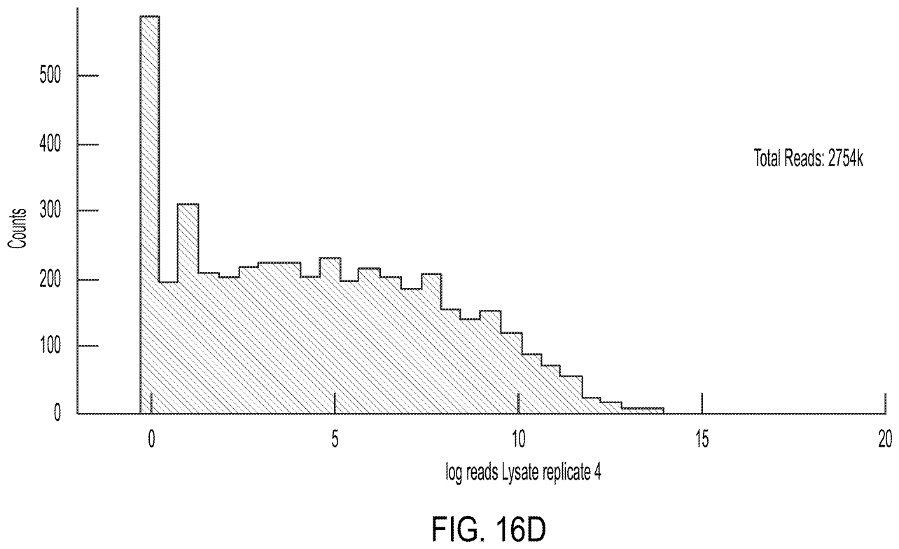

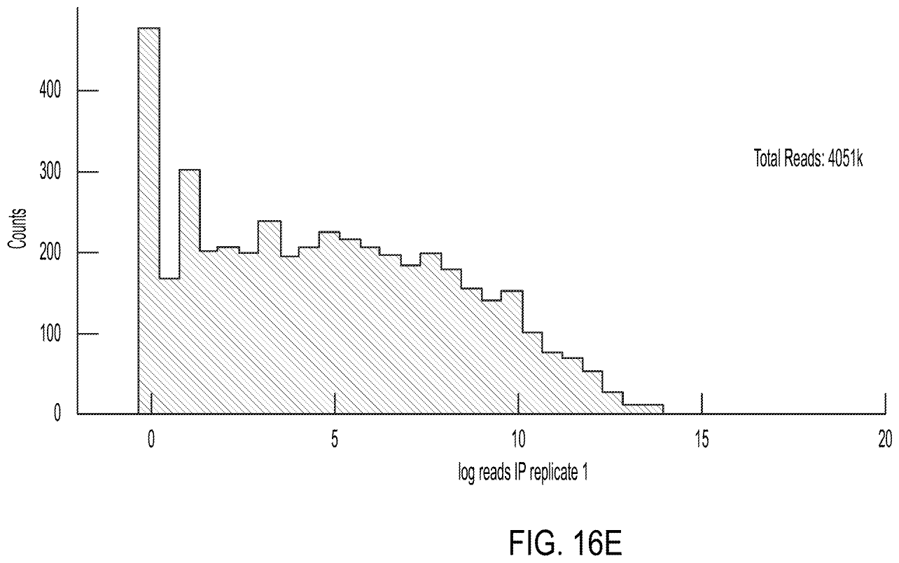

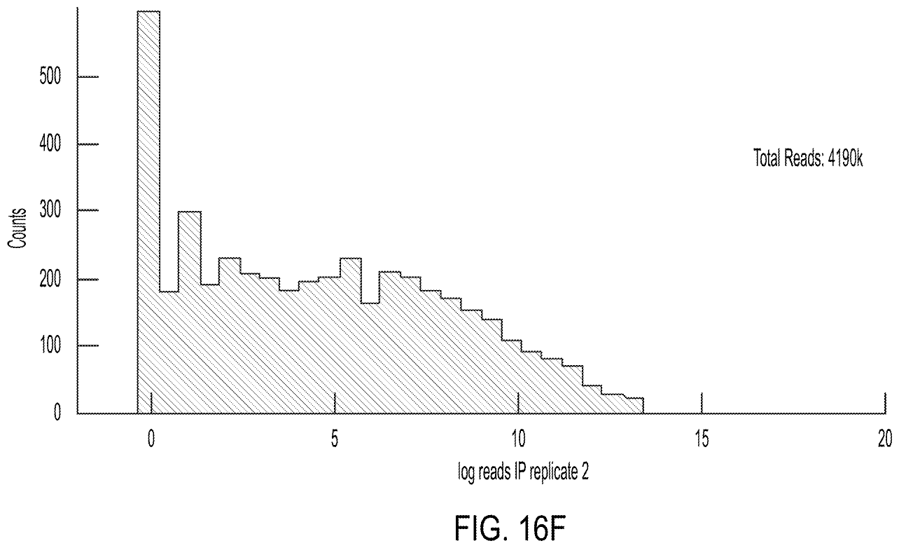

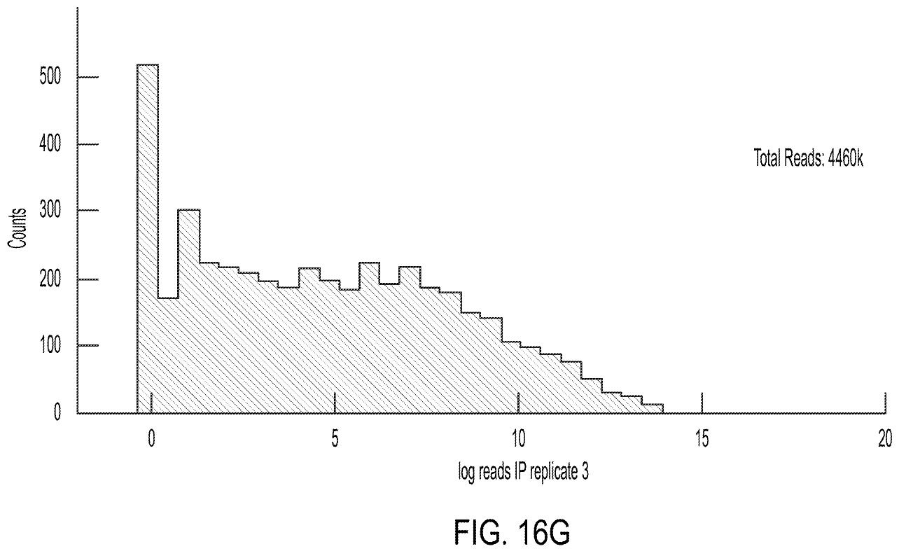

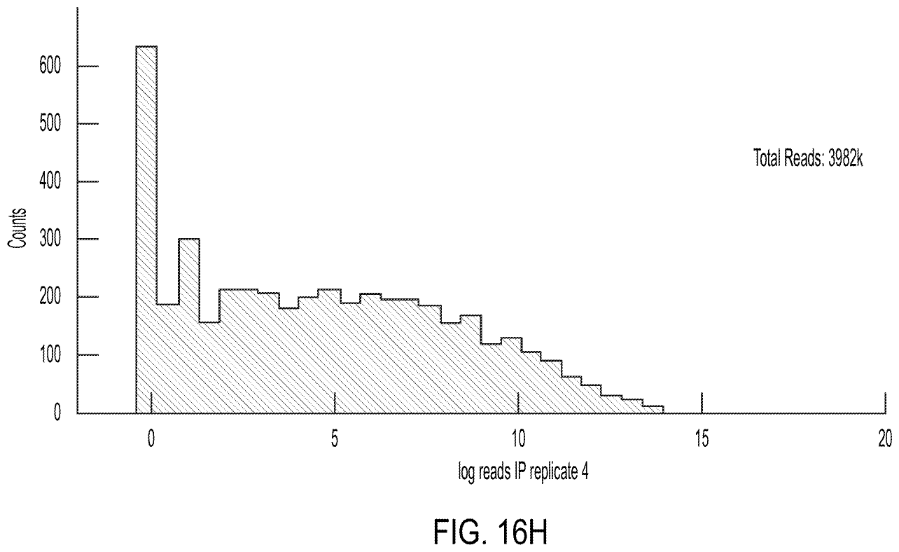

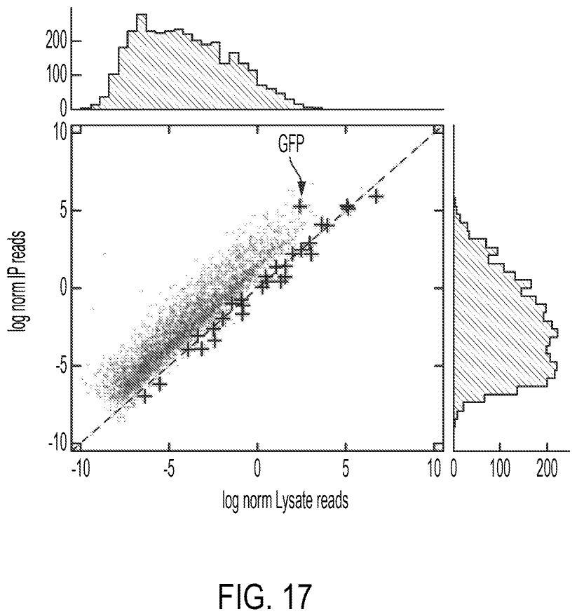

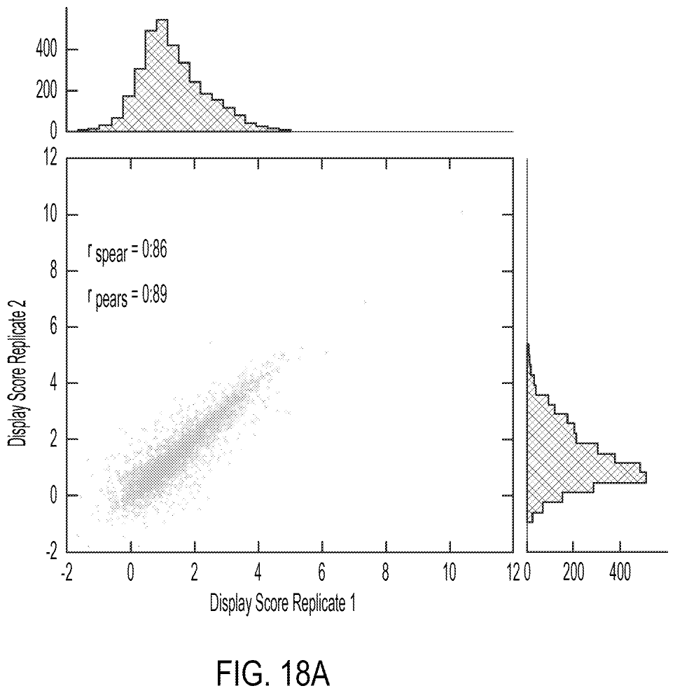

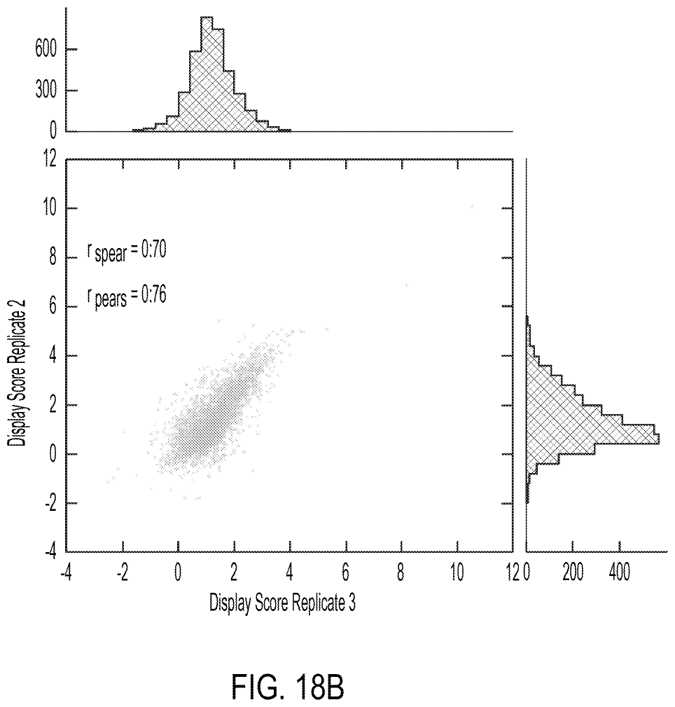

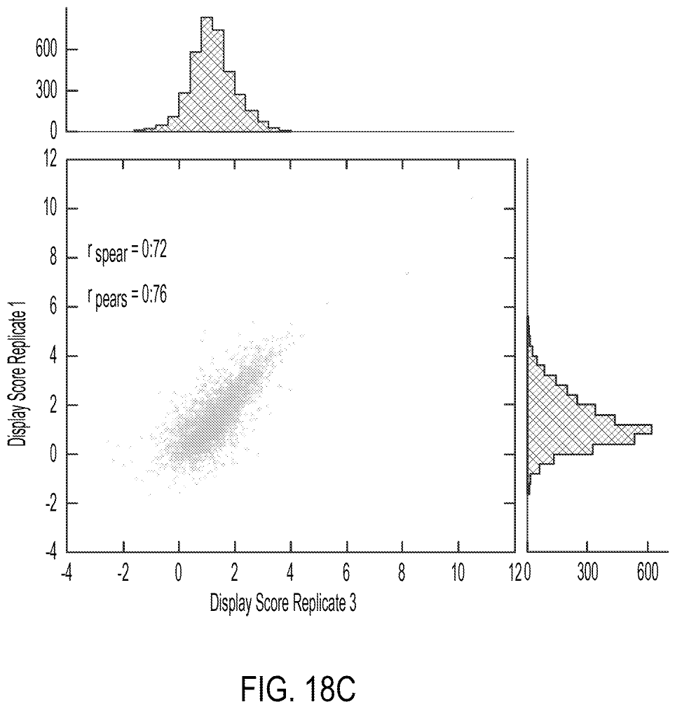

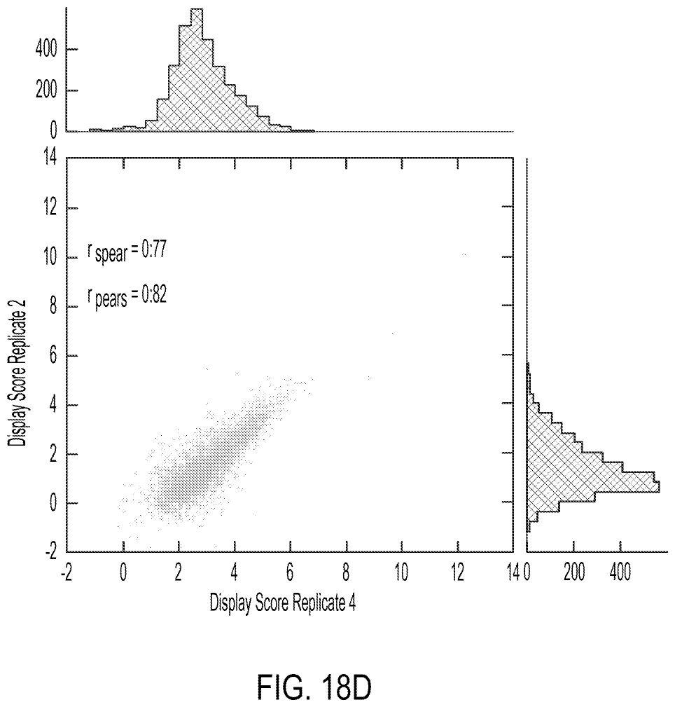

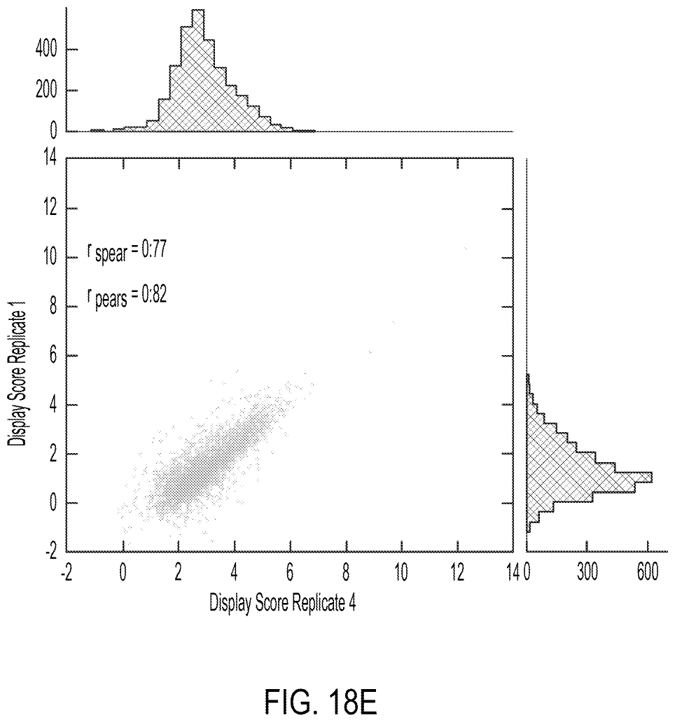

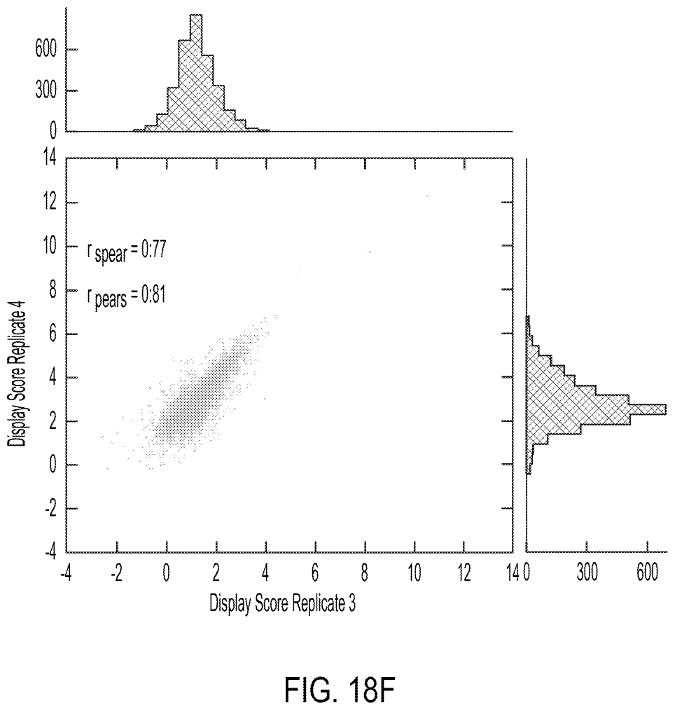

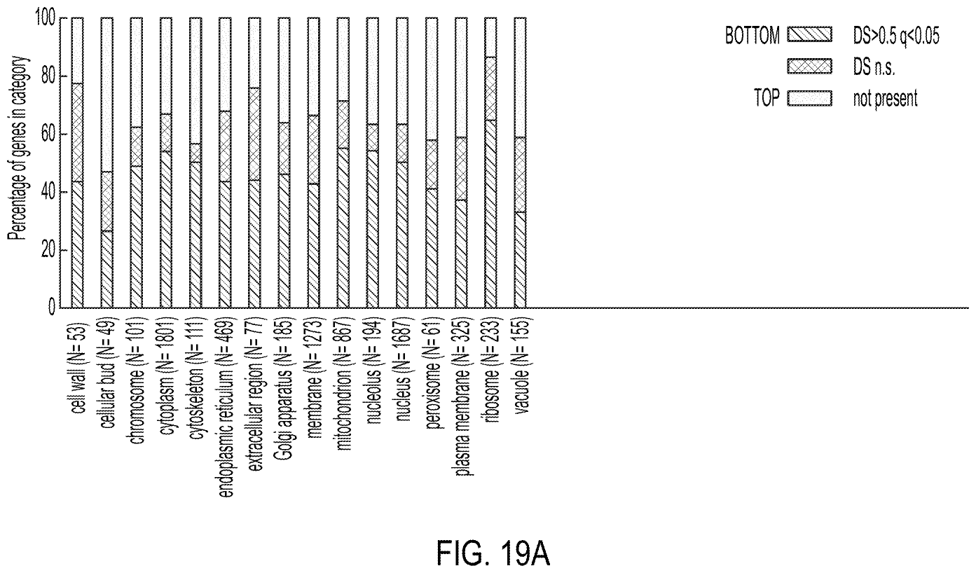

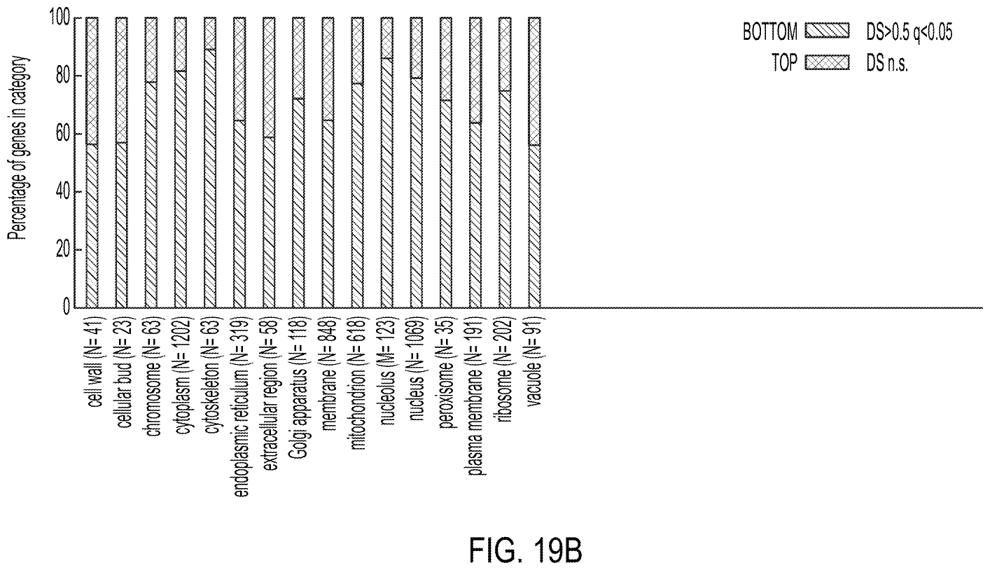

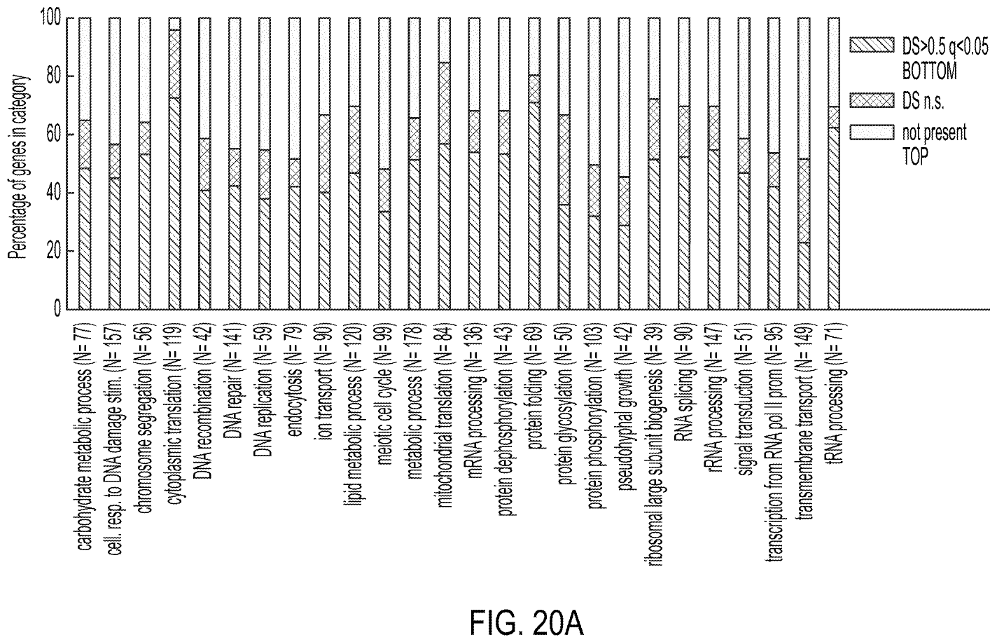

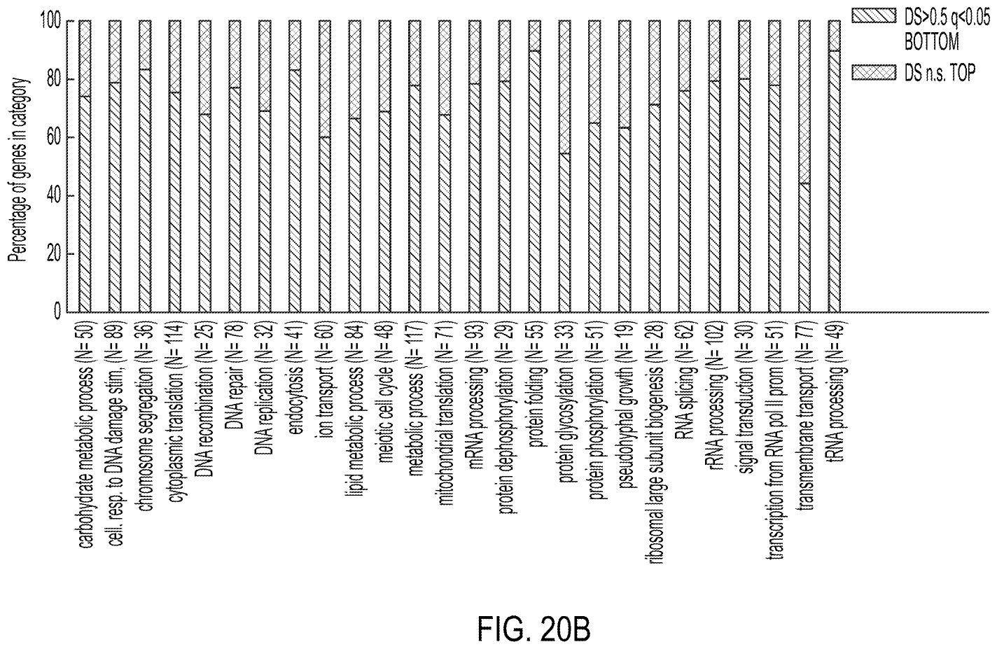

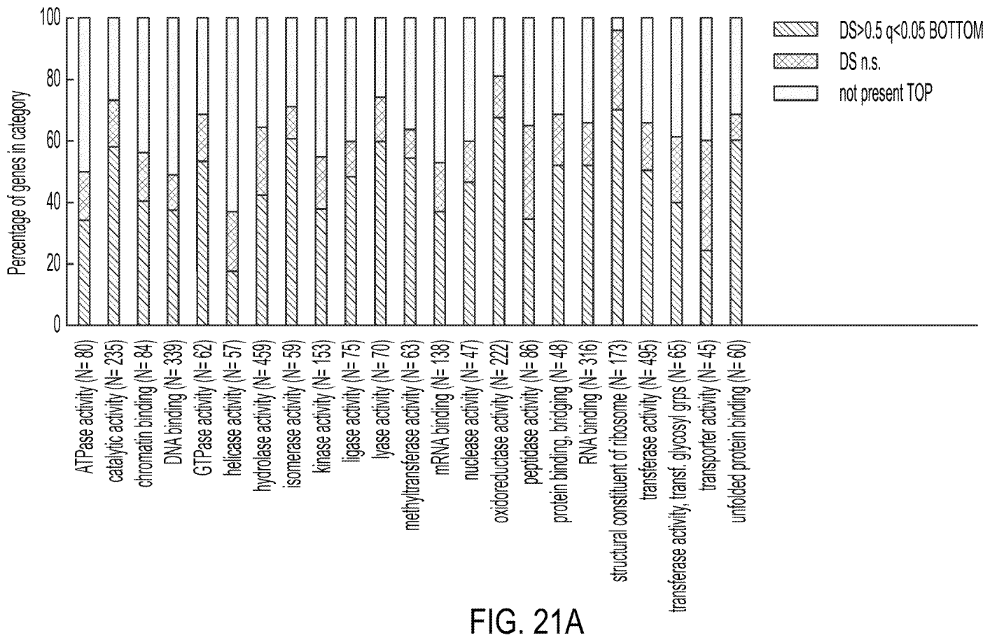

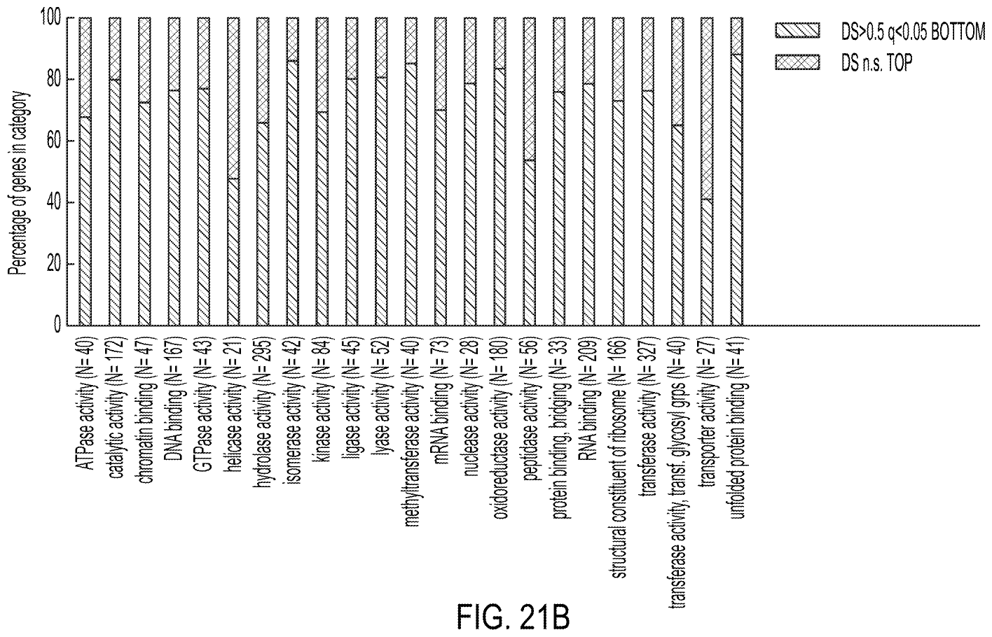

An in vivo display library of the yeast ORFeome was built for high throughput proteomic exploration. Starting from the plasmid ORFeome library⁵² encoding ˜4700 validated yeast proteins, the ORFs were pooled and introduced into an in vivo mRNA display backbone using the Gateway cloning system (see Methods). The resulting pooled library was transformed into the BY4742 S288c Matα strain. To estimate the overall ability of every protein to display its encoding mRNA effectively, the proteome was purified from library lysate utilizing a 6×HIS tag. As with all fusion libraries, the ability to capture interactions is limited by proper protein folding, the proper positioning of any functional domains as well as, for this approach, the ability of the capsid domain to bind the stem-loop efficiently. The 6×HIS tag was used for library construction in order to preserve other tags for future functional assays, when more specific tags would be needed for the purification of cellular complexes. Since the histidine tag purification has a relatively poor ability to enrich for bound RNA relative to other tags (FIG. 2C), this assay was expected to under-estimate display efficiency. Overall, the constructed yeast in vivo display library captured ˜3400 proteins, which were consistently present in either the lysate or the purified samples across four replicates (FIG. 2D). Each replicate was sequenced for <5M reads (FIGS. 16A-H) and calculated the Display Score of each ORF in the purified samples against the lysate, relative to the non-specific functional controls (FIG. 17). 73% of the ORFs captured in the assay exhibit a significant display enrichment score compared to the non-specific functional controls (average DES>0.5; Mann-Whitney U test, Benjamini-Hochberg corrected q-value<0.05, FIG. 2E). Display scores were reproducible across replicates (rₛₚₑₐᵣ=0.76-0.89, FIG. 2F, FIGS. 18A-F). Overall, yeast proteins that efficiently display their own mRNA span a wide range of biological processes, functions, and cellular compartments⁵³ (FIG. 2G, FIGS. 19-21).

In Vivo mRNA Display Retains Native Organellar Localization of the Proteome

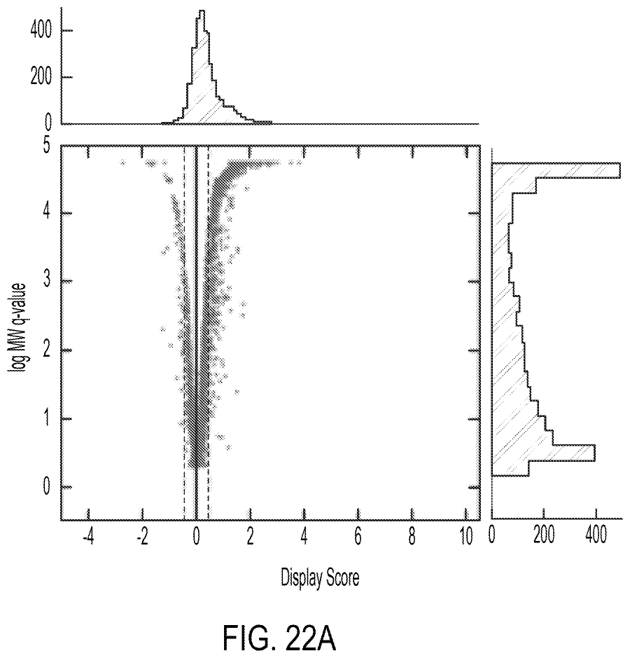

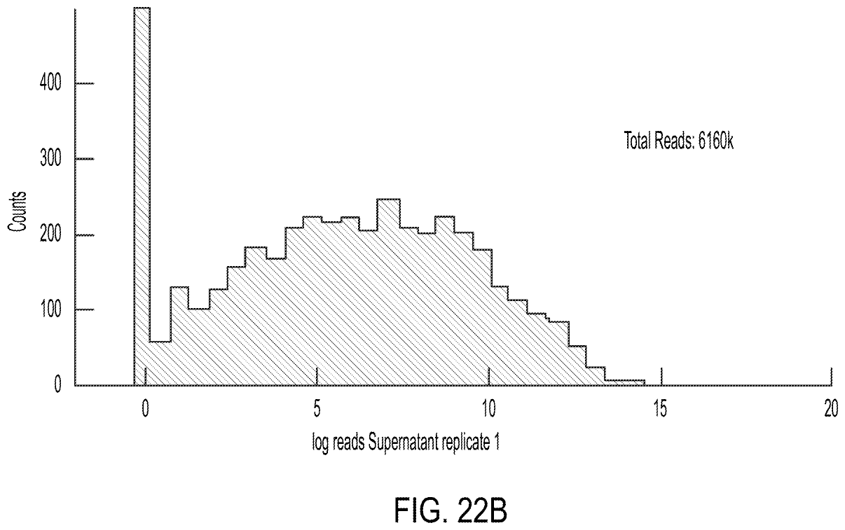

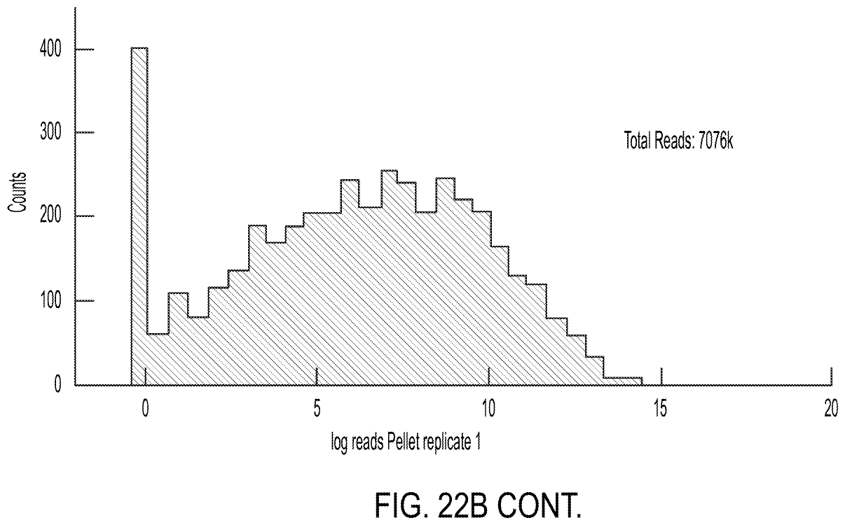

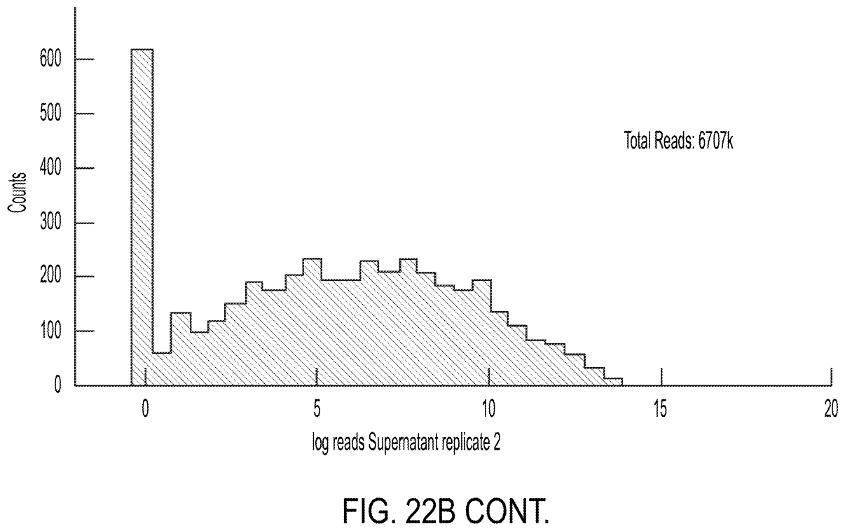

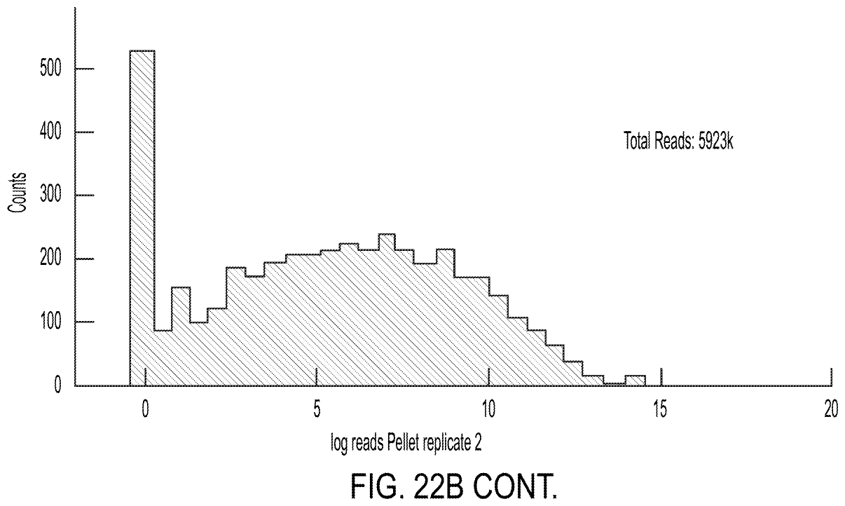

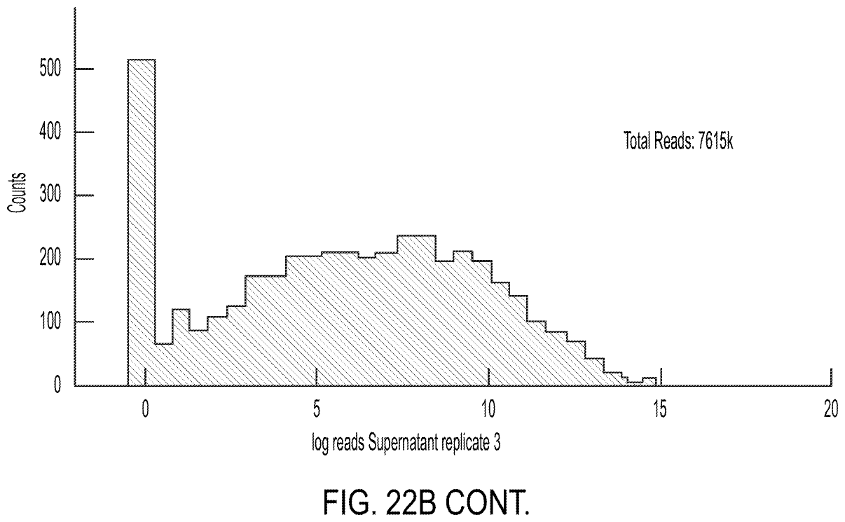

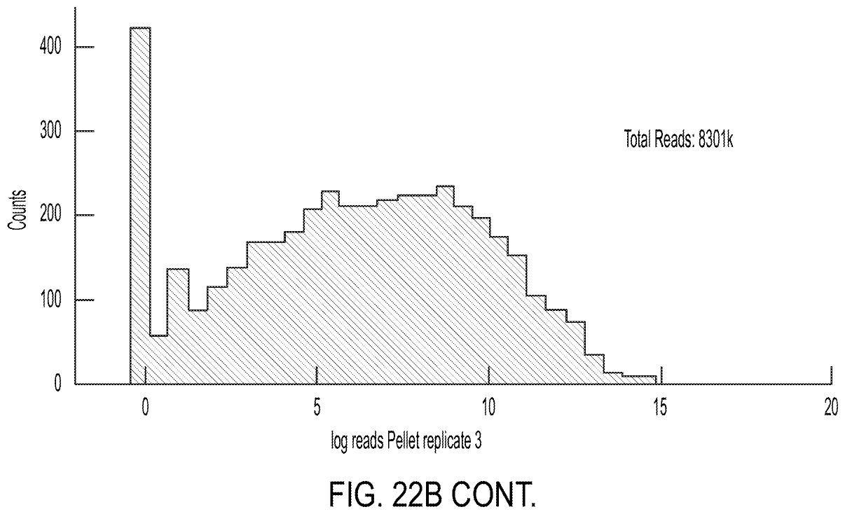

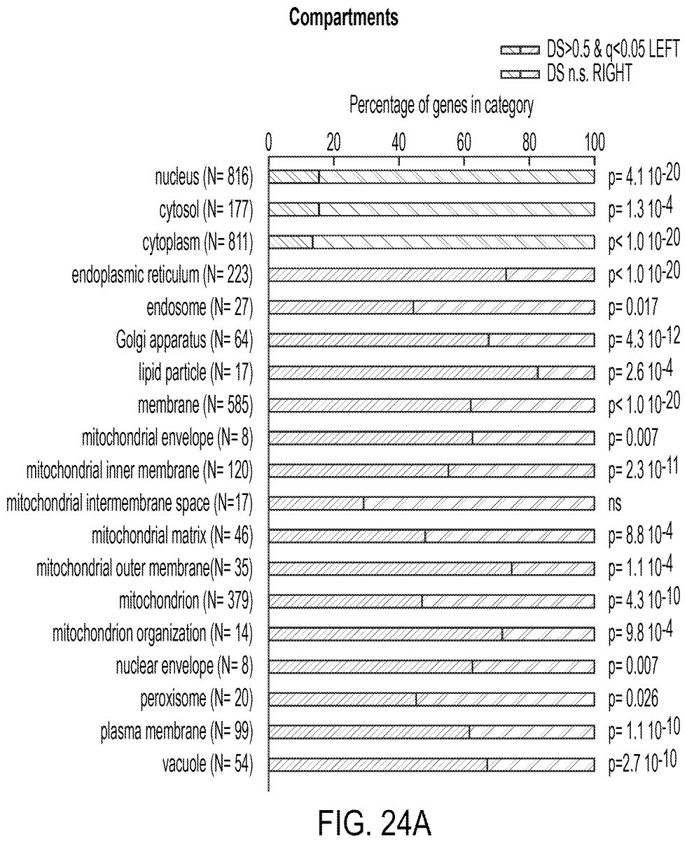

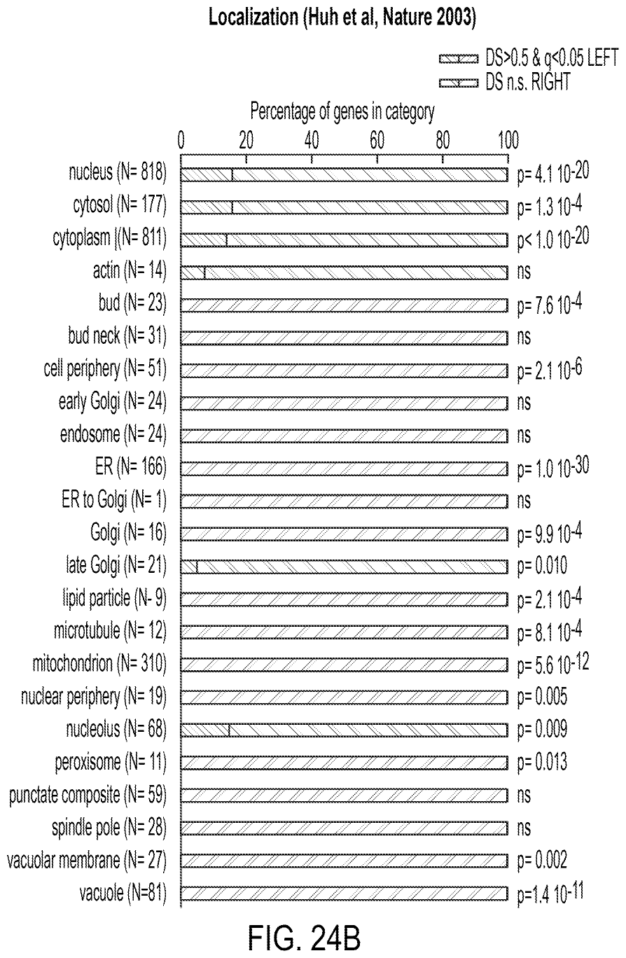

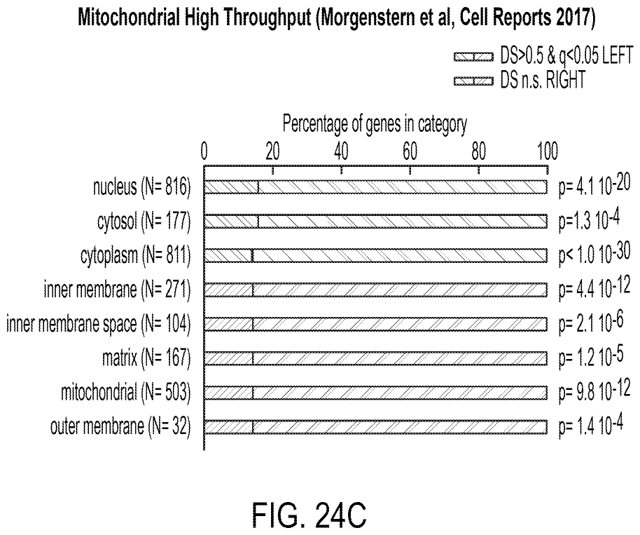

Do in vivo mRNA displayed proteins retain their native sub-cellular compartmentalization, despite their episomal over-expression, fusion with the capsid and association with their cognate mRNA? To test this, a subcellular fractionation experiment was performed in order to isolate proteins localized in specific cellular compartments. In particular, a crude mitochondrial purification^{54,55} was performed whereby induced in vivo displayed library spheroplasts were disrupted with a dounce homogenizer and a fraction was enriched by means of differential centrifugation in triplicate (see Methods). This crude mitochondrial fractionation is commonly used as it is fast and does not require large amounts of starting material, even though it is known to be enriched in proteins and membranes from other organelles. Thus, RNA from the supernatant was isolated and sequenced and the samples of the final centrifugation step were pelleted. A DS score was calculated comparing read frequencies for each mRNA displayed species present in the assay between the two fractions (see Methods, FIG. 22). For example, the mRNAs for mitochondrial outer membrane proteins TOM70 and porins POR1, POR2 are significantly enriched in the fraction compared to the non-specific controls (z Score=4.7, 4.3 and 5.9 respectively), as well as for inner membrane proteins COX7 and TIM23 (z Score=5.8, 6.0), and mitochondrial matrix proteins IDH1, and PUT1 (z Score=3.9, 4.1). On the other hand, in vivo display mRNAs for cytosolic proteins LEU2 (z Score=0.4), MPE1 (z Score=0.5), ASN2 (z Score=0.8) and SAM2 (z Score=0.8) are not significantly enriched in the organelle fraction (FIG. 3A). GO term enrichment analysis, showed that Display Scores are indicative of protein membership in the expected organelles (AUC=0.75; FIG. 23 FIG. 30). In general, proteins known to localize to the mitochondria⁵⁶⁻⁵⁸ were 3 times more likely to be significantly displayed in the pellet compared to cytosolic proteins (P<10⁻¹⁸, FIG. 3B, FIG. 24). The analysis revealed that proteins of the mitochondrial outer membrane (×4.7; P<10⁻⁸) and inner membrane proteins (×3.5; P<10⁻¹⁰) are all significantly enriched (FIGS. 3B, 24). Endoplasmic Reticulum, Golgi and lipid particle associated proteins were over 4-times more likely to be significantly displayed in the pellet. Also, as expected, proteins known to localize to the cytoplasm and nucleus were significantly depleted (P<10⁻²⁰ and P<10⁻¹⁹ respectively).

In Vivo mRNA Display Enables Accurate Discovery of In Vivo Protein-Protein Interactions

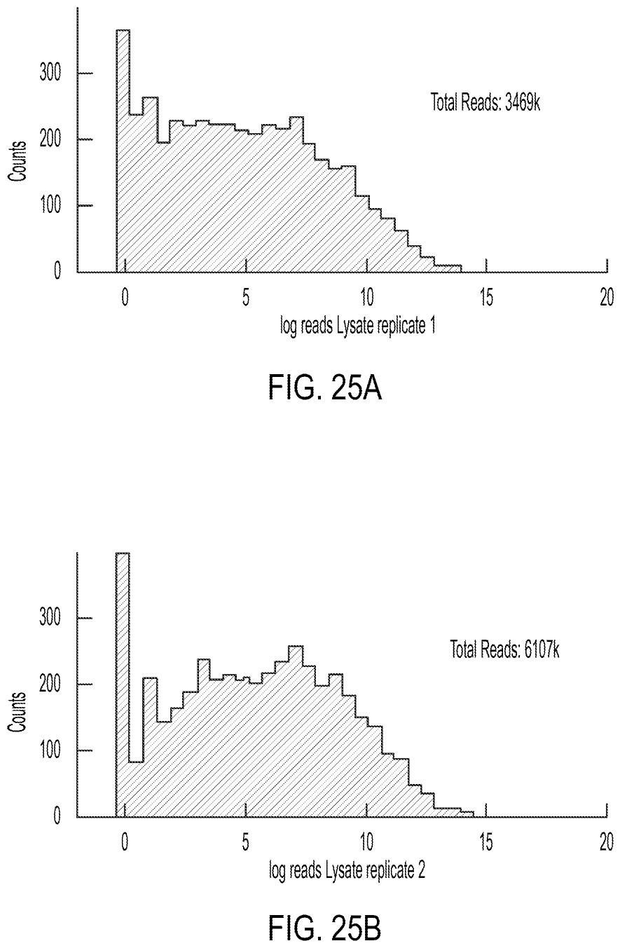

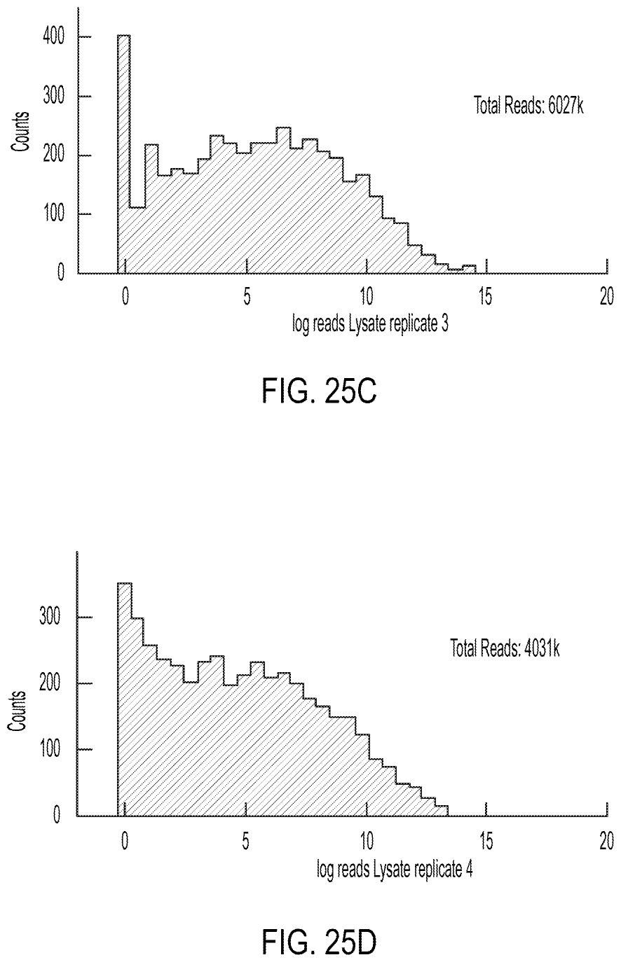

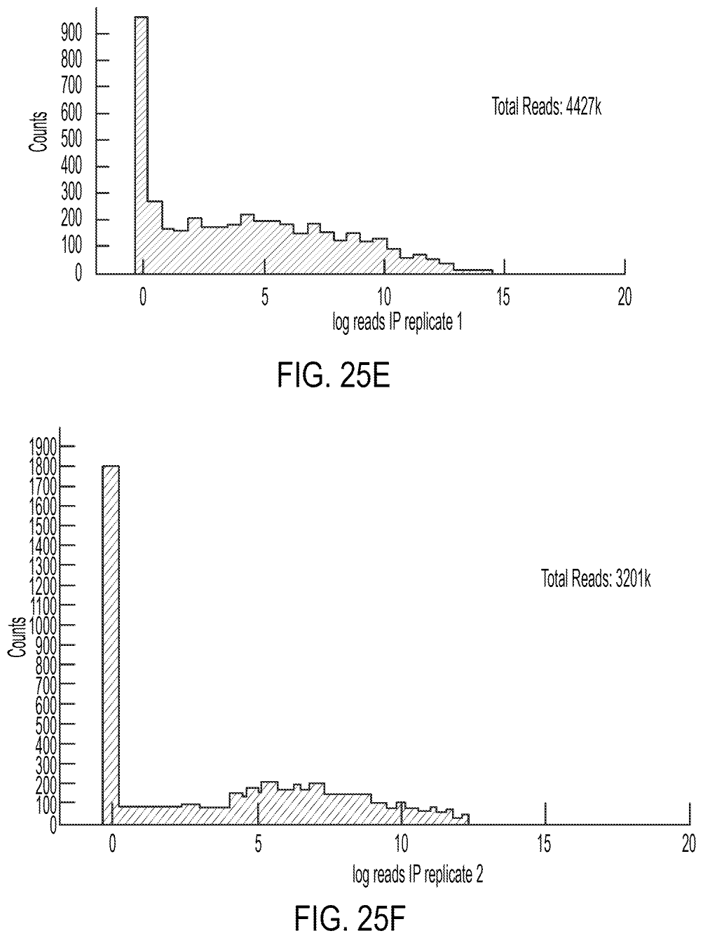

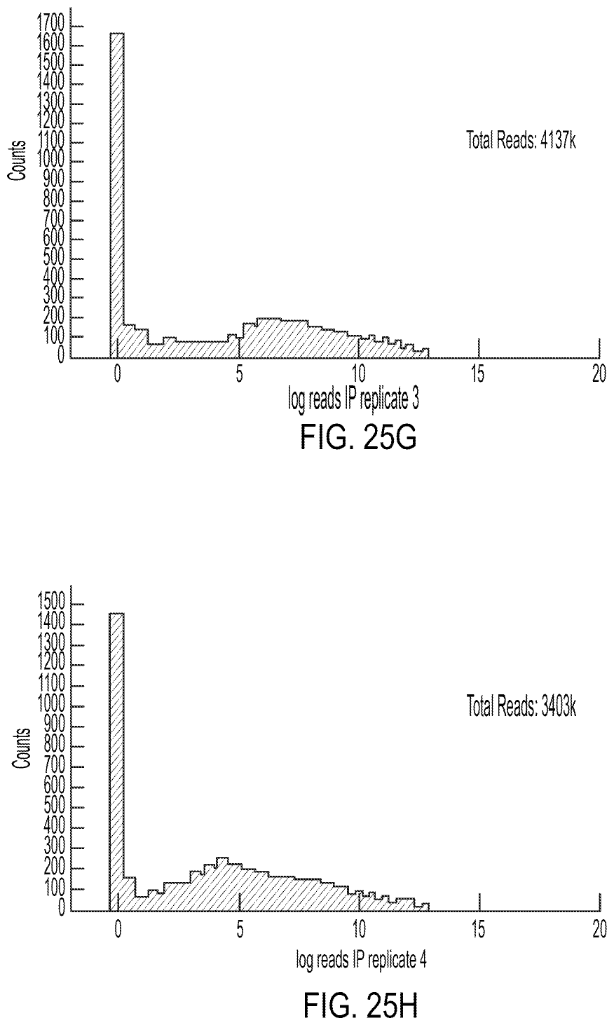

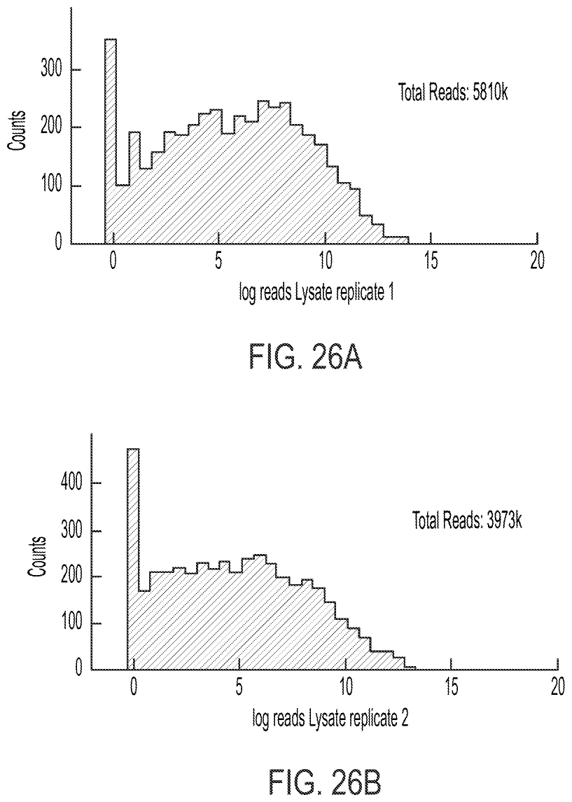

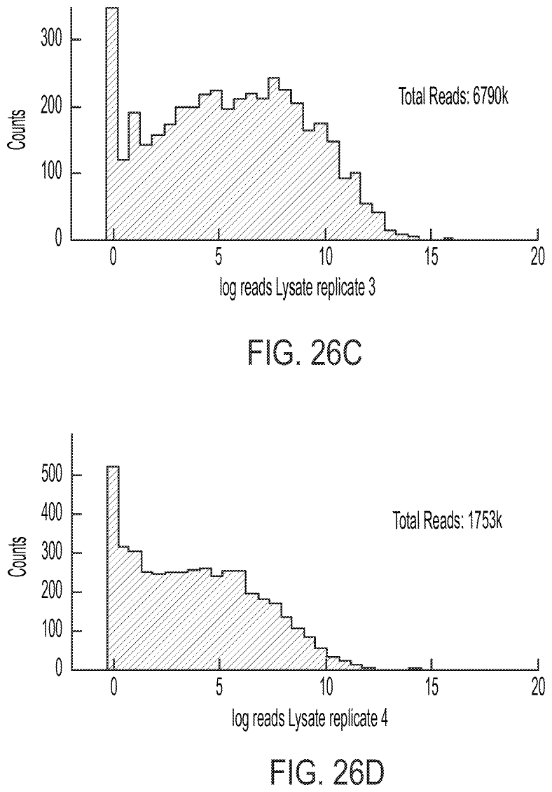

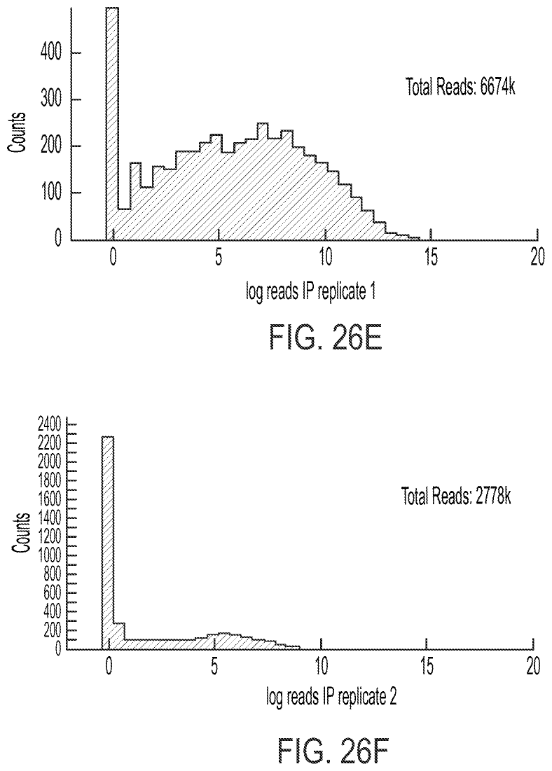

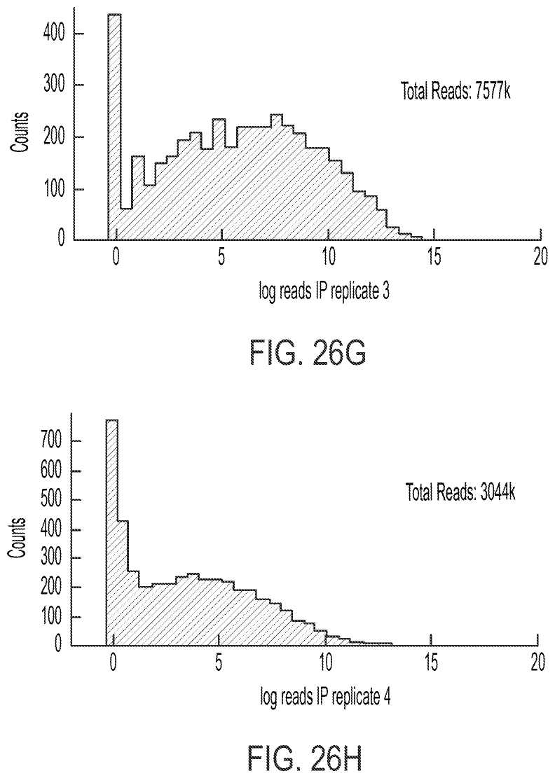

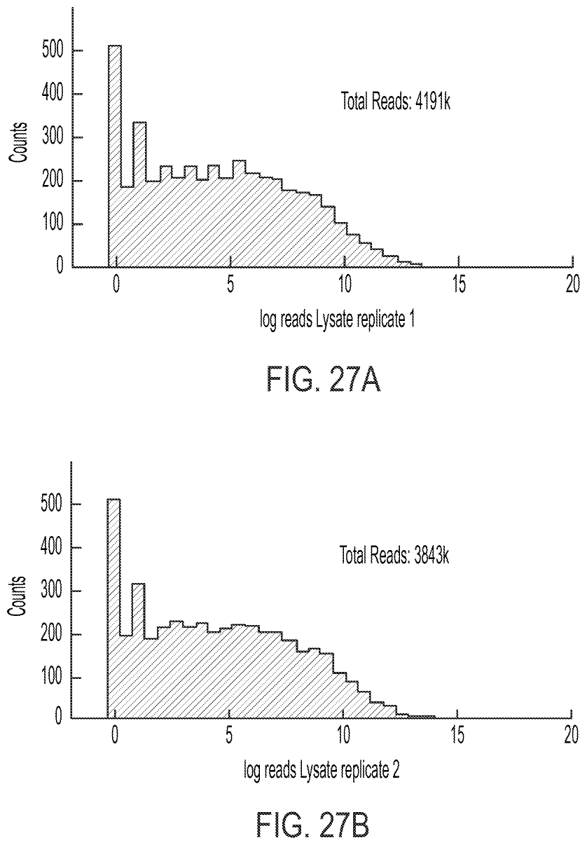

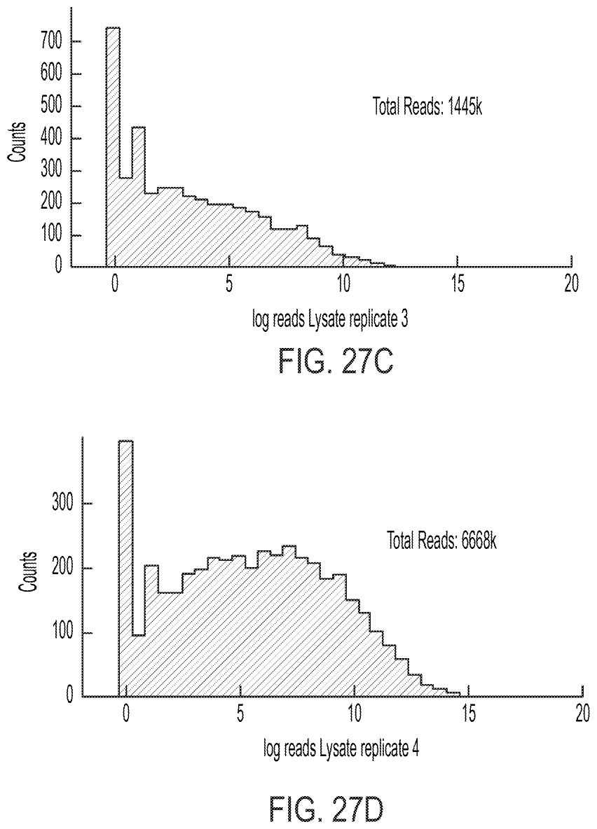

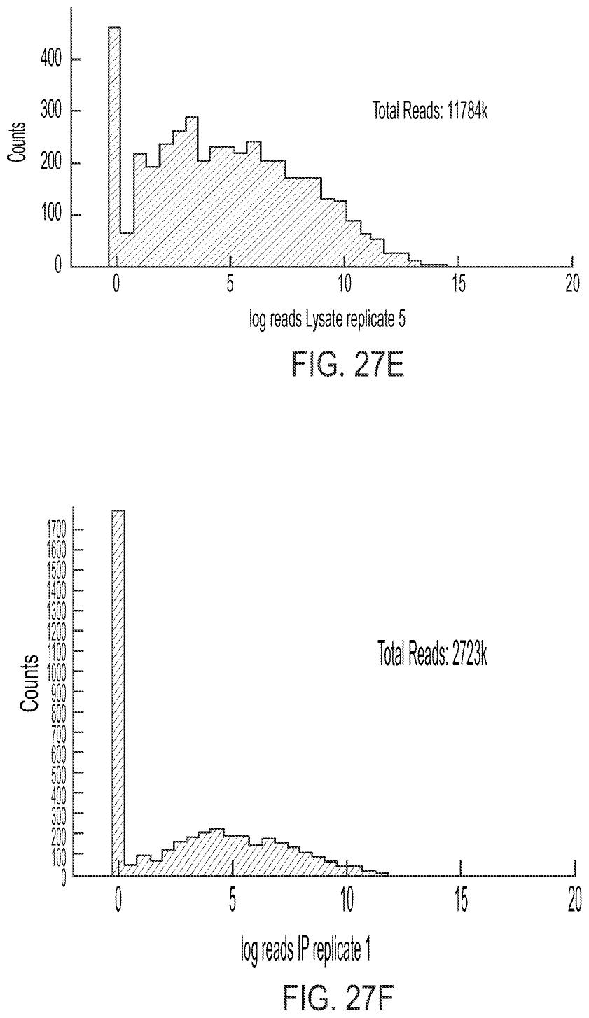

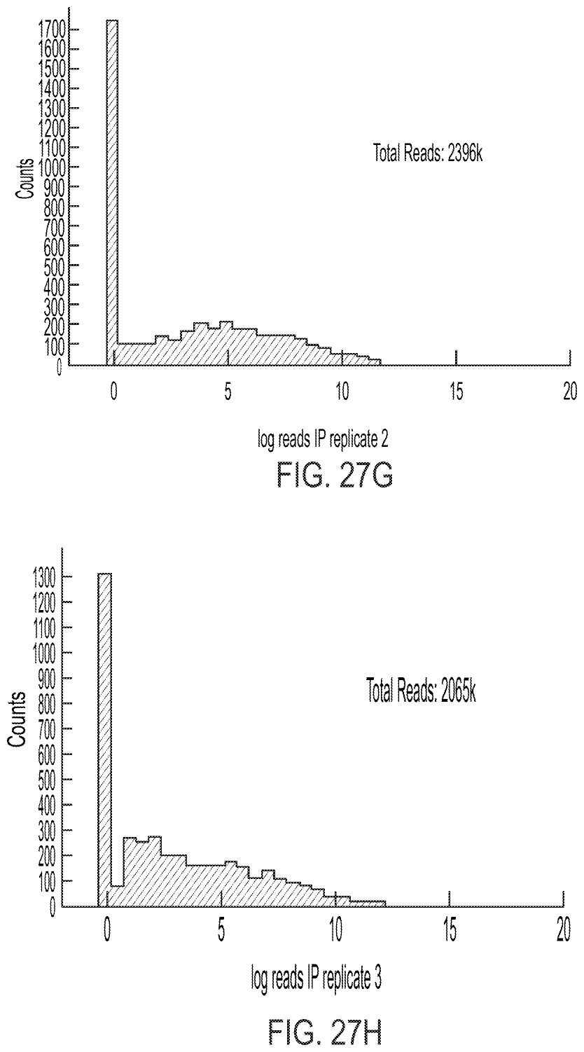

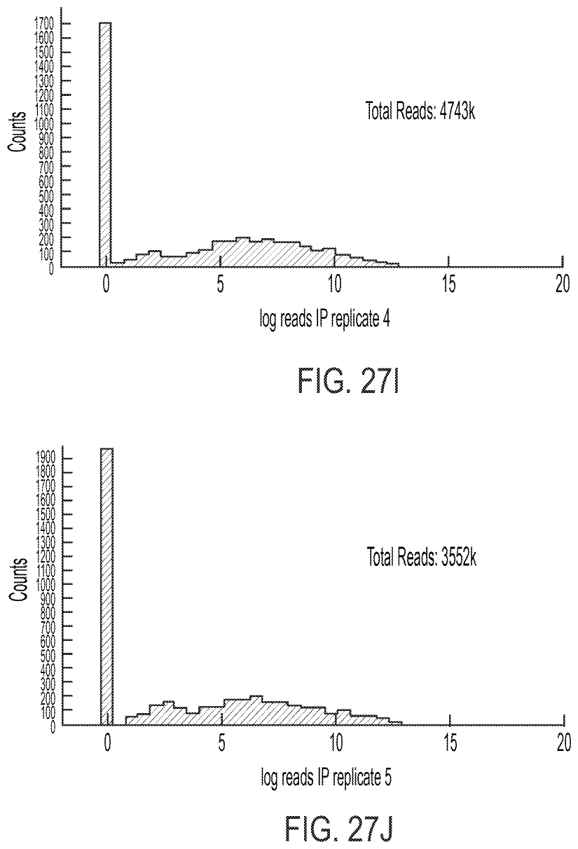

Mapping the network of protein-protein interactions (PPI) has been a central challenge of post-genome biology. In one embodiment, the subject matter disclosed herein relates to determining whether in vivo mRNA display can be used to efficiently identify the in vivo interaction partners of a protein of interest. Thus, libraries were generated for systematic PPI assays by mating the haploid in vivo display MATα library with a MATa strain expressing a protein bait of interest. The protein bait was fused with a C-terminal GFP epitope tag, enabling its efficient IP. After induction and homogenization, RNA reads from the lysate were compared to a sample purified using anti-GFP magnetic beads and calculated a corresponding Display Score. The interaction partners of two proteins were investigated: SAM2, a highly expressed S-adenosylmethionine synthetase⁵⁹, and ARC40, a member of the Arp2/3 complex that is an actin nucleation center playing a critical role in the motility and integrity of actin patches^{60,61}. Two libraries were generated for each of the SAM2- and ARC40-GFP baits, one with the fusion protein integrated into the genome and driven by the native promoter⁵⁷, and another episomally expressed and inducible. In addition, two control libraries were generated containing either an inducible GFP, not fused to any other peptides, or a null library containing no bait. Each of the described libraries were tested in duplicate (FIGS. 25-27).

For a given bait (SAM2 or ARC40), a library protein was considered to be a PPI hit if the mRNA of the corresponding ORF was enriched in the corresponding samples (average DS>2; q-value<0.001; FIG. 3C-G) compared to the lysate but not enriched in the control samples (q-value>0.05). For SAM2, two hits were found: SAM2 itself (DES=4.8, q-value=6×10⁻⁴) and its paralog SAM1 (DS=4.4, q-value=6×10⁻⁴; FIG. 3C, 3F). Indeed, SAM2 has been reported to interact with its paralog in traditional affinity capture-MS studies^{21,22} and it has also been predicted to interact with itself by Y2H⁶. On the other hand, the hits for ARC40 are members of the same complex⁶¹: ARC19 (DS=3.9, q-value=1×10⁻⁵), ARC35 (DS=3.7, q-value=1.1×10⁻⁵) and ARC18 (DS=3.3, q-value=1.3×10⁻⁵; FIG. 3D, 3G). ARC40 forms a seven subunit complex along with ARC19, ARC35, ARC18, ARC15, ARP2 and ARP3⁶¹. ARP2 is only moderately enriched in our assay (DS=0.55, q-value=0.006), while ARP3 is not enriched in the purified ARC40 samples. ARC15 is not present in the library and, therefore, could not be assessed.

Affinity capture was performed followed by LC-MS/MS to validate the results using samples processed identically to the in vivo display assays. It was confirmed that SAM1 was co-purified with SAM2, while ARC40 samples were enriched in ARP2/3 complex subunits (FIG. 3H). Additionally, actin related proteins MYO3, MYO5, and ACT1 were enriched in the ARC40 samples. MYO3 was not a member of the pooled library, while MYO5 was not included in the yeast ORFeome set. Mass spectrometry cannot discriminate between self-interaction and presence as a bait and, hence, the identified targets SAM2 and ARC40 (FIG. 3H) are due to the purified bait itself. On the other hand, in vivo mRNA display is able to capture such self-interactions as demonstrated by the enrichment of SAM2 reads in the SAM2 purified samples.

The lack of strong enrichment for the known ARC40 interactors ARP2 and ARP3 may be due to multiple factors. These include the inability of the MCP fusions to fold properly, or to bind their respective mRNAs efficiently, the interference of the fused domains with the interaction under study, or even library construction biases. In order to probe the sensitivity of in vivo mRNA display further, a low throughput display experiment was designed that included all the possible targets of ARC40 from the mass spectrometry assay. The respective ORFs were cloned into the construct one at a time and their sequences were validated. In addition to ARC35, ARC18 and ARC19, ARP2 (DS=1.8, q-value=0.002) and MYO3 (DS=1.9, q-value=0.0015) were significantly enriched when ARC40 is purified, while they are not enriched in SAM2 samples (FIG. 3I). On the other hand, ARC15, ARP3 and ACT1 are not enriched, showcasing possible limitations of the approach. While ARC15 was not present in the high throughput library, ARP3 and ACT1 did not significantly display their mRNA in the whole library purification assay which explains the lack of enrichment in the co-purification assay

The MS2-MCP interaction enables stable non-covalent linking of proteins to their encoding mRNAs in vivo. This feature can be exploited to convert a variety of standard proteomics-based assays to a sequencing readout. The in vivo mRNA displayed proteins maintain their organellar distributions in a manner that can be utilized for sequencing based protein cartography. In vivo mRNA display can be used for high specificity detection of in vivo protein-protein interactions. However, our first demonstration of this technology has some limitations. In some embodiments, the display protein can be decoupled from the displayed mRNA utilizing a library of same length bar-codes. In some embodiments, the technology employs an automated ORF by ORF library construction (versus the simple pooled approach).

Overall, in vivo mRNA display enables high throughput proteomics, leveraging the ease, cost, and capacity for massive parallelization of NGS. While a medium throughput mass spectrometry experiment can cost over $1000, the same samples can be processed for ˜ 1/10th of the cost with in vivo mRNA display. As with all display technologies, such as Y2H and phage display, this approach depends on library construction which requires some initial labor and cost but this initial investment would pay off in the longer-term benefits of this resource for diverse applications across the community. Moreover, in vivo mRNA display interrogates proteins in their native cellular context, including post-translational modifications, the presence of co-factors and subcellular localization, making it compatible with affinity capture assays, which are the gold standard for proteomics. NGS has revolutionized genomics, in vivo mRNA display has the potential of similarly improving the throughput, labor, and cost of a variety of proteomics applications.

Protein functional studies are critical in studying basic biology, but also in better understanding the molecular etiology of disease and development of novel therapeutics. As the MS2 tagging system has already been utilized in many different cellular contexts, in vivo mRNA display can be a powerful tool for proteomic studies in mammalian systems. Furthermore, much as other display technologies such as phage-display have enabled in vitro protein optimization in industrial and biomedical applications^{62,63}, in vivo mRNA display will enable similar optimization of peptides and proteins for therapeutic benefit within physiologically relevant contexts in vivo.

### References for Example 1

1. Altelaar, A. F. M., Munoz, J. & Heck, A. J. R. Next-generation proteomics: towards an integrative view of proteome dynamics. Nat. Rev. Genet. 14, 35-48 (2013).
2. Snider, J. et al. Fundamentals of protein interaction network mapping. Mol. Syst. Biol. 11, 848 (2015).
3. Morris, J. H. et al. Affinity purification-mass spectrometry and network analysis to understand protein-protein interactions. Nat. Protoc. 9, 2539-2554 (2014).
4. Havugimana, P. C. et al. A Census of Human Soluble Protein Complexes. Cell 150, 1068-1081 (2012).
5. Fields, S. & Song, O. A novel genetic system to detect protein-protein interactions. Nature 340, 245-246 (1989).
6. Yu, H. et al. High-quality binary protein interaction map of the yeast interactome network. Science 322, 104-110 (2008).
7. Snider, J. et al. Detecting interactions with membrane proteins using a membrane two-hybrid assay in yeast. Nat. Protoc. 5, 1281-1293 (2010).
8. Kenworthy, A. K. Imaging protein-protein interactions using fluorescence resonance energy transfer microscopy. Methods San Diego Calif. 24, 289-296 (2001).
9. Remy, I. & Michnick, S. W. Clonal selection and in vivo quantitation of protein interactions with protein-fragment complementation assays. Proc. Natl. Acad. Sci. 96, 5394-5399 (1999).
10. Tarassov, K. et al. An in Vivo Map of the Yeast Protein Interactome. Science 320, 1465-1470 (2008).
11. Weill, U. et al. Genome-wide SWAp-Tag yeast libraries for proteome exploration. Nat. Methods 15, 617-622 (2018).
12. Branon, T. C. et al. Efficient proximity labeling in living cells and organisms with TurboID. Nat. Biotechnol. 36, 880-887 (2018).
13. Roux, K. J., Kim, D. I., Raida, M. & Burke, B. A promiscuous biotin ligase fusion protein identifies proximal and interacting proteins in mammalian cells. J. Cell Biol. 196, 801-810 (2012).
14. Fernández-Suárez, M., Chen, T. S. & Ting, A. Y. Protein-protein interaction detection in vitro and in cells by proximity biotinylation. J. Am. Chem. Soc. 130, 9251-9253 (2008).
15. Rhee, H.-W. et al. Proteomic mapping of mitochondria in living cells via spatially restricted enzymatic tagging. Science 339, 1328-1331 (2013).
16. Hung, V. et al. Proteomic mapping of cytosol-facing outer mitochondrial and ER membranes in living human cells by proximity biotinylation. eLife 6, e24463 (2017).
17. Lobingier, B. T. et al. An Approach to Spatiotemporally Resolve Protein Interaction Networks in Living Cells. Cell 169, 350-360.e12 (2017).
18. Hein, M. Y. et al. A Human Interactome in Three Quantitative Dimensions Organized by Stoichiometries and Abundances. Cell 163, 712-723 (2015).
19. Huttlin, E. L. et al. Architecture of the human interactome defines protein communities and disease networks. Nature 545, 505-509 (2017).
20. Wan, C. et al. Panorama of ancient metazoan macromolecular complexes. Nature 525, 339-344 (2015).
21. Gavin, A.-C. et al. Proteome survey reveals modularity of the yeast cell machinery. Nature 440, 631-636 (2006).
22. Krogan, N. J. et al. Global landscape of protein complexes in the yeast Saccharomyces cerevisiae. Nature 440, 637-643 (2006).
23. Rolland, T. et al. A Proteome-Scale Map of the Human Interactome Network. Cell 159, 1212-1226 (2014).
24. Luck, K., Sheynkman, G. M., Zhang, I. & Vidal, M. Proteome-Scale Human Interactomics. Trends Biochem. Sci. 42, 342-354 (2017).
25. Vidal, M., Cusick, M. E. & Barabási, A.-L. Interactome Networks and Human Disease. Cell 144, 986-998 (2011).
26. Menche, J. et al. Uncovering disease-disease relationships through the incomplete interactome. Science 347, 1257601-1257601 (2015).
27. Kovács, I. A. et al. Network-based prediction of protein interactions. Nat. Commun. 10, 1240 (2019).
28. Lieberman-Aiden, E. et al. Comprehensive mapping of long-range interactions reveals folding principles of the human genome. Science 326, 289-293 (2009).
29. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
30. Krueger, F., Kreck, B., Franke, A. & Andrews, S. R. DNA methylome analysis using short bisulfite sequencing data. Nat. Methods 9, 145-151 (2012).
31. Sidoli, S., Kulej, K. & Garcia, B. A. Why proteomics is not the new genomics and the future of mass spectrometry in cell biology. J. Cell Biol. 216, 21-24 (2017).
32. Low, T. Y., Mohtar, M. A., Ang, M. Y. & Jamal, R. Connecting Proteomics to Next-Generation Sequencing: Proteogenomics and Its Current Applications in Biology. PROTEOMICS 19, 1800235 (2019).
33. Smith, G. P. Filamentous fusion phage: novel expression vectors that display cloned antigens on the virion surface. Science 228, 1315-1317 (1985).
34. McCafferty, J., Griffiths, A. D., Winter, G. & Chiswell, D. J. Phage antibodies: filamentous phage displaying antibody variable domains. Nature 348, 552-554 (1990).
35. Smith, G. P. & Petrenko, V. A. Phage Display. Chem. Rev. 97, 391-410 (1997).
36. Sidhu, S. S. & Koide, S. Phage display for engineering and analyzing protein interaction interfaces. Curr. Opin. Struct. Biol. 17, 481-487 (2007).
37. Nemoto, N., Miyamoto-Sato, E., Husimi, Y. & Yanagawa, H. In vitro virus: bonding of mRNA bearing puromycin at the 3′-terminal end to the C-terminal end of its encoded protein on the ribosome in vitro. FEBS Lett. 414, 405-408 (1997).
38. Roberts, R. W. & Szostak, J. W. RNA-peptide fusions for the in vitro selection of peptides and proteins. Proc. Natl. Acad. Sci. U.S.A. 94, 12297-12302 (1997).
39. Hanes, J. & Plückthun, A. In vitro selection and evolution of functional proteins by using ribosome display. Proc. Natl. Acad. Sci. U.S.A. 94, 4937-4942 (1997).
40. Boder, E. T. & Wittrup, K. D. Yeast surface display for screening combinatorial polypeptide libraries. Nat. Biotechnol. 15, 553-557 (1997).
41. Gu, L. et al. Multiplex single-molecule interaction profiling of DNA-barcoded proteins. Nature 515, 554-557 (2014).
42. Larman, H. B., Liang, A. C., Elledge, S. J. & Zhu, J. Discovery of protein interactions using parallel analysis of translated ORFs (PLATO). Nat. Protoc. 9, 90-103 (2014).
43. Younger, D., Berger, S., Baker, D. & Klavins, E. High-throughput characterization of protein-protein interactions by reprogramming yeast mating. Proc. Natl. Acad. Sci. 114, 12166-12171 (2017).
44. Trigg, S. A. et al. CrY2H-seq: a massively multiplexed assay for deep-coverage interactome mapping. Nat. Methods 14, 819-825 (2017).
45. Peng, X. et al. Affinity capture of polyribosomes followed by RNAseq (ACAPseq), a discovery platform for protein-protein interactions. eLife 7, e40982 (2018).
46. Layton, C. J., McMahon, P. L. & Greenleaf, W. J. Large-Scale, Quantitative Protein Assays on a High-Throughput DNA Sequencing Chip. Mol. Cell 73, 1075-1082.e4 (2019).
47. Johansson, H. E., Liljas, L. & Uhlenbeck, O. C. RNA Recognition by the MS2 Phage Coat Protein. Semin. Virol. 8, 176-185 (1997).
48. Peabody, D. S. The RNA binding site of bacteriophage MS2 coat protein. EMBO J. 12, 595-600 (1993).
49. Tyagi, S. Imaging intracellular RNA distribution and dynamics in living cells. Nat. Methods 6, 331-338 (2009).
50. Bertrand, E. et al. Localization of ASH1 mRNA particles in living yeast. Mol. Cell 2, 437-445 (1998).
51. Hocine, S., Raymond, P., Zenklusen, D., Chao, J. A. & Singer, R. H. Single-molecule analysis of gene expression using two-color RNA labeling in live yeast. Nat. Methods 10, 119-121 (2013).
52. Gelperin, D. M. et al. Biochemical and genetic analysis of the yeast proteome with a movable ORF collection. Genes Dev. 19, 2816-2826 (2005).
53. The Gene Ontology Consortium. The Gene Ontology Resource: 20 years and still GOing strong. Nucleic Acids Res. 47, D330-D338 (2019).
54. Liao, P.-C., Boldogh, I. R., Siegmund, S. E., Freyberg, Z. & Pon, L. A. Isolation of mitochondria from Saccharomyces cerevisiae using magnetic bead affinity purification. PLOS ONE 13, e0196632 (2018).
55. Daum, G., Böhni, P. C. & Schatz, G. Import of proteins into mitochondria. Cytochrome b2 and cytochrome c peroxidase are located in the intermembrane space of yeast mitochondria. J. Biol. Chem. 257, 13028-13033 (1982).
56. Ashburner, M. et al. Gene ontology: tool for the unification of biology. The Gene Ontology Consortium. Nat. Genet. 25, 25-29 (2000).
57. Huh, W.-K. et al. Global analysis of protein localization in budding yeast. Nature 425, 686-691 (2003).
58. Morgenstern, M. et al. Definition of a High-Confidence Mitochondrial Proteome at Quantitative Scale. Cell Rep. 19, 2836-2852 (2017).
59. Thomas, D., Rothstein, R., Rosenberg, N. & Surdin-Kerjan, Y. SAM2 encodes the second methionine S-adenosyl transferase in Saccharomyces cerevisiae: physiology and regulation of both enzymes. Mol. Cell. Biol. 8, 5132-5139 (1988).
60. Winter, D. C., Choe, E. Y. & Li, R. Genetic dissection of the budding yeast Arp2/3 complex: a comparison of the in vivo and structural roles of individual subunits. Proc. Natl. Acad. Sci. U.S.A. 96, 7288-7293 (1999).
61. Robinson, R. C. et al. Crystal structure of Arp2/3 complex. Science 294, 1679-1684 (2001).
62. Hoogenboom, H. R. Overview of Antibody Phage-Display Technology and Its Applications. in Antibody Phage Display vol. 178 001-037 (Humana Press, 2001).
63. Clackson, T., Hoogenboom, H. R., Griffiths, A. D. & Winter, G. Making antibody fragments using phage display libraries. Nature 352, 624-628 (1991).

### Methods

### Plasmid Construction

The backbone for all in vivo mRNA display plasmids and respective controls is pSH100 (URA3 selection marker; Addgene #45930)1. MS2 capsid protein (MCP) was PCR amplified from pSH1001, while stem-loop sequences from pDZ4151 were ordered as Blocks from IDT; defective MCP (MCP*) mutations were introduced via overlap PCR. For cloning, PCR insert fragments for MCP variants and a Gateway cloning ccdB cassette were amplified using Q5 polymerase (NEB M0491). The backbone was digested using restriction enzymes, combined with PCR inserts for Gibson Assembly according to the manufacturer (NEB E2611), and transformed into One Shot ccdB Survival Cells (Invitrogen A10460). The resulting Destination vectors (plPOIVMD156, 160, 155) allow for Gateway cloning of ORFs flanked by Gateway attL sites in to the display constructs. The in vivo mRNA display constructs in this study are under the control of a MET25 promoter (induced in methionine dropout media). Super folder GFP2 and mCherry1 were amplified with flanking attB sites and Entry Vectors were generated via a BP reaction. Individual yeast ORFs were amplified from the ORFeome collection purchased from Dharmacon3 using the available flanking attB sites. Plasmids expressing SAM2- and ARC40-GFP fusion baits were constructed using Gibson assembly based on the pSH624 backbone (HIS3 selection marker; plIVMD495, 496). DH5α competent cell were used for all bacterial transformations (NEB C2989K).

### Yeast Strains

The BY4742 S288c MATα laboratory deletion strain was used as the starting strain for all strains harboring in vivo mRNA display constructs. All plasmids were transformed using the LiAc-PEG-ssDNA method5, and selected in 2% glucose-HIS dropout media. EY0986 MATa deletion strains expressing genomically integrated SAM2- and ARC40-GFP fusions6 were purchased from Thermo Fischer (Catalog no. 95701; HIS3 selection marker). Plasmids expressing SAM2- and ARC40-GFP fusion baits were transformed into BY4741 S288c MATa laboratory deletion strain and selected on 2% glucose-HIS dropout media. For diploid strains, MATa and MATα haploids were mated in YPD at 30° C. with vigorous shaking for 1-2 hours and selected on 2% glucose-URA, -HIS dropout media. Dropout media supplement powders from For Medium and US Biological were used interchangeably. Single strain selections were plated on appropriate 2% hard agar SC dropout plates.

In Vivo mRNA Display Library Generation

E. coli strains from the yeast ORFeome plasmid collection were outgrown, pooled and pelleted. Pooled plasmid was extracted from the pellets using a Qiagen Maxi-prep kit (#12963). Yeast ORFs were PCR amplified using the attB1 and attB2 flanking sequences. A two-step recombination reaction (first a BP recombination into pDONR221, followed by an LR reaction) was used to transfer the sequence into the Gateway cloning site of the in vivo mRNA display Destination Vector (plIVMD156). BP and LR reactions were transformed in DH5α cells (NEB C2989K) and colonies were selected in semi-liquid soft agarose gel7 (0.3% Lonza Seaprep #50302) LB media with the appropriate antibiotics (kanamycin and ampicillin for BP and LR reactions respectively). More than 1 million colonies were collected for the BP reaction (˜200× coverage) and over 125,000 colonies (˜25× coverage) for LR reactions. The final in vivo mRNA display library was transformed into BY4742 using the LiAc-PEG-ssDNA method5, and selected in 2% glucose SC-URA semi-liquid soft agarose gel7 (0.3% Lonza Seaprep #50302). Over 500,000 colonies were collected, outgrown for 6 hours in SC-URA and stored at −80° C. in 15% glycerol media. Mated diploid libraries were selected in 2% glucose SC-URA-HIS semi soft agar7 at similar coverage. For both bacterial and yeast libraries, colony counts were assessed by plating a dilution on hard agar SC dropout media.

### Yeast Cell Culture

S. cerevisiae strains were cultured in the appropriate SC dropout media (-HIS, -URA or -HIS and -URA) supplemented with 2% glucose at 30° C. and shaken at 220 rpm. Overnight cultures were induced by seeding 0.1 OD600/ml into a new liquid culture with a similar SC dropout media additionally lacking methionine (-MET). Strains were outgrown for 6-8 hours to 0.6-0.8 OD600/ml and collected by centrifugation, washed twice with ultrapure water, and split in aliquots equivalent to 10 to 40 OD600 units of cultured cells. Pelleted cells were flash frozen in a dry ice ethanol bath and stored at −80° C. until further processing.

For in vivo mRNA display yeast libraries, biological replicates were independently revived from frozen stock and outgrown in semi-liquid soft agarose gel as colonies to avoid any growth biases. All resulting colonies were pooled and outgrown in liquid SC dropout media as described above.

### Excess Capsid Protein

Unless otherwise noted, an excess of capsid protein was provided for all high throughput experiments in order to titrate any non-specific interactions. To this end, into each sample an excess culture of yeast cells was missed that express an MCP fusion that does not display its own mRNA. Additionally, the MCP is not isolated specifically in any given protein assay and its mRNA is not processed during first strand and second strand synthesis.

For excess capsid expression, three strains (scIVMD115, 118, 217: MCP-mCherry-FLAG, MCP-mCherry-MYC, and MCP-BFP-HA) were constructed. Upon induction, the equivalent of 30 OD600 units of cells were mixed with 10 OD600 of in vivo mRNA display library cells immediately prior to or immediately after freezing. If purifying a FLAG- or MYC-tagged in vivo mRNA display library, the anti-FLAG or anti-MYC tagged excess capsids were excluded, respectively. For 6×HIS and GFP tag purifications, all three strains were mixed in equal proportions.

Moreover, lysis and all purification steps were performed at 4° C. since increased temperatures also compromise precision, possibly due to partner exchange (FIG. 13-14).

Non-Specific Functional Controls for In Vivo mRNA Display

A set of in vivo mRNA display constructs were included in every library that function as internal negative and positive controls for a given protein purification assay. Their mRNA frequencies provide a background with respect to which the frequencies of each ORF were normalized (FIGS. 11-12). For that, a small set of reporter genes and peptides was chosen that should not participate in any biological interactions inside the cell. These control ORFs included GFP2, mCherry1, BFP (Addgene #44839), acGFP (pBI-CMV2; Clontech), Firefly Luciferase, Renilla Luciferase (psiCHECK-2; Promega) as well as short peptides derived from these reporter genes. All ORFs were cloned in vectors with an N-terminal MCP, a downstream SL and various purification tags (MYC, FLAG, 6×HIS, HA-tag) such that a subset of them works as a non-specific control set for every protein purification. Moreover, during anti-GFP magnetic bead purifications, the GFP construct works as a positive control for the assay. Additionally, a smaller set of 7 control proteins was designed that harbored an MCP but no SL such that their mRNA progenitors function as non-displaying controls. These proteins are variants of mCherry with 6 additional bases on the 5′ and 3′ ends of the ORF, so that they can be identified during sequencing. Control strains were added at various concentrations to represent a wide range of construct frequencies and allow us to assess any representation biases and determine read depth cut offs. Overall, the controls represented 2-5% of the total processed cell culture.

Additionally, these constructs allow us to assess RNA integrity and the efficiency of our assay post purification but prior to sample preparation for sequencing by means of Quantitative PCR (see relevant Quantitative PCR Method section).

### Whole Cell Lysate Preparation

Frozen cell pellets were re-suspended in 750 μl of ice cold Lysis Buffer8 (20 mM HEPES pH 7.5, 140 mM KCl, 1.5 mM MgCl2, 1% Triton X-100, 1× Complete Mini Protease Inhibitor EDTA-free, 0.2 U/μl SUPERase RNase Inhibitor) and added on top of 250 μl of pre-chilled acid washed glass beads (Sigma G8772). After this point, samples were kept at 4° C. throughout all purification steps. The samples were homogenized9 in a Fast-Prep24 5G instrument (10 rounds of a 30 sec disruption pulse at 6 m/s followed by 5 minutes of rest in contact with ice cold ethanol packs in between disruptions). Glass beads were removed by a 1 min centrifugation at 7,000×g, and lysate was transferred to a new tube and further cleared by a 30 second spin at 11,000×g. Roughly 1001 of the resulting sample was set aside, which is referred to as the lysate.

In Vivo mRNA Display Library Purification & Protein Bait Purification

We used magnetic beads for all tagged protein purifications. Purifications were performed at 4° C. For 6×HIS tagged proteins, we used His Isolation Dynabeads (Invitrogen 10103D). For MYC tagged proteins, we used Anti-c-Myc Beads (Pierce #88842). For FLAG tagged proteins, we used Anti-FLAG M2 beads (Sigma M8823). For GFP and mCherry tagged proteins, we used GFP- and RFP-Trap beads respectively (ChromoTek gtma and rtma). All beads were washed 3 times before use with 200 μl of Wash Buffer (50 mM Sodium Phosphate pH8, 300 mM NaCl, 0.01% Tween-20, 0.02 U/μl SUPERase RNase Inhibitor). Beads in 200 μl of Wash Buffer were added to 400 μl of whole cell lysate and incubated on a roller (8 min for 6×HIS, 30 min for MYC, 2 hours for FLAG, 1 hour for GFP and RFP tagged proteins). Then beads were washed 4 times with 300 μl Wash Buffer and resuspended in 100 μl of Storage Buffer (same as Wash Buffer with 0.2 U/μl SUPERase RNase Inhibitor).

For 6×HIS purifications, 10 mM of Imidazole was added in both Lysis and Wash Buffers. Additionally, proteins were eluted in 300 mM of Imidazole and re-purified using a fresh aliquot of His Isolation Dynabeads (Invitrogen 10103D). To that end, 100 μl of eluted sample was mixed with 1000 μl of Wash Buffer and incubated on a roller, and washed 4 times.

### Crude Mitochondrial Isolation

We processed frozen library pellets (scIVMD580) equivalent to 20 OD600 units of cells per replicate. There was no excess capsid culture added to the yeast libraries for these purifications. We performed a crude mitochondrial isolation using a commercially available kit from Sigma (MITOISO3). The frozen library cell pellets were resuspended in 2 ml of ice cold Buffer A (Sigma B3311 with 0.02 U/μl SUPERase RNase Inhibitor) and incubated for 15 min at 30° C. with gentle shaking. Next, they were centrifuged at 1,500×g for 5 min, resuspended in 1 ml of Buffer B (Sigma B3186 with 0.02 U/μl SUPERase RNase Inhibitor), and supplemented with 40 units of Lyticase Solution (Sigma L2524). Spheroplasts were formed by incubating at 30° C. with gentle shaking for roughly 10 min (until OD600 decreases to 30% of the initial value). The reaction was stopped by centrifuging at 1,200×g for 5 min at 4° C. Spheroplasts were homogenized in 1 ml of Storage Buffer (Sigma S9689 with 0.2 U/μl SUPERase RNase Inhibitor) with 10 strokes using a pre-chilled sterile Dounce homogenizer at 4° C. (Sigma T2690; P1110). To remove nuclei, samples were centrifuged at 600×g for 10 min at 4° C. The supernatant was transferred to a new Eppendorf tube and centrifuged at 6,500×g for 10 min at 4° C. The supernatant was saved for further processing (75 μl for RNA extraction). Storage buffer was added to the pellet and the sample was centrifuged at 6,500×g for an additional 10 min at 4° C. Supernatant was discarded and the final pellet was saved for further processing.

### RNA Extraction

We extracted RNA from all protein samples (50 μl of whole cell extract; up to 100 μl of purified protein bound on beads; 75 μl of 6,500×g supernatant from crude mitochondrial isolation; or the complete 6,500×g pellet) using Trizol (Invitrogen 15596026). We added 750 μl of Trizol reagent to each sample, vortexed and incubated at RT for 5 min. We added 150 μl of chloroform (Sigma C2432), vortexed and incubated for 2 min at RT. Samples were centrifuged at 12,000×g for 15 min at 4° C. The aqueous phase was transferred to a new tube and mixed with an equal volume of 100% ethanol. RNA was washed and concentrated using spin columns (Zymo Research, RNA Clean & Concentrator R1015). RNA from lysates was eluted in 50 μl, while RNA from purified protein samples was eluted in 15 μl of RNase free water with 0.2 U/μl SUPERase RNase Inhibitor.

cDNA Synthesis

For whole cell extract samples, we used 8 μg of purified total RNA as input. For purified protein samples, we used the whole sample. We treated extracted RNA with dsDNase (Thermo Scientific EP0771) at 37° C. for 5 min in a 20 μL reaction (volumes doubled from manufacturer's recommendation). DNase treated RNA was reverse transcribed using Maxima H Minus RT (Thermo Scientific EP0752) and a construct specific primer (prIVMD212) binding downstream of the in vivo mRNA display construct ORF (FIG. 6). The samples were incubated at 65° C. for 5 min with RT primer (prIVMD212) and dNTP mix per the manufacturer's recommendations. RT Buffer and RT Enzyme were added and samples were incubated for 30 min at 50° C. The reaction was terminated at 85° C. for 5 min. When random hexamers were used (FIGS. 1D-E) the 50° C. incubation was preceded by a 10 min incubation at 25° C. Next, we hydrolysed remaining RNA by adding 8 μl of 500 mM EDTA and 8 μl of 1N NaOH per 40 μl of 1st Strand Synthesis samples and incubating at 65° C. for 15 min. cDNA was cleaned and concentrated using a Zymo Research spin column kit (D4013) by adding 7 volumes of binding buffer and washing twice. Samples were eluted in 20 μl of DNase free water.

For second strand synthesis, we performed a PCR amplification using construct specific primers upstream and downstream of the in vivo mRNA display ORF (prIVMD113 & prIVMD212, FIG. 6) and PrimeSTAR GXL DNA polymerase (Clontech R050B). We set up 50 μl reactions according to the manufacturer's recommendations for the Rapid PCR protocol (2× enzyme) with annealing at 58° C. and 90 second extension for 8 cycles. Second strand synthesis samples were purified using a Zymo Research spin column kit (D4013) by adding 5 volumes of binding buffer and washing twice. Samples were eluted in 20 μl of RNase free water.

### Quantitative PCR

We assessed extracted RNA for quality and in vivo mRNA display efficiency using qPCR. Quantitative PCR (PerfeCTa SYBR Green FastMix, QuantaBio 95073-012; on an Applied Biosystems QuantStudio5 384-well instrument) was used to determine the relative abundance of mCherry and GFP transcript in each sample. Protein purification experiments were designed such that either GFP or mCherry is co-purified in the experiment (specific positive control) and the other is washed away (non-specific reference). We calculated a ΔCt value for each sample and a −ΔΔCt between purified sample and input lysate. Therefore the Log 2 Fold Enrichment is:

−ΔΔCₜ=[Cₜ^{Specific}−Cₜ^{Non-Specific}]^{IP}−[Cₜ^{Specific}−Cₜ^{Non-Specific}]^{LYS}.

For random hexamer RT (FIG. 1D), the relative abundance to ACT1 was quantified as a reference. For each sample, technical duplicate replicate measurements were made, and if they were inconsistent, they were repeated for quadruplicates resulting in the reporting of average values and standard deviations as error bars (FIGS. 4, 12, 13, 14). For FIGS. 1D-E, purification experiments were conducted in biological replicates, and averages of biological replicates are reported as bars and replicate values as grey dots.

In Vivo mRNA Display Library Sequencing Preparation

Restriction Enzyme Digestion: To prepare samples for sequencing we used 20 μl of double stranded cDNA as input. Each sample was split in half for two 20 μl restriction enzyme digestion reactions (FIGS. 6-8). One half was treated with HinP1I (NEB R0124) and AciI (NEB R0551), while the other half was treated with MspI (NEB R0106) and HpyCH4IV (NEB R0619). Each 20 μl digestion contained 1 μl of each restriction enzyme and 2 μl of CutSmart Buffer (NEB B7204) and was incubated at 37° C. for 3-6 hours and heat inactivated at 65° C. for 20 min. Reactions were combined and purified using a Zymo Research spin column kit (D4013) by adding 7 volumes of binding buffer and washing twice. Each sample was eluted in 9 μl of DNase free water. All restriction enzymes generate a CG overhang used for linker ligation.

Y-Linker Annealing: Per 8 samples, we used 8 μL of HPLC purified 100 μM YCG5 and 8 μL of 100 μM YCG3 primer, combined with 2 μL of DNase free water and 2 μL of 10× Annealing Buffer (1M NaCl, 100 mM Tris-HCl ph8, 10 mM EDTA pH8)10. Samples were placed in a thermocycler and with a starting temperature of 94° C. and slowly cooled to 25° C. (reduced by 2° C. every 30 seconds).

Y-Linker Ligation: For each sample, 9 μl of cleaned up digestion was mixed with 2.5 μl of annealed Y-Linker, 1 μl of Quick Ligase (NEB M2200) and 12.5 μl of 2× Quick Ligase Buffer. The reaction was incubated at room temperature for 10 min. We added 1 μl of 500 mM EDTA to stop the reaction and purified using a Zymo Research spin column kit (D4013).

Multiplexing and NGS adapter addition: We set out to amplify the ligated 5′ and 3′ ends on each ORF. For 5′ fragments, one primer lands on the universal sequence of in vivo mRNA display constructs upstream of the ORF (FIG. 6) and the other lands on the ligated Y-Linker. For 3′ fragments, one primer lands on the universal sequence of in vivo mRNA display constructs downstream of the ORF and the other lands on the ligated Y-Linker. In the process of amplification, Illumina adapters are added for Next Generation Sequencing and samples are multiplexed. We perform this amplification in two rounds of PCR amplification.

PCR amplification Round 1: During the first round, custom-designed identifying index sequences of varying length were included on the end of the PCR that would be sequenced, as well as partial Illumina adapter sequences on both ends. The custom-designed indexes are used to multiplex samples but also to stagger the library sequences to achieve the necessary variability in the initial bases (because all our library sequences included an identical universal adaptor at each end of the ORF). Two PCRs are set up for every sample: one amplifying the 5′ end of every ORF and one amplifying the 3′ end of every ORF in the library. For each 5′ or 3′ ORF PCR, one primer lands on the universal construct sequence that is upstream or downstream of the 5′ or 3′end of each ORF, respectively, while the other PCR primer lands on the Y-Linker (FIG. 6). PCR amplification was performed for 7 cycles using the Q5 High Fidelity Polymerase (NEB M049; a two PCR program with annealing at 62° C. for the first 3 cycles and 67° C. for the remaining 4 cycles and 2 minute extension throughout). Reactions were set up as per the manufacturer's recommendations. Upon completion of thermocycling reaction, we combined 5′ and 3′ PCRs and used Ampure XP beads for DNA cleanup (A63881, Beckman Coulter, Brea, Calif.) at a 1.7× ratio. We eluted fragments in 25 μl of water.

PCR amplification Round 2: During the second round, Illumina Adapter sequences were extended while Illumina indexes were added to each sample for further multiplexing. Reactions were set up using the Q5 High Fidelity Polymerase (NEB M04) as per the manufacturer's recommendations. A 40 μl reaction was set up side by side with a smaller 10 μl reaction additionally including ROX Low Reference Dye (KK4602, Kapa Biosystems, Wilmington, Mass.) and SYBR dye (EvaGreen; 31000, Biotium, Fremont, Calif.) in 1× concentrations. The smaller reaction was split in two technical replicates and cycled on a qPCR machine. Amplification was observed to determine the number of cycles needed or the amplification to reach the exponential phase (or roughly 30% of the maximum signal) and the number of cycles were noted11. The remaining 40 μl PCR reaction was thermocycled for the same number of cycles as noted from the qPCR. A two-step PCR program was employed for both qPCR and regular PCR with annealing at 65° C. for the first 3 cycles and 68° C. for the remaining cycles and 90 second extension throughout. We used Ampure XP beads (A63881, Beckman Coulter, Brea, Calif.) at a 1.3× ratio. We eluted fragments in 25 μl of water.

The concentration of each sample was measured using the Qubit dsDNA HS Assay Kit (Q32854, Invitrogen) and/or the Agilent Bioanalyzer High Sensitivity DNA kit (5067-4626, Agilent, Santa Clara, Calif.). Libraries were sequenced for 75 cycles with the NextSeq 500/500 High Output Kit v2.5 (20024906, Illumina) either single-end or pair-end depending on the needs of other libraries on the lane. For pair-end sequenced samples, cycles were allocated as follows: 58 cycles read 1, 17 cycles read 2. Only read 1 was utilized for data analysis (read 2 contains a universal Y-Linker sequence).

In Vivo mRNA Display Sequencing Data Analysis

After de-multiplexing Illumina indexes, we used Cutadapt12 to trim low-quality sequences and trim universal 3′ adapter sequences corresponding to the Y-Linker primers. Cutadapt was also used to de-multiplex internal custom indexes, and remove universal 5′ adapter sequences. Surviving reads of sufficient length (>20 nt) were mapped to the 5′ and 3′ ends of all yeast ORFs using Bowtie13. If the sum of all reads mapping to the 5′ end (or the 3′ end) of each ORF gᵢ is Rᵢ⁵′ (or Rᵢ³′), we can calculate an average log frequency, for the 5′ fragments, the 3′ fragments and the average:

$${f}_{i}^{5 ′} = {{log}_{2} [ \frac{{R}_{i} + 1}{{\sum }_{j} {R}_{j}} ]}^{5 ′} ,$$

$${f}_{i}^{3 ′} = {{log}_{2} [ \frac{{R}_{i} + 1}{{\sum }_{j} {R}_{j}} ]}^{3 ′} ,$$

$${f}_{i} = 0.5 ( {f}_{i}^{5 ′} + {f}_{i}^{3 ′} ) .$$

for ORF gᵢ, where ΣⱼRⱼ represents the total sum of reads for all the 5′ (or 3′) end reads. If C is the set of constructs in the non-specific functional control library, we calculate a log normalized frequency, nⁱ, for each ORF gⁱ with respect to the control set:

$${n}_{i}^{5 ′} = {f}_{i}^{5 ′} - \frac{{\sum }_{k ∈ C} {f}_{k}^{{5}^{′}}}{\vert C \vert} , {n}_{i}^{{3}^{′}} = {f}_{i}^{{3}^{′}} - \frac{{\sum }_{k ∈ C} {f}_{k}^{{3}^{′}}}{\vert C \vert} , {n}_{i} = {f}_{i} - \frac{{\sum }_{k ∈ C} {f}_{k}}{\vert C \vert} .$$

The Display Score for each ORF between two matched samples is calculated as the difference of the log normalized frequencies between the samples. For example, the Display Scores for ORF g\hat{ }i for a protein purification experiment are:

DSᵢ⁵′=nᵢ^{5′,Pur}−nᵢ^{5′,Lys},

DSᵢ³′=nᵢ^{3′,Pur}−nᵢ^{3′,Lys},

DSᵢ=nᵢ^{Pur}−nᵢ^{Lys},

where nᵢ^{Pur} is the log normalized frequency in the purified protein sample and nᵢ^{Lys} is the log normalized frequency in the input whole cell extract. An ORF is considered to be present in an experiment only if it had more than 8 reads in either the input or the purified sample (for FIGS. 2D-G a threshold of 4 reads was set based on the distribution of reads). An ORF is considered present in an assay with replicates, if it is present in half or more of the 3′ and 5′ samples of all the replicates. The Display score represents an enrichment (DSᵢ>0) or depletion (DSᵢ<0) of the reads of g\hat{ }i in the purified sample compared to the lysate with respect to the non-specific functional controls. The distribution of the non-specific functional controls can be used to calculate a z Score for the Display Score:

$${Z}_{i} = \frac{{DS}_{i}}{{σ}_{DS}^{controls}} ,$$

where σ_{DS}^{controls} is the standard deviation of the Display Scores of the non-specific functional controls, and μ_{DS}^{controls}=0 by definition. Z Scores for biological replicate experiments were averaged using the Stouffer rule. Display Score p-values for biological replicates (FIGS. 2D-G and FIG. 3) were calculated by comparing the distribution of DSᵢ⁵′ and DSᵢ³′ measurements for every ORF to the distribution of Display Scores for all the non-specific functional controls using a Mann-Whitney U test.

### Western Blots

We treated 10 μl of Lysate and 40% of the purified protein bound beads in 2×SDS Sample Buffer (Invitrogen LC2676) buffer and 10% β-mercaptoethanol at 95° C. for 10 min. The beads were separated on a magnetic stand. Each sample was split in half and each half was loaded on an Invitrogen WedgeWell 8 to 16% Tris-Glycine Mini Gel (Invitrogen XP08162BOX) for electrophoresis. Resolved proteins were transferred onto a nitrocellulose membrane, followed by 1 hr blocking with 1% milk TBS-T. One membrane was incubated overnight at 4° C. with one of the primary antibodies against GFP (Rat monoclonal ChromoTek 3H9; 1:1000), and the other against RFP (Mouse monoclonal ChromoTek 6G6; 1:2000). The membranes were washed and incubated at room temperature for 1 hour with HRP-conjugated secondary anti-Mouse and anti-Rat (Jackson Immuno Research 115035072 and 112035072). Proteins in blots were detected using the KwikQuant Detection Kit (Kindle Biosciences, R1004). RFP gels were stripped and re-probed with an α-Tubulin antibody conjugated to HRP (Rat monoclonal YOL1/34; Santa Cruz Biotechnology 53030; 1:500) and visualized again. Western Blot images were converted to greyscale and image colors were inverted.

### Mass Spectrometry

Immunoprecipitation and in-gel digestion: SAM2- and ARC40-GFP were purified as described above from in vivo display libraries using anti-GFP magnetic beads. Samples were processed in biological duplicate. Protein-bound beads were washed three times with ultrapure water. Samples were processed at the Proteomics and Macromolecular Crystallography Core Facility at Columbia University Medical Campus. Immunoprecipitated samples were separated on 4-12% gradient SDS-PAGE, and stained with SimplyBlue (Thermo fisher Scientific). Protein gel slices were excised and in-gel digestion was performed. Gel slices were washed with 1:1 (Acetonitrile: 100 mM ammonium bicarbonate) for 30 min, Gel slices were then dehydrated with 100% acetonitrile for 10 min until gel slices were shrink and excess acetonitrile was removed and slices were dried in speed-vac for 10 min at no heat. Gel slices were reduced with 5 mM DTT for 30 min at 56° C. in an air thermostat and chilled to room temperature, then alkylated with 11 mM IAA for 30 min in the dark. Gel slices were washed with 100 mM ammonium bicarbonate and 100% acetonitrile for 10 min each. Excess acetonitrile was removed and dried in speed-vac for 10 min at no heat and gel slices were rehydrated in a solution of 25 ng/μl trypsin in 50 mM ammonium bicarbonate on ice for 30 min on ice. Digestions were performed overnight at 37° C. in an air thermostat. Digested peptides were collected and further extracted from gel slices in extraction buffer (1:2 vol/vol) 5% formic acid/acetonitrile) at high speed shaking in an air thermostat. Supernatant from both extractions were combined and dried down in a speed-vac. Peptides were dissolved in 3% acetonitrile/0.1% formic acid.

LC-MS/MS analysis: Thermo Scientific™ UltiMate™ 3000 RSLCnano system and Thermo Scientific EASY Spray™ source with Thermo Scientific™ Acclaim™ PepMap™ 100 2 cm×75 μm trap column and Thermo Scientific™ EASY-Spray™ PepMap™ RSLC C18 50 cm×75 μm ID column were used to separate desalted peptides with a 5-30% acetonitrile gradient in 0.1% formic acid over 100 min at a flow rate of 250 nL/min. The column temperature was maintained at a constant 50° C. during all experiments. Thermo Scientific™ Orbitrap Fusion™ Tribrid™ mass spectrometer was used for peptide MS/MS analysis. Survey scans of peptide precursors were performed from 400 to 1500 m/z at 120K FWHM resolution (at 200 m/z) with a 2×105 ion count target and a maximum injection time of 50 ms. The instrument was set to run in top speed mode with 3 s cycles for the survey and the MS/MS scans. After a survey scan, tandem MS was performed on the most abundant precursors exhibiting a charge state from 2 to 6 of greater than 5×103 intensity by isolating them in the quadrupole at 1.6 Th. CID fragmentation was applied with 35% collision energy and resulting fragments were detected using the rapid scan rate in the ion trap. The AGC target for MS/MS was set to 1×104 and the maximum injection time limited to 35 ms. The dynamic exclusion was set to 45 s with a 10 ppm mass tolerance around the precursor and its isotopes. Monoisotopic precursor selection was enabled.

Data Analysis: Raw mass spectrometric data were processed and searched using the Sequest HT search engine within the Proteome Discoverer 2.2 (PD2.2, Thermo Fisher) with a reference Saccharomyces cerevisiae proteome database downloaded from SGD. The default search settings used for protein identification in PD2.2 searching software were as follows: two mis-cleavages for full trypsin with fixed carbamidomethyl modification of cysteine and oxidation of methionine and deamidation of asparagine and glutamine and acetylation on N-terminal of protein were used as variable modifications. Identified peptides were filtered for maximum 1% false discovery rate using the Percolator algorithm in PD 2.2. PD2.2 output combined folder uploaded in Scaffold (Proteome Software) for data visualization. Spectral counting was used for analysis to compare samples and p-values for the enrichment of every protein were calculated using Fisher's exact test for the combined counts of every protein in the SAM2 and the ARC40 samples.

### GO Term Analysis for Crude Mitochondrial Isolation

We considered mutually exclusive GO Term categories. For cytosolic, cytoplasmic and nuclear proteins, we filtered out the genes common to other organelle and membrane fractions (GO:0016020, ‘GO:0005739’, ‘GO:0005740’, ‘GO:0007005’, ‘GO:0005773’, ‘GO:0005777’, ‘GO:0016020’, ‘GO:0005783’, ‘GO:0005794’, ‘GO:0005635’, ‘GO:0005618’, ‘GO:0009277’, ‘GO:0005811’, ‘GO:0005768’, ‘GO:0005886’, ‘GO:0005743’, ‘GO:0005741’, ‘GO:0005759’, ‘GO:0005758’) and vice versa. P-values for enrichments and depletions were calculated using the hypergeometric test between the number of ORFs with significant Display Scores in each category compared to the significant Display Scores present in the assay (FIG. 3B, FIG. 24). We calculated calculate enrichment of genes in organelle and membrane categories with respect to cytosolic (GO:0005829) proteins.

### References for Methods

1. Hocine, S., Raymond, P., Zenklusen, D., Chao, J. A. & Singer, R. H. Single-molecule analysis of gene expression using two-color RNA labeling in live yeast. Nat. Methods 10, 119-121 (2013).
2. Pédelacq, J.-D., Cabantous, S., Tran, T., Terwilliger, T. C. & Waldo, G. S. Engineering and characterization of a superfolder green fluorescent protein. Nat. Biotechnol. 24, 79-88 (2006).
3. Gelperin, D. M. et al. Biochemical and genetic analysis of the yeast proteome with a movable ORF collection. Genes Dev. 19, 2816-2826 (2005).
4. Gueldener, U., Heinisch, J., Koehler, G. J., Voss, D. & Hegemann, J. H. A second set of loxP marker cassettes for Cre-mediated multiple gene knockouts in budding yeast. Nucleic Acids Res. 30, e23 (2002).
5. Daniel Gietz, R. & Woods, R. A. Transformation of yeast by lithium acetate/single-stranded carrier DNA/polyethylene glycol method. in Methods in Enzymology vol. 350 87-96 (Elsevier, 2002).
6. Huh, W.-K. et al. Global analysis of protein localization in budding yeast. Nature 425, 686-691 (2003).
7. Elsaesser, R. & Paysan, J. Liquid gel amplification of complex plasmid libraries. BioTechniques 37, 200-202 (2004).
8. Freeberg, M. A. et al. Pervasive and dynamic protein binding sites of the mRNA transcriptome in Saccharomyces cerevisiae. Genome Biol. 14, R13 (2013).
9. Szymanski, E. P. & Kerscher, O. Budding yeast protein extraction and purification for the study of function, interactions, and post-translational modifications. J. Vis. Exp. JoVE e50921 (2013) doi:10.3791/50921.
10. Hottes, A. K. & Tavazoie, S. Microarray-Based Genetic Footprinting Strategy to Identify Strain Improvement Genes after Competitive Selection of Transposon Libraries. in Strain Engineering (ed. Williams, J. A.) vol. 765 83-97 (Humana Press, 2011).
11. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
12. Martin, M. Cutadapt removes adapter sequences from high-throughput sequencing reads. EMBnet.journal 17, 10 (2011).
13. Langmead, B. & Salzberg, S. L. Fast gapped-read alignment with Bowtie 2. Nat. Methods 9, 357-359 (2012).
14. Ashburner, M. et al. Gene ontology: tool for the unification of biology. The Gene Ontology Consortium. Nat. Genet. 25, 25-29 (2000).
15. The Gene Ontology Consortium. The Gene Ontology Resource: 20 years and still GOing strong. Nucleic Acids Res. 47, D330-D338 (2019).
16. Morgenstern, M. et al. Definition of a High-Confidence Mitochondrial Proteome at Quantitative Scale. Cell Rep. 19, 2836-2852 (2017).

### Example 2—Demonstration of In Vivo mRNA Display in Mammalian Cells

To demonstrate in vivo mRNA display functionality in mammalian cells, a vector for expression of in vivo mRNA displayed proteins in human cells was generated. This construct allows for both transient expression and genomic integration, expressing an MCP-open reading frame (ORF) fusion that includes a short polypeptide purification tag, and is followed by a single copy of the 19-nt stem loop such that, upon translation, the fusion product binds to its encoding mRNA. The MS2 coat protein (MCP) was codon optimized for expression in human cell lines. The in vivo mRNA display construct is constitutively expressed under a hybrid human cytomegalovirus (CMV)/TetO2 promoter for high-level expression in a wide range of mammalian cells. As a backbone for the mammalian in vivo mRNA display system, we used the pcDNA™ FRT/TO vector. The vector allows for stable expression using Flp recombinase-mediated integration of the vector into Flp-In™ T-REx™ host cell lines. Following transfection and hygromycin selection, each cell contains a single species of the in vivo mRNA displayed construct corresponding to a single displayed protein, which interacts with its cellular context independently from all of the other species in the library. For transient expression, a single construct can be transfected in a cell line of interest. Once the in vivo mRNA display protein has been expressed, stably expressed or transiently transfected cells can be assayed according to the desired biochemical assay, which is expected to preserve the RNA-protein linkage. The enrichment/depletion of each ORF sequence can be quantified by comparing their abundance in isolated RNA before and after the assay.

For mammalian in vivo mRNA display, we transiently transfected adherent human embryonic kidney cells with a set of constructs expressing MCP fluorescent protein fusions. Each construct was transfected to the cell line independently. The expressed fusion protein was immunoprecipitated using magnetic beads that specifically recognize each construct. Immunoprecipitation (IP) of the target protein copurifies its self-identifying mRNA with an enrichment of hundred-fold relative to the input lysate as measured by RT-PCR over a construct missing the downstream stem loop (FIGS. 31A-C). In contrast, a defective coat protein construct, MCP* (N55D, K57E), shows no enrichment of its respective mRNA upon purification (FIG. 31A).

### Methods

For transfections, Flp-In™ 293 T-REx cells were grown on 10 cm plates in D10F media up to 70% confluency. Transfections were performed using Lipofectamine 2000 (Invitrogen 11668-019) according to the manufacturer's recommendations. 48 hours post transfection, cells were washed once in PBS and harvested on ice by scrapping and pelleted at 4° C. Pelleted cells were flash frozen in a dry ice ethanol bath and stored at −80° C. until further processing. Human whole cell lysate was prepared and proteins were purified identical to the S. cerevisiae protocol.

### Example 3—Functionally Characterizing Protein Domains with Single Amino Acid Resolution

In vivo mRNA display technology can be used for the in vivo characterization of protein domains, in vivo screening for protein engineering, and selection of peptides that bind to a given target (biopanning). In vivo mRNA display can be used to perform high-throughput functional assays for peptide libraries and designed or mutagenized ORF collections.

For example, using a collection of constructs encoding in vivo mRNA displayed combinatorial or rationally synthesized peptide libraries one can perform in vivo selections for a given function, for example binding to a given target or localization to a given compartment. In vivo mRNA display can also be used to characterize protein function at the single amino acid resolution. Using a collection of randomly or systematically modified ORFs encoding in vivo mRNA displayed variants of a given protein, one can perform systematic characterization of protein domains that are required for diverse protein functions including, but not limited to expression, folding, stability, enzymatic activity, signaling, regulatory functions, sub-cellular localization, and interactions with other proteins.

To demonstrate the ability of in vivo mRNA display technology to screen for protein function with single amino acid resolution, we generated mutagenized libraries for a given ORF encoding a specific protein. Using error-prone PCR we generated two independent mutagenized libraries for S. cerevisiae protein ARC35. This protein is known to participate in the ARP2/3 complex which is required for the motility and integrity of cortical actin patches. ARC35 is evolutionarily conserved and contains a protein family domain that spans almost the entirety of the protein (PF04045). For the mutants included in our library, each protein variant contains on average one randomly generated single point mutation. Inevitably some mutations are synonymous, some are nonsense and some are non-synonymous mutations. How these mutations affect the participation of ARC35 in the ARP2/3 complex was investigated by purifying the complex using magnetic beads against ARC40, another member of the complex. After induction and homogenization of this in vivo mRNA display library of ARC35 variants, RNA reads throughout the entire mutagenized ORF from the lysate were compared with the purified sample. The frequency of each mutation in both samples was determined and a corresponding depletion score was calculated.

For a given single nucleotide mutation, it was determined that the variant affects the functional participation of the protein in the complex if it is significantly depleted in the purified sample compare to the lysate (Log 2 Fold Depletion of less than −2). Hundreds of mutants per mutagenized library generated were characterized. Synonymous mutations are not expected to affect protein function. Non-synonymous mutations were found to be significantly more likely to be depleted compared to synonymous mutations, and thus, more likely to disrupt the function of the protein (FIGS. 32A and D). As expected, all nonsense mutations within the functional domain of the protein had significant depletion scores (FIGS. 31B and E) as they introduce an early stop codon preventing the remaining of the domain from being translated. Overall, this experiment characterized hundreds of single amino acid substitutions. While the vast majority of substitutions does not appear to have a functional effect, we unveiled tens of such substitutions across ARC35 that significantly affect the functional co-purification of ARC35 with ARC40 (FIGS. 32C and F).

In some embodiments, the nucleotide sequence encoding the protein of interest includes one or more deletions, insertions, or mutations as compared to its wild-type sequence. In some embodiments, the protein of interest encoded by the nucleotide sequence includes one or more deletions, insertions, or mutations as compared to its wild-type sequence. In some embodiments, the one or more one or more deletions, insertions, or mutations is generated using random mutagenesis techniques. In some embodiments, the one or more one or more deletions, insertions, or mutations is generated using rational synthesis techniques. In some embodiments, the variant library includes in silico designed ORFs. In some embodiments, the variant library includes in silico designed peptides. In some embodiments, the variant library includes rationally designed ORFs. In some embodiments, the variant library includes rationally designed peptides.

### Example 4—In Vivo mRNA Display Using Universal RNA Barcodes

In one embodiment, the subject matter described herein relates to in vivo mRNA display using UMI. In some embodiments of the technology described herein, a specific in vivo displayed protein is attached to an identifying sequence other than the ORF encoding the protein itself. In this embodiment of the technology, individual cells concurrently express: 1) a single protein (from the library) fused to the RNA-binding domain (e.g. stem-loop recognition domain) and 2) a hybrid mRNA molecule containing both a unique sequence (bar-code) and the RNA stem-loop that is recognized by the RNA-binding domain. This can enable the generation of in vivo mRNA displayed proteins that are attached to a unique (typically much shorter) RNA bar-code sequence (as opposed to the variable length ORF encoding the protein itself.) It is envisioned that this will enable generation of in vivo mRNA display libraries with much shorter RNA molecules of standard length.

### Example 5—Using In Vivo mRNA Display to Systematically Characterize or Engineer Protein Domains Required for a Variety of Protein Functions

In one embodiments, the subject matter described herein relates to using in vivo mRNA display to systematically characterize or engineer protein domains required for a variety of protein functions. An in vivo mRNA display library focused on a population of variants of a single protein or a peptide library can enable systematic discovery of protein domains that are required for diverse protein function including expression, folding, stability, enzymatic activity, signaling, regulatory functions, sub-cellular localization, and interactions with other proteins or targets. These protein variant libraries can be generated by random mutagenesis or rationally synthesized to explore specific regions of the protein and sequence-space within.

### Example 6—Detecting all-Against-all Protein-Protein Interactions in a Library of In Vivo mRNA Display Proteins

In one embodiments, the subject matter described herein relates detecting all-against-all protein-protein interactions in a library of in vivo mRNA display proteins. Protein-protein interactions among a population of in vivo mRNA display proteins can be detected by utilizing proximity-based methods (e.g. proximity ligation) to generate hybrid sequences between the encoding nucleic-acid tag sequences encoding the two ORFs (or alternatively, the bar-codes representing them) that have been brought into close physical proximity of each other due to the specific interaction of two proteins. These hybrid sequences can be used to identify and quantify protein-protein interactions among the library members.

These hybrid sequences, once identified through DNA sequencing, can be used to identify and quantify protein-protein interactions among the library members. This can be accomplished using standard molecular biology protocols. In some embodiments, molecular ligation of the two in vivo mRNA display species can be achieved using standard proximity-ligation protocols (for example: Ramani et al. Nature Biotechnology, 33, 980-984 (2015)). In such a scheme, the 7-methylguanosine cap (m⁷G) of mRNAs can be removed by using MDE (mRNA decapping enzyme, available from NEB) (Paquette, D R., et. al (2018). RNA. 24 (2), 251-257), exposing 5′ monophosphate ends. Then T4 RNA ligase I (available from NEB) can be used to catalyze inter-molecular ligation between the 5′ and 3′ ends of two distinct in vivo mRNA displayed proteins that have been brought into close proximity of each other through the specific interaction of the two proteins. This generates a hybrid RNA molecule containing the sequences from the two ORFs (or alternatively the UMIs representing them) which can be reverse transcribed to DNA and DNA sequenced in order to identify the interacting proteins within a diverse pool of potentially interacting in vivo mRNA displayed protein interactants in the library.

### Example 7—Detecting all-Against-all Protein-RNA Interactions Among the Members of an In Vivo mRNA Display Library and a Population of RNA Molecules

Proximity-ligation based methods can be used to generate hybrid sequences between the encoding in vivo mRNA display ORF sequence (or alternatively, the UMI representing the ORFs) and the sequence of any one of a population of diverse RNA molecules in a diverse pool of potential interactants. These hybrid sequences can be used to identify and quantify protein-DNA or protein-RNA interactions among the library members. In one instantiation, this can be achieved using standard proximity ligation protocols (for example: Ramani et al. Nature Biotechnology, 33, 980-984 (2015)). In such a scheme, T4 RNA ligase I (available from NEB) can be used to catalyze inter-molecular ligation between the 3′ end of the mRNA displaying a protein and the 5′ end of an RNA molecule specifically interacting with the displayed protein that have been brought into close proximity of each other through the specific protein-RNA interaction. This generates a hybrid RNA molecule containing the sequence from the ORF (or alternatively, the barcode representing the ORF) and the sequence of the RNA that is specifically interacting with the protein encoded by the ORF. This hybrid RNA molecule can then be reverse transcribed into DNA and sequenced in order to identify the specific RNA-protein interaction with the diverse pool of potentially interacting in vivo mRNA displayed proteins and RNA molecules.

### Example 8—Detecting all-Against-all Protein-DNA Interactions Among Members of an In Vivo mRNA Display Library and a Population of DNA Molecules

Proximity ligation based methods can be used to generate hybrid sequences between the ORF sequence encoding the in vivo mRNA displayed protein (or alternatively, the barcode representing the ORF) and the sequence of any one of a population of diverse DNA molecules in a diverse pool of potential interactants. These hybrid sequences can be used to identify and quantify specific protein-DNA interactions between the displayed proteins and any of the DNA molecules in a diverse pool. These hybrid ligation products can be generated using standard molecular biology protocols. In one instantiation, MMLV reverse transcription from the mRNA of the in vivo mRNA display species can generate complementary cDNA extending to the 5′ end of the mRNA and adding three protruding nucleotides (CCC) to the 3′ end of the nascent cDNA (Zhu Y Y et al. Biotechniques, 30(4):892-897 (2001)). The library of potentially interacting double-stranded DNA molecules can be first prepared by ligating a double-stranded linker to their ends, which contains a protruding 3′ end with three guanosine nucleotides (GGG-3′). Upon a specific interaction of a DNA molecule in the pool with a specific displayed protein, the end of the DNA can be efficiently ligated (e.g. T4 DNA ligase) to the 3′ end of the nascent cDNA via the complementarity between the protruding CCC on the cDNA and GGG on the DNA interactant, forming a hybrid DNA sequence which can be PCR amplified and sequenced in order to reveal the identity of the protein and the interacting DNA within the diverse pool of potential interactants.

### Example 9—Quantifying Global Phosphorylation Dynamics of the Proteome

By using phosphor-specific antibodies, one can capture the dynamic nature of proteome phosphorylation in response to environmental or genetic perturbations (e.g. by modulating the activity of a kinase or phosphatase).

What is claimed:

1. A nucleic acid comprising a mRNA display cassette, the mRNA display cassette comprising a cloning site for insertion of a nucleotide sequence encoding a protein of interest operably linked to (i) a nucleotide sequence encoding a MS2 bacteriophage coat protein (MCP) and (ii) to a nucleotide sequence encoding an RNA stem-loop, wherein the MCP binds to the RNA stem-loop with high-affinity.

2. A nucleic acid comprising (i) first cassette comprising a cloning site for insertion of a nucleotide sequence encoding a protein of interest operably linked to a nucleotide sequence encoding a MS2 bacteriophage coat protein (MCP) and (ii) a second cassette comprising a nucleotide sequence encoding a unique molecular identifier (UMI) sequence operably linked to a nucleotide sequence encoding an RNA stem-loop, wherein the MCP binds to the RNA stem-loop with high-affinity.

3. The nucleic acid of claim 1 or 2, wherein the nucleotide sequence encoding the MCP is located 5′ to the cloning site for insertion of the nucleotide sequence encoding the protein of interest.

4. The nucleic acid of claim 1 or 3, wherein the nucleotide sequence encoding the RNA stem-loop is located 3′ to the cloning site for insertion of the nucleotide sequence encoding the protein of interest.

5. The nucleic acid of claim 4, wherein the nucleotide sequence encoding the RNA stem-loop is located in a 3′ UTR.

6. The nucleic acid of claim 1, wherein the mRNA display cassette is configured so that upon insertion of a nucleotide sequence encoding a protein of interest, the nucleotide sequence encoding the protein of interest and the MCP are operably linked so that they encode a fusion protein of the protein of interest and the MCP.

7. The nucleic acid of claim 6, wherein the fusion protein comprises the MCP fused to the N-terminus of the protein of interest.

8. The nucleic acid of any one of claim 1-7, wherein the mRNA display cassette further comprises a nucleotide sequence encoding a purification tag operably linked to the cloning site for insertion of the nucleic acid sequence encoding the protein of interest.

9. The nucleic acid of claim 8, wherein the mRNA display cassette is configured so that upon insertion of a nucleotide sequence encoding a protein of interest, the nucleotide sequence encoding the protein of interest and the purification tag are operably linked so that they encode a fusion protein of the protein of interest and the purification tag.

10. The nucleic acid of claim 9, wherein the fusion protein comprises the purification tag fused to the C-terminus of the protein of interest.

11. The nucleic acid of any one of claims 1-10, further comprising a promoter operably linked to the mRNA display cassette.

12. The nucleic acid of claim 11, wherein the promoter is an inducible promoter.

13. The nucleic acid of any one of claims 1-12, wherein the mRNA display cassette further comprises a nucleotide sequence encoding a protein of interest.

14. The nucleic acid of claim 13, wherein the nucleotide sequence encoding the protein of interest comprises one or more deletions, insertions, or substitutions as compared to its wild-type sequence.

15. The nucleic acid of claim 13, wherein the protein of interest comprises a peptide.

16. The nucleic acid of claim 15, wherein the peptide comprises an artificial or in silico designed peptide.

17. The nucleic acid of claim 13, wherein the protein of interest encoded by the nucleotide sequence comprises one or more deletions, insertions, or substitutions as compared to its wild-type sequence.

18. A vector comprising any one of the nucleic acids of claims 1-17.

19. A host cell comprising a vector of claim 18.

20. A population of nucleic acids, each nucleic acid of the population comprising a mRNA display cassette, the mRNA display cassette comprising a nucleotide sequence encoding a protein of interest operably linked to (i) a nucleotide sequence encoding a MS2 bacteriophage coat protein (MCP) and (ii) to a nucleotide sequence encoding an RNA stem-loop, wherein the MCP binds to the RNA stem-loop with high-affinity.

21. A population of nucleic acids, each nucleic acid of the population comprising (i) a first cassette comprising a cloning site for insertion of a nucleotide sequence encoding a protein of interest operably linked to a nucleotide sequence encoding a MS2 bacteriophage coat protein (MCP) and (ii) a second cassette comprising a nucleotide sequence encoding a unique molecular identifier (UMI) sequence operably linked to a nucleotide sequence encoding an RNA stem-loop, wherein the MCP binds to the RNA stem-loop with high-affinity.

22. The population of nucleic acids of claim 20 or 21, wherein for each nucleic acid of the population the nucleotide sequence encoding the MCP is located 5′ to the nucleotide sequence encoding the protein of interest.

23. The population of nucleic acids of claim 20 or 22, wherein for each nucleic acid of the population the nucleotide sequence encoding the RNA stem-loop is located 3′ to the nucleotide sequence encoding the protein of interest.

24. The population of nucleic acids of claim 23, wherein for each nucleic acid of the population the nucleotide sequence encoding the RNA stem-loop is located in a 3′ UTR.

25. The population of nucleic acids of claim 20, wherein for each nucleic acid of the population the nucleotide sequence encoding the protein of interest and the MCP are operably linked so that they encode a fusion protein of the protein of interest and the MCP.

26. The population of nucleic acids of claim 25, wherein for each nucleic acid of the population the fusion protein comprises the MCP fused to the N-terminus of the protein of interest.

27. The population of nucleic acids of any one of claim 20-26, wherein for each nucleic acid of the population the mRNA display cassette further comprises a nucleotide sequence encoding a purification tag operably linked to the nucleic acid sequence encoding the protein of interest.

28. The population of nucleic acids of claim 27, wherein for each nucleic acid of the population the nucleotide sequence encoding the protein of interest and the purification tag are operably linked so that they encode a fusion protein of the protein of interest and the purification tag.

29. The population of nucleic acids of claim 28, wherein for each nucleic acid of the population the fusion protein comprises the purification tag fused to the C-terminus of the protein of interest.

30. The population of nucleic acids of any one of claims 20-29, wherein each nucleic acid of the population further comprises a promoter operably linked to the mRNA display cassette.

31. The population of nucleic acids of claim 30, wherein the promoter is an inducible promoter.

32. The population of nucleic acids of any one of claims 20-31, wherein for each nucleic acid of the population the mRNA display cassette further comprises a nucleotide sequence encoding a protein of interest.

33. The population of nucleic acids of claim 32, wherein at least one of the nucleic acids of the population comprises a nucleotide sequence encoding a protein of interest, wherein the nucleotide sequence comprises one or more deletions, insertions, or substitutions as compared to its wild-type sequence.

34. The population of nucleic acids of claim 32, wherein at least one of the nucleic acids of the population comprises a nucleotide sequence encoding a protein of interest, wherein the protein of interest encoded by the nucleotide sequence comprises one or more deletions, insertions, or substitutions as compared to its wild-type sequence.

35. The population of nucleic acids of any one of claims 20-34, wherein each nucleic acid of the population comprises a nucleotide sequence encoding a different protein of interest.

36. The population of nucleic acids of claim 32, wherein the protein of interest comprises a peptide.

37. The population of nucleic acids of claim 36, wherein the peptide comprises an artificial or in silico designed peptide.

38. The population of nucleic acids of any one of claims 20-37, wherein the nucleic acids of the population comprise nucleotide sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest.

39. The population of nucleic acids of any one of claims 20-38, wherein the nucleic acids of the population comprises nucleotide sequences encoding different proteins of interest that are representative of a proteome of interest.

40. The population of nucleic acids of claim 39, wherein the proteome of interest is the proteome of Saccharomyces cerevisiae.

41. The population of nucleic acids of any one of claims 20-40, wherein each nucleic acid of the population of nucleic acids is in a vector.

42. A population of host cells, wherein each host cell comprises a vector from the population of vectors of claim 41.

43. A method of producing a population of cells comprising an in vivo mRNA display library, the method comprising:
a) providing a population of cells, wherein each cell comprises one or more nucleic acid sequences encoding:
a RNA-binding protein;
a protein of interest; and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;
b) allowing the expression of the said one or more nucleic acid sequences;
c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest.

44. A method of producing a population of cells comprising an in vivo mRNA display library, the method comprising:
a) providing a population of cells, wherein each cell comprises one or more nucleic acid sequences encoding:
a RNA-binding protein;
a protein of interest;
a unique molecular identifier (UMI); and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the unique molecular identifier comprises the cognate RNA sequence;
b) allowing the expression of the said one or more nucleic acid sequences;
c) incubating the cells, thereby producing a population of cells comprising an in vivo mRNA display library, wherein the expressed RNA-binding protein-protein of interest fusion protein binds to the cognate RNA sequence present on the mRNA sequence encoding the unique molecular identifier.

45. The method of claim 43, wherein the RNA-binding protein is an RNA-binding capsid protein and optionally, wherein the cognate RNA-binding sequence is an RNA stem-loop sequence.

46. The method of claim 45, wherein the RNA-binding capsid protein is MS2 bacteriophage coat protein (MCP).

47. The method of any one of claims 43-46, wherein the nucleic acid sequence encoding the RNA-binding protein is located 5′ to the nucleic acid sequence encoding the protein of interest.

48. The method of any one of claims 43, 45-46, wherein the nucleic acid sequence encoding the cognate RNA sequence is located 3′ to the nucleic acid sequence encoding the protein of interest.

49. The method of claim 48, wherein the nucleic acid sequence encoding the cognate RNA sequence is located in a 3′ UTR of the mRNA sequence encoding the protein of interest.

50. The method of any one of claims 43-49, wherein the fusion protein comprises the RNA-binding protein fused to the N-terminus of the protein of interest.

51. The method of any one of claims 43-50, wherein the one or more nucleic acid sequences further encodes a purification tag wherein the nucleic acid sequence encoding the protein of interest and the purification tag are operably linked so that they encode a fusion protein of the protein of interest and the purification tag.

52. The method of claim 51, wherein the fusion protein comprises the purification tag fused to the C-terminus of the protein of interest.

53. The method of any one of claims 43-52, wherein each cell in the population of cells comprises a nucleic acid sequence encoding the same protein of interest.

54. The method of any one of claims 43-52, wherein each cell in the population of cells comprises a nucleic acid sequence encoding a different protein of interest.

55. The method of any one of claims 43-52, wherein the population of cells comprise nucleic acid sequences encoding at least 2, at least 10, at least 50, at least 100, at least 500, at least 1000, at least 2000, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, or at least 20,000 different proteins of interest.

56. The method of any one of claims 43-52, wherein the population of cells comprise nucleic acids sequences encoding different proteins of interest that are representative of a proteome of interest.

57. The method of any one of claims 43-52, wherein the nucleic acid sequence encoding the protein of interest comprises one or more deletions, insertions, or mutations as compared to its wild-type sequence.

58. The method of any one of claims 43-52, wherein the protein of interest encoded by the nucleic acid sequence comprises one or more deletions, insertions, or mutations as compared to its wild-type sequence.

59. A method of performing high throughput proteomics, the method comprising:
a) providing a population of cells, wherein each cell comprises one or more nucleic acid sequences encoding:
a RNA-binding protein;
a protein of interest; and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display, wherein the expressed RNA-binding protein-protein of interest fusion protein binds to the cognate RNA sequence present on the mRNA sequence encoding the protein of interest;
d) lysing the population of cells;
e) performing a biochemical assay on a portion of the lysate of step d) to generate an enriched lysate;
f) detecting the amount of mRNA encoding each protein of interest and comprising the RNA stem-loop that is present in the lysate of step d);
g) detecting the amount of mRNA encoding each protein of interest and comprising the RNA stem-loop that is present in the enriched lysate of step e); and
h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).

60. A method of performing high throughput proteomics, the method comprising:
a) providing a population of cells, wherein each cell comprises one or more nucleic acid sequences encoding:
a RNA-binding protein;
a protein of interest;
a unique molecular identifier (UMI); and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the UMI comprises the cognate RNA sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display, wherein the expressed RNA-binding protein-protein of interest fusion protein binds to the cognate RNA sequence present on the mRNA sequence encoding the UMI;
d) lysing the population of cells;
e) performing a biochemical assay on a portion of the lysate of step d) to generate an enriched lysate;
f) detecting the amount of mRNA encoding each UMI and comprising the RNA stem-loop that is present in the lysate of step d);
g) detecting the amount of mRNA encoding each UMI and comprising the RNA stem-loop that is present in the enriched lysate of step e); and
h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).

61. The method of claim 59 or 60, wherein the RNA-binding domain is an RNA-binding capsid protein and optionally, wherein the cognate RNA-binding sequence is an RNA stem-loop sequence.

62. The method of claim 59 or 60, wherein the detecting of steps f) and g) is performed using next generation sequencing.

63. The method of claim 59 or 60, wherein the detecting of steps f) and g) is performed by i) reverse transcribing the mRNAs encoding the proteins of interest and comprising the RNA stem-loop; ii) performing a second strand synthesis on the reverse transcription product; iii) fragmenting the second strand synthesis product; iv) ligating nucleic acid linkers to the fragmented nucleic acids; v) amplifying the ligated nucleic acids; and vi) sequencing the amplified nucleic acids.

64. The method of any one of claims 59-63, wherein the biochemical assay in an immunoprecipitation assay or a subcellular fractionation.

65. The method of any one of claims 59-64, wherein the plurality of different proteins of interest are representative of a proteome of interest.

66. The method of claim 65, wherein the proteome of interest is the proteome of the cells.

67. The method of any one of claims 59-66, wherein the cells are Saccharomyces cerevisiae.

68. The method of any one of claims 59-67, wherein the determining further comprises normalizing the amount of mRNA detected to the amount of mRNA detected of non-specific functional controls.

69. The method of claim 68, wherein the non-specific functional controls are proteins of interest represented in the plurality of proteins of interest but are not isolated by the biochemical assay.

70. A method of determining protein-protein interactions, the method comprising:
a) providing a population of cells, wherein each cell comprises one or more nucleic acid sequences encoding:
a RNA-binding protein;
a protein of interest; and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest;
d) lysing the population of cells;
e) incubating the lysate of step d)
f) performing proximity ligation to generate a plurality of hybrid sequences comprising the mRNA sequence encoding a first protein of interest and a mRNA sequence encoding one or more additional proteins of interest;
g) for each protein of interest, sequencing the hybrid sequence generated in step f);
h) for each protein of interest, identifying the one or more additional proteins of interest encoded by each hybrid sequence; wherein the additional proteins of interest of the plurality of hybrid sequences are identified as forming a protein-protein interaction with the first protein of interest.

71. A method of determining protein-protein interactions, the method comprising:
a) providing a population of cells, wherein each cell comprises one or more nucleic acid sequences encoding:
a RNA-binding protein;
a protein of interest; and
a nucleotide sequence encoding a unique molecular identifier (UMI) sequence operably linked to a nucleotide sequence encoding an RNA stem-loop, wherein the nucleic acid sequence encoding the RNA-binding protein and protein of interest are operably linked so that they encode a fusion protein of the RNA-binding protein and protein of interest; and wherein the nucleic acid sequence encoding the UMI and the RNA stem-loop are operably linked so that a mRNA encoding the protein of interest comprises the UMI sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display library, wherein the expressed RNA-binding protein-protein of interest fusion protein binds to the RNA stem-loop present on the RNA sequence encoding the UMI sequence and the RNA stem-loop sequence;
d) lysing the population of cells;
e) incubating the lysate of step d)
f) performing proximity ligation to generate a plurality of hybrid sequences comprising the RNA sequence encoding a first UMI and a RNA sequence encoding one or more additional UMIs;
g) for each hybrid sequence generated in step f), sequencing the one or more UMIs in the hybrid sequence;
h) determining the protein of interest associated with each UMI of each hybrid sequence in g); wherein the proteins of interest associated with a hybrid sequence are identified as forming a protein-protein interaction.

72. A method of determining protein-DNA or protein-RNA interactions, the method comprising:
a) providing a population of cells, wherein each cell comprises one or more nucleic acid sequences encoding:
a RNA-binding protein;
a protein of interest; and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display library, wherein the expressed RNA-binding protein-protein of interest fusion protein binds to the cognate RNA sequence present on the mRNA sequence encoding the protein of interest;
d) lysing the population of cells;
e) adding a population of DNA or RNA molecules to the lysate of step d);
f) incubating the mixture of lysate and a population of DNA or RNA molecules of step e)
g) performing proximity ligation to generate a plurality of hybrid sequences comprising the mRNA sequence encoding a protein of interest or a cDNA copy thereof and one or more of the DNA or RNA molecules;
h) for each protein of interest, sequencing the hybrid sequence generated in step g);
i) for each protein of interest, identifying the one or more DNA or RNA molecules of each hybrid sequence; wherein the one or more DNA or RNA molecules of the plurality of hybrid sequences are identified as forming an interaction with the protein of interest.

73. A method of determining protein-DNA or protein-RNA interactions, the method comprising:
a) providing a population of cells, wherein each cell comprises one or more nucleic acid sequences encoding:
a RNA-binding protein;
a protein of interest; and
a nucleotide sequence encoding a unique molecular identifier (UMI) sequence operably linked to a nucleotide sequence encoding an RNA stem-loop;
wherein the nucleic acid sequence encoding the RNA-binding protein and protein of interest are operably linked so that they encode a fusion protein of the RNA-binding protein and protein of interest; and wherein the nucleotide sequence encoding the UMI sequence and the RNA stem-loop are operably linked so that a mRNA encoding the RNA stem-loop comprises the UMI sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display library, wherein the expressed RNA-binding protein-protein of interest fusion protein binds with high affinity to the RNA stem-loop on the nucleotide sequence encoding the UMI sequence and the RNA stem-loop;
d) lysing the population of cells;
e) adding a population of DNA or RNA molecules to the lysate of step d);
f) incubating the mixture of lysate and a population of DNA or RNA molecules of step e)
g) performing proximity ligation to generate a plurality of hybrid sequences comprising the mRNA sequence encoding the UMI or a cDNA copy thereof and one or more of the DNA or RNA molecules;
h) for each hybrid sequence generated in step g), sequencing the UMI and the one or more DNA or RNA sequences of the hybrid sequence;
i) determining the protein of interest associated with each UMI of each hybrid sequence in h); wherein the one or more DNA or RNA molecules of the plurality of hybrid sequences are identified as forming an interaction with the protein of interest.

74. A method of determining protein-DNA or protein-RNA interactions, the method comprising:
a) providing a population of cells, wherein each cell comprises one or more nucleic acid sequences encoding:
a RNA-binding protein;
a protein of interest; and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the protein of interest and the cognate RNA sequence are operably linked so that a mRNA encoding the protein of interest comprises the cognate RNA sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display, wherein the expressed RNA-binding protein-protein of interest fusion protein binds to the cognate RNA sequence present on the mRNA sequence encoding the protein of interest;
d) lysing the population of cells;
e) performing a biochemical assay on a portion of the lysate of step d) to generate an enriched lysate; wherein the biochemical assay comprises enriching a DNA or RNA molecule of interest
f) detecting the amount of mRNA encoding each protein of interest and comprising the RNA stem-loop that is present in the lysate of step d);
g) detecting the amount of mRNA encoding each protein of interest and comprising the RNA stem-loop that is present in the enriched lysate of step e); and
h) for each protein of interest, comparing the amount of mRNA detected in step d) and step g), wherein a protein of interest is determined to be enriched under the biochemical assay conditions if an increased level of mRNA is detected in step g) compared to step d) or wherein a protein of interest is determined to be depleted under the biochemical assay conditions is a decreased level of mRNA is detected in step g) compared to step d).

75. A method of determining protein-DNA or protein-RNA interactions, the method comprising:
a) providing a population of cells, wherein each cell comprises one or more nucleic acid sequences encoding:
a RNA-binding protein;
a protein of interest;
a unique molecular identifier (UMI); and
a cognate RNA sequence, wherein the nucleic acid sequence encoding the RNA-binding protein and protein of interest are operably linked so that they encode a fusion protein of the RNA-binding protein and protein of interest; and wherein the nucleic acid sequence encoding the UMI and the cognate RNA sequence are operably linked so that a mRNA encoding the UMI comprises the cognate RNA sequence;
wherein the population of cells comprise nucleic acid sequences encoding a plurality of different proteins of interest;
b) expressing the said one or more nucleic acid sequences;
c) incubating the cells, thereby producing a population of cells comprising an in vivo mRNA display, wherein the expressed RNA-binding protein-protein of interest fusion protein binds to the cognate RNA sequence present on the mRNA sequence encoding the UMI;
d) lysing the population of cells;
e) performing a biochemical assay on a portion of the lysate of step d) to generate an enriched lysate; wherein the biochemical assay comprises enriching a DNA or RNA molecule of interest;
f) detecting the amount of mRNA encoding each UMI and comprising the RNA stem-loop that is present in the lysate of step d);
g) detecting the amount of mRNA encoding each UMI and comprising the RNA stem-loop that is present in the enriched lysate of step e); and
h) for each protein of interest, comparing the amount of mRNA detected in step f) and step g), wherein a protein of interest is determined to be enriched under the biochemical assay conditions if an increased level of mRNA is detected in step g) compared to step f) or wherein a protein of interest is determined to be depleted under the biochemical assay conditions is a decreased level of mRNA is detected in step g) compared to step f).
